(19) United States
(12) Patent Application Publication
(10) Pub. No.: US 2020/0321076 A1
(43) Pub. Date: Oct. 8, 2020

(54) STRUCTURAL VARIANT ANALYSIS

(71) Applicant: DOVETAIL GENOMICS, LLC, Scotts Valley, CA (US)

(72) Inventors: Nicholas H. PUTNAM, Waltham, MA (US); Christopher John TROLL, Santa Cruz, CA (US)

(21) Appl. No.: 16/762,619

(22) PCT Filed: Nov. 8, 2018
(86) PCT No.: PCT/US2018/059885

§ 371 Date: May 8, 2020

Related U.S. Application Data
(60) Provisional application No. 62/583,974, filed on Nov. 9, 2017.

(51) Int. Cl. G16B 20/20; C12Q 1/6869; G16B 30/20; G16B 40/00

(52) U.S. Cl. CPC G16B 20/20; G16B 40/00; G16B 30/20; C12Q 1/6869

(57) ABSTRACT

The disclosure provides methods, systems, and algorithms to identify and report genome or chromosome level structural information, such as the presence of structural variations. In some cases, structural variations include copy number variations, inversions, deletions, tandem duplications, or inverted duplications. Further provided herein are methods, systems and algorithms for assembling read-paired genomic data, including creating and optimizing scaffold models.

### CROSS-REFERENCE

This application claims the benefit of U.S. Provisional Application No. 62/583,974, filed Nov. 9, 2017, which is hereby explicitly incorporated by reference in its entirety.

### BACKGROUND

It remains difficult in theory and in practice to produce high-quality, highly contiguous genome sequences. This problem is compounded when one attempts to recover genome sequences, phasing information, or other genetic information is desired from preserved samples such as formalin-fixed, paraffin-embedded (FFPE) samples. Although a reduction in sequencing cost and time has increased the amount of raw genomic data available, a lack of suitable methods to analyze and assemble the data in an efficient and accurate way is a major limitation of current sequencing technology.

### INCORPORATION BY REFERENCE

All publications, patents, and patent applications mentioned in this specification are herein incorporated by reference to the same extent as if each individual publication, patent, or patent application was specifically and individually indicated to be incorporated by reference. All publications, patents, and patent applications mentioned in this specification are herein incorporated by reference in its entirety as well as any references cited therein.

### SUMMARY

Provided herein are methods of nucleic acid structural variant detection. Some such methods comprise a) mapping read pair information onto a reference nucleic acid scaffold; b) assigning a read pair position to a first bin such that the read pair midpoint falls within a first bin nucleic acid position range and the read pair separation falls within a first bin separation range; and c) estimating copy number variation based on a mappability value of the first bin. In some cases, the method further comprises normalizing the copy number variation. Additionally, the method further comprises visualizing mappability by plotting the mapped read density of two samples against each other.

Provided herein are methods of nucleic acid structural variant detection. Some such methods comprise a) mapping read pair information onto a reference nucleic acid scaffold; b) assigning a read pair position to a first bin such that the read pair midpoint falls within a first bin nucleic acid position range and the read pair separation falls within a first bin separation range; c) generating a two-dimensional image of the read pair information; wherein each pixel represents a bin; d) calculating a z-score for at least one group of four pixels sharing a common corner in the image; wherein the z-score is represented by a contrast between adjacent pixels; and e) identifying candidate hits when a z-score exceeds a threshold value. In some cases, the reference nucleic acid scaffold is a genome. Often, each data set is obtained from a different paired-end read direction. It is contemplated that the candidate hit is selected from one or more of a translocation, an inversion, a deletion, a duplication, and an interchromosomal structural variation.

Provided herein are systems for modeling a mixture of allelic variations in a sample. Some such systems comprise: a set of weighted genome scaffold models, wherein each genome scaffold model comprises a set of weighted chromosomes, wherein each chromosome is a linear graph of bins in the genome scaffold; and a module for calculating a log likelihood ratio of at least two genome scaffold models to predict whether a read pair sampled by a library will fall into a bin. In some cases, systems herein further comprise at least one feature detector module, wherein the at least one feature detector module proposes candidate modifications to the genome scaffold model. Often, the at least one feature detector module determines the bin boundaries of a sequence variant. It is contemplated that the sequence variant is selected from one or more of a translocation, an inversion, a deletion, and a duplication. Often the system further comprises a module that generates alternative models based on input from the at least one feature detector module.

Provided herein are methods for modeling allelic variations in a sample. Some such methods comprise: a) generating a set of weighted genome scaffold models, wherein each genome scaffold model comprises a set of weighted chromosomes, wherein each chromosome is a linear graph of bins in the genome scaffold; b) calculating a score based on the ability of the models to describe read pair sequencing information mapped on a reference sequence, wherein a higher score value indicates a more predictive model; and c) iteratively adding additional models to maximize the score value. It is contemplated that the read pair sequencing information comprises one or more of an inversion, a translocation, a duplication, and a deletion. In some cases, the methods further comprise detecting features, wherein detecting features comprises joining or separating bins in the model to increase the score value. Often, the sample is a cancer cell.

Provided herein are methods of nucleic acid structural variant detection. Some such methods comprise a) mapping read pair information onto a predicted nucleic acid scaffold; b) assigning a read pair position to a first bin such that the read pair midpoint falls within a first bin nucleic acid position range and the read pair separation falls within a first bin separation range; c) generating a two-dimensional image of the read pair information; wherein each pixel represents a bin; and d) identifying at least one feature in the two-dimensional image corresponding to two sequence fragments connected by a common linking sequence fragment. Often, the method comprises assembling the two sequence fragments connected by a common linking sequence fragment in the correct order. Sometimes, the method comprises discarding features corresponding to false positives.

Provided herein are methods comprising: mapping read pair sequence information onto a sequence scaffold; and identifying a local variation in density of a plurality of read pair symbols so mapped. In some cases, the method comprises assigning the local variation in density to a corresponding structural arrangement feature. Often, the method comprises restructuring the sequence scaffold so that the local variation in density is reduced. Sometimes, mapping read pair sequence information onto a sequence scaffold comprises positioning a symbol indicative of a read pair such that distance of the symbol from an axis representative of the sequence scaffold indicates distance from a mapped position of a first read of a read pair on the sequence scaffold to a mapped position of a second read of the read pair on the sequence scaffold, and such that position of the symbol relative to the axis representative of the sequence scaffold indicates an average of the mapped position of the first read of the read pair and the mapped position of the second read of the read pair. Sometimes, restructuring the sequence scaffold comprises reordering at least some contigs of the sequence scaffold. Alternatively or in combination, restructuring the sequence scaffold comprises reorienting at least one contig of the sequence scaffold. Often, restructuring the sequence scaffold comprises introducing a break into at least one contig of the sequence scaffold. Sometimes, the method further comprises introducing a sequence present at one edge of the break onto a second edge of the break. In some cases, restructuring the sequence scaffold comprises translocating a segment of a first contig into an internal region of a second contig. Sometimes, mapping read pair sequence information onto a sequence scaffold comprises assigning read pair information to a plurality of bins. Often, identifying a local variation in density comprises identifying a region having a locally low density of symbols. Alternatively, identifying a local variation in density comprises identifying a region having a locally high density of symbols. Sometimes, identifying a local variation in density comprises identifying a density at a first position and a density at a second position, wherein the density at the first position and the density at the second position differ significantly. In some cases, the first position and the second position are adjacent. Often, the first position and the second position are equidistant from the sequence scaffold. Sometimes, identifying a local variation in density comprises obtaining an expected density at a first position and an observed density at the first position. Often, the expected density at the first position is a density predicted by density gradient that decreases monotonically with increased distance from the axis representative of the sequence scaffold. Optionally, a local density variation of a fraction of a whole number value equal to a ploidy of a sample indicates an event in that proportion of a sample ploidy complement. In some cases, the scaffold represents a cancer cell genome. Alternatively or in combination, the scaffold represents a transgenic cell genome. Optionally, the scaffold represents a gene-edited genome. Often, the scaffold has an N50 of at least 20% greater following the restructuring.

Provided herein are methods comprising obtaining a scaffold comprising sequence scaffold information. Some such methods comprise obtaining paired read information; deploying the paired read information such that at least some read pair information is depicted so as to indicate position of each read in a read pair relative to the scaffold and to indicate distance of one read to another as mapped on the scaffold; and identifying a local variation in density of the paired read information as deployed. In some cases, the method comprises assigning the local variation in density to a corresponding structural arrangement feature. Sometimes, the method comprises reconfiguring the scaffold so as to decrease the local variation. Often, obtaining a scaffold comprising sequence scaffold information comprises sequencing a nucleic acid sample. Alternatively or in combination, obtaining a scaffold comprising sequence scaffold information comprises receiving digital information representative of a nucleic acid sample. Sometimes, the method comprises obtaining a predicted density distribution for deployed read pair information. Often, the identifying comprises identifying a significant difference between the predicted density distribution and the depicted read pair information density. Alternatively or in combination, identifying a local variation comprises identifying a density perturbation having a density peak at an apex of a right angle. In some cases, the apex of the right angle points to an axis representative of the scaffold. Often, obtaining paired end read information comprises crosslinking unextracted nucleic acids. Sometimes, obtaining paired end read information comprises crosslinking nucleic acids bound in chromatin. Often, the chromatin is native chromatin. Alternatively or in combination, obtaining paired end read information comprises binding a nucleic acid to a nucleic acid binding moiety. In some cases, obtaining paired end read information comprises generating reconstituted chromatin. Often, deploying the paired read information comprises assigning read pair information to a plurality of bins. Sometimes, restructuring the sequence scaffold comprises reordering at least some contigs of the sequence scaffold. Alternatively or in combination, restructuring the sequence scaffold comprises reorienting at least one contig of the sequence scaffold. Sometimes, restructuring the sequence scaffold comprises introducing a break into at least one contig of the sequence scaffold. Often, the method comprises introducing a sequence at one edge of the break onto a second edge of the break. Sometimes, restructuring the sequence scaffold comprises translocating a segment of a first contig into an internal region of a second contig. In some cases, the scaffold represents a cancer cell genome. Sometimes, the scaffold represents a transgenic cell genome. Alternatively or in combination, the scaffold represents a gene-edited genome. Often, the scaffold has an N50 of at least 20% greater following the restructuring. Sometimes, a local density variation of a fraction of a whole number value equal to a ploidy of a sample indicates an event in that proportion of a sample ploidy complement.

Provided herein are methods of identifying a structural rearrangement in a sample relative to a sequence scaffold. Some such methods comprise mapping read pair sequence information onto a sequence scaffold; identifying local density variation having a right angle edge pointing to an axis corresponding to the sequence scaffold and having bilateral symmetry along a line that bisects the right angle edge; and categorizing the sample as having a simple translocation relative to the sequence scaffold comprising segments of lengths from a translocation point at least as long as the longest furthest mapped read of the local density variation.

Provided herein are methods of identifying a structural rearrangement in a sample. Some such methods comprise mapping read pair sequence information onto a sequence scaffold; identifying local density variation having a right angle edge pointing to an axis corresponding to the sequence scaffold; identifying a sub-region of the local density variation that disrupts bilateral symmetry along a line that bisects the right angle edge; and categorizing the sample as having a translocation relative to the sequence scaffold comprising a segment that lacks sequence to which a population of symmetry-restoring read pairs would map.

Provided herein are methods of identifying a structural rearrangement in a sample relative to a sequence scaffold. Some such methods comprise mapping read pair sequence information onto a sequence scaffold; identifying local density variation having a right angle edge pointing to an axis corresponding to the sequence scaffold; obtaining an expected read pair density distribution curve; and identifying scaffold segments to which read pairs comprising the local density variation map; repositioning the scaffold segments such that the read pairs comprising the local density variation map to a region indicated by the expected read pair density distribution curve to have a density of the local density variation.

Provided herein are computer monitors configured to display results of any of the methods described herein.

Provided herein are computer systems configured to perform computational steps of any of the methods described herein.

Provided herein are visual representation of mapped read pair data described herein or generated using methods described herein.

Provided herein are methods of nucleic acid structural variant detection. Some such methods comprise mapping read pair information onto a predicted nucleic acid scaffold; obtaining a structural variant hypothesis; calculating a likelihood parameter that the structural variant hypothesis is consistent with the read pair information; and categorizing the nucleic acid sample as having the structural variant hypothesis if the likelihood parameter for the hypothesis is greater than a second likelihood parameter for a second hypothesis, wherein mapping read pair information onto a predicted nucleic acid scaffold comprises assigning a read pair a read pair position such that the read pair is assigned to its midpoint on the predicted nucleic acid scaffold on one axis; and such that the read pair is assigned a value corresponding to its read pair separation on a second axis Sometimes, said read pair comprises a first segment mapping to a first region of a nucleic acid molecule and a second segment mapping to a second region of the nucleic acid molecule, said first segment and said second segment being nonadjacent and sharing a common phase. Often, a read pair position is assigned to a first bin if the read pair midpoint falls within a first bin nucleic acid position range and the read pair separation falls within a first bin separation range. In some cases, the first bin nucleic acid position range is a regular interval of the predicted nucleic acid scaffold. Alternatively or in combination, the first bin separation range is a logarithmic interval of a full separation range for the read pair information. Sometimes, the first bin nucleic acid range is a regular interval of a nucleic acid scaffold, and wherein first bin separation range is a logarithmic interval of a full separation range for the read pair information. In some cases, a read pair position is assigned to a second bin if the read pair midpoint falls within a second bin nucleic acid position range and the read pair separation falls within a second bin separation range. Often, substantially all read information is binned. Sometimes, calculating the likelihood parameter comprises determining a likelihood contribution for the first bin. Often, the likelihood contribution for the first bin comprises a first likelihood factor proportional to a count of the read pairs mapping to the first bin. Alternatively or in combination, the likelihood contribution for the first bin comprises a second likelihood factor proportional to the area of the first bin. Sometimes, the likelihood contribution for the first bin comprises a first likelihood factor proportional to a count of the read pairs mapping to the first bin, and wherein the likelihood contribution for the first bin comprises a second likelihood factor proportional to the area of the first bin. Often, the method comprises determining a likelihood contribution for a second bin that does not overlap in area with the first bin. Sometimes, the likelihood parameter comprises the likelihood contribution of the first bin and the likelihood contribution of the second bin. Occasionally, the likelihood parameter comprises the likelihood contribution of a third bin. Alternatively or in combination, the likelihood parameter comprises a likelihood contribution for substantially all binned read pair information. Sometimes, the hypothesis comprises a structural variation having a left edge and a length. Often, the structural variation has an orientation that is at least one of a deletion, an inversion, a direct duplication, an outward inverted duplication, and an inward inverted duplication. Occasionally, the second hypothesis comprises a structural variant differing in at least one of a left edge, a length and a structural orientation. Sometimes, said nucleic acid structural variant is homozygous in said nucleic acid sample. Alternatively, said nucleic acid structural variant is heterozygous in said nucleic acid sample.

Provided herein are methods of visualizing a putative structural variation in a nucleic acid sample. Some such methods comprise the steps of assigning a population of sequence reads to a population of numbered bins, and assigning a likelihood parameter of a read comprising a structural variation edge falling within a first bin of said population of bins, wherein said likelihood parameter for said first bin comprises a first likelihood component that includes the number of reads mapping to the first bin and a second component that includes the area of the first bin. Sometimes, the method comprises plotting the likelihood of structural variation as a function of bin number. Frequently, said likelihood parameter for said first bin comprises a convolution of a first likelihood component that includes a number of reads mapping to the first bin and a second component that includes an area of the first bin. Alternatively or in combination, said likelihood parameter comprises a likelihood component relating a structural variant prediction to the number of reads mapping to the first bin and a likelihood component that includes the area of the first bin. Occasionally, said bin population shares a common bin width spanning a fixed nucleic acid distance. Sometimes, said bin population varies as to bin height among its members. Often, bin height appears constant when plotted on a logarithmic axis. Frequently, the likelihood parameter relates to a probability of a sequence read, comprising a junction of a structural variation having a left edge and a length, mapping to said first bin. Sometimes, the structural variation has an orientation that is at least one of a deletion, an inversion, a direct duplication, an outward inverted duplication, and an inward inverted duplication. Often, said sequence reads comprise read pairs. Occasionally, a read pair comprises a first segment mapping to a first region of a nucleic acid molecule and a second segment mapping to a second region of the nucleic acid molecule, said first segment and said second segment being nonadjacent and sharing a common phase.

Provided herein are methods of identifying a structural variant in a nucleic acid sample. Some such methods comprise the steps of obtaining mapped read pair data for the nucleic acid sample; obtaining a nucleic acid scaffold sequence; obtaining likelihood probability information for each of a plurality of structural variant hypotheses comparing the read pair data to the nucleic acid scaffold sequence; and identifying a most probable hypothesis among the structural variant hypotheses; wherein said method evaluates at least 10 Mb of nucleic acid scaffold sequence per minute. Frequently, the method comprises mapping read pair information onto the nucleic acid scaffold sequence; obtaining a structural variant hypothesis; calculating a likelihood parameter that the structural variant hypothesis is consistent with the read pair information; and categorizing the nucleic acid sample as having the structural variant hypothesis if the likelihood parameter for the hypothesis is greater than a second likelihood parameter for a second hypothesis. Occasionally, mapping read pair information onto the nucleic acid scaffold sequence comprises assigning a read pair a read pair position such that the read pair is assigned to its midpoint on the predicted nucleic acid scaffold on one axis; and the read pair is assigned a value corresponding to its read pair separation on a second axis Often, said read pair comprises a first segment mapping to a first region of a nucleic acid molecule and a second segment mapping to a second region of the nucleic acid molecule, said first segment and said second segment being nonadjacent and sharing a common phase. Sometimes, a read pair position is assigned to a first bin if the read pair midpoint falls within a first bin nucleic acid position range and the read pair separation falls within a first bin separation range. Occasionally, the first bin nucleic acid position range is a regular interval of a nucleic acid scaffold. Often, the first bin separation range is a logarithmic interval of a full separation range for the read pair information. Alternatively or in combination, the first bin nucleic acid position range is a regular interval of a nucleic acid scaffold, and wherein first bin separation range is a logarithmic interval of a full separation range for the read pair information. In some cases, a read pair position is assigned to a second bin if the read pair midpoint falls within a second bin nucleic acid position range and the read pair separation falls within a second bin separation range. Frequently, substantially all read information is binned. Often, calculating the likelihood parameter comprises determining a likelihood contribution for the first bin. Occasionally, the likelihood contribution for the first bin comprises a first likelihood factor proportional to a count of the read pairs mapping to the first bin. Sometimes, the likelihood contribution for the first bin comprises a second likelihood factor proportional to the area of the first bin. Alternatively or in combination, the likelihood contribution for the first bin comprises a first likelihood factor proportional to a count of the read pairs mapping to the first bin, and wherein the likelihood contribution for the first bin comprises a second likelihood factor proportional to the area of the first bin. Frequently, the method further comprises determining a likelihood contribution for a second bin that does not overlap in area with the first bin. Often, the likelihood parameter comprises the likelihood contribution of the first bin and the likelihood contribution of the second bin. Sometimes, the likelihood parameter comprises the likelihood contribution of a third bin. Occasionally, the likelihood parameter comprises a likelihood contribution for substantially all binned read pair information. Often, the hypothesis comprises a structural variation having a left edge and a length. Frequently, the structural variation has an orientation that is at least one of a deletion, an inversion, a direct duplication, an outward inverted duplication, and an inward inverted duplication. Sometimes, the second hypothesis comprises a structural variant differing in at least one of a left edge, a length and a structural orientation. Occasionally, said nucleic acid structural variant is homozygous in said nucleic acid sample. Alternatively, wherein said nucleic acid structural variant is heterozygous in said nucleic acid sample.

Provided herein are methods of selecting a treatment regimen. Some such methods comprise performing the method of any one of the preceding embodiments, identifying a rearrangement, and identifying a treatment regimen consistent with the rearrangement. Frequently, the treatment regimen comprises drug administration. Alternatively or in combination, the treatment regimen comprises tissue excision.

Provided herein are methods of evaluating a treatment regimen. Some such methods comprise performing the method of any one of the preceding embodiments a first time, administering the treatment regimen, and performing the treatment regimen a second time. Occasionally, the method comprises discontinuing the treatment regimen. Alternatively, the method comprises increasing dosage of the treatment regimen. Sometimes, the method comprises decreasing dosage of the treatment regimen. Alternatively, the method comprises continuing the treatment regimen. Frequently, the treatment regimen comprises a drug. Often, the treatment regimen comprises a surgical intervention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts an exemplary schematic of a protocol for analyzing read-pair library data.

FIG. 2A, FIG. 2B, and FIG. 2C depict a visual representation of read-pair library data for copy number variant estimation.

FIG. 2D depicts a visual representation of copy number variations between two samples.

FIG. 3A depicts a visual representation of mapped read pair data as a plot of read pair separation vs. the midpoint position of mapped read pairs for a sample matching a scaffold.

FIG. 3B depicts a visual representation of mapped read pair data as a plot of read pair separation vs. the midpoint position of mapped read pairs for a sample having an inversion.

FIG. 3C depicts an expanded scale visual representation of mapped read pair data as a plot of read pair separation vs. the midpoint position of mapped read pairs for a sample having an inversion.

FIG. 3D depicts an illustration of mapped read pair end data for a heterozygous inversion between points a and b.

FIG. 4A depicts an illustration of various types of structure variations, and the types of mapped read pair density patterns produced.

FIG. 4B depicts a generalized illustration of mapped read pair data observed for a structural variation.

FIG. 4C depicts a generalized illustration of mapped read pair data observed for a deletion.

FIG. 4D depicts a generalized illustration of mapped read pair data observed for an inversion.

FIG. 4E depicts a generalized illustration of mapped read pair data observed for an direct tandem duplication.

FIG. 4F depicts a generalized illustration of mapped read pair data observed for an inverted tandem duplication R.

FIG. 4G depicts a generalized illustration of mapped read pair data observed for an inverted tandem duplication L.

FIG. 5A depicts a visual representation of mapped read pair data as a plot of log likelihood ratio vs. bin number for a data set comprising an inversion.

FIG. 5B depicts a visual representation of mapped read pair data as a plot of log likelihood ratio vs. bin number for a data set with an area where the LLR is about 0.

FIG. 5C depicts a visual representation of mapped read pair data as a plot of log likelihood ratio vs. bin number for a data set with an area without a structural variation.

FIG. 6A and FIG. 6B depict exemplary simple kernels that can be used for finding reciprocal translocations.

FIG. 6C depicts a method for analyzing features using the ratio of foreground (fg) and background (bg) regions.

FIG. 6D depicts an image with identified features using a Z-score method.

FIG. 7 depicts an image of read pair data mapped on to a scaffold that illustrates an intra-chromosomal rearrangement.

FIG. 8A depicts an illustration of a “2^{nd} degree link” assembly situation, wherein two different assembly outcomes are possible from analyzing only first-order read pairs.

FIG. 8B, FIG. 8C, and FIG. 8D depicts an illustration of a “2^{nd} degree link” assembly situation using feature detection.

FIG. 8E depicts two plots showing the contribution of abundance of read pairs in a mixture (γ) and the gap size/distance (g) in predicting changes in mapped read pair density (contours).

FIG. 9 depicts an image with a feature corresponding to a reciprocal translocation between ETV6 and NTRK3.

FIG. 10A, FIG. 10B, and FIG. 10C depict image analysis-based results at the same pair of chromosomes compared in three different samples.

FIG. 11A, FIG. 11B, and FIG. 11C depict median normalized read density (over 10 samples) for chromosome 1 versus chromosome 7 (FIG. 11A), chromosome 2 versus chromosome 5 (FIG. 11B), and chromosome 1 versus chromosome 1 (FIG. 11C).

FIG. 12A and FIG. 12B depict various bin handling approaches. FIG. 12A shows equal bin sizes and FIG. 12B shows bin interpolation.

FIG. 13 depicts analysis by a genome-wide scanning analysis pipeline.

FIG. 14A and FIG. 14B depict read pair distance frequency data derived from FFPE-based ‘Chicago’ read pair libraries (FIG. 14A) and classic ‘Chicago’ based read pair libraries (FIG. 14B).

FIG. 15A and FIG. 15B illustrate the mapped locations on the GRCh38 reference sequence of read pairs that are plotted in the vicinity of structural differences between GM12878 and the reference. FIG. 15A depicts data for an 80 kb inversion with flanking 20 kb repetitive regions. FIG. 15B depicts data for a phased heterozygous deletion.

FIG. 16A depicts a displaced segment discrepancy in mapped read pair data as compared to a reference scaffold. In this case, a vertical segment of data (vertical line) has been displaced to an alternate “hole” section of the plot (arrow).

FIG. 16B depicts a collapsed segment discrepancy in mapped read pair data as compared to a reference scaffold. In this case, both segments B and B′ have been mapped on the scaffold to the same adjacent segment A.

FIG. 16C depicts collapsed repeat and misjoin discrepancy in mapped read pair data as compared to a reference scaffold. In this case, highly similar sequences B/X have been collapsed to a single assembly in the scaffold.

FIG. 17A depicts an exemplary workflow for iteratively improving a genome scaffold model to improve the quality of mapped read pair data on the scaffold.

FIG. 17B depicts an image of read pair data mapped on to a scaffold prior to model optimization for a potato chromosome.

FIG. 17C depicts an image of read pair data mapped on to a scaffold after model optimization for a potato chromosome.

FIG. 18A shows an exemplary computer system that is programmed or otherwise configured to implement the methods provided herein.

FIG. 18B illustrates an example of a computer system that can be used in connection with example embodiments of the present invention.

FIG. 18C is a block diagram illustrating a first example architecture of a computer system 700 that can be used in connection with example embodiments of the present invention.

FIG. 18D is a diagram demonstrating a network 2100 configured to incorporate a plurality of computer systems, a plurality of cell phones and personal data assistants, and Network Attached Storage (NAS) that can be used in connection with example embodiments of the present invention.

FIG. 18E is a block diagram of a multiprocessor computer system 900 using a shared virtual address memory space that can be used in connection with example embodiments of the present invention.

### DETAILED DESCRIPTION

Disclosed herein are methods and systems relating to detection, visualization and correction of rearrangements relative to a sequence scaffold as indicated by analysis of a nucleic acid sample. Rearrangements are in some cases indicative of molecular events occurring in some or all of the sample, such as genomic rearrangements that often occur in human or other cancer cells, as evaluated in comparison to a human reference genome. Alternate ‘rearrangements’ for which the present disclosure is relevant include draft or even previously published genome assemblies, for which substantial contig information may be available, but for which one or more contigs may be mis-positioned, such as by being placed out of order, mis-oriented relative to an experimentally determined sample, having collapsed regions of high similarity, or constructed using incorrectly joined contig constituents.

In both of these cases, practice of the methods and systems herein allows identification of discrepancies, if existent, between a scaffold of sequence information previously or concurrently generated, and data informative of short and long-range physical linkage information experimentally generated through the generation of pair reads. Discrepancies described herein are often referred to as kernels, features, or symbols.

Phasing information, chromosome conformation, sequence assembly, and genetic features including but not limited to structural variations (SVs), copy number variants (CNVs), loss of heterozygosity (LOH), single nucleotide variants (SNVs), single nucleotide polymorphisms (SNPs), chromosomal translocations, gene fusions, and insertions and deletions (INDELs) can be determined by analysis of sequence read data produced by methods disclosed herein. Other inputs for analysis of genetic features can include a reference genome (e.g., with annotations), genome masking information, and a list of candidate genes, gene pairs, and/or coordinates of interest. Configuration parameters and genome masking information can be customized, or default parameters and genome masking can be used.

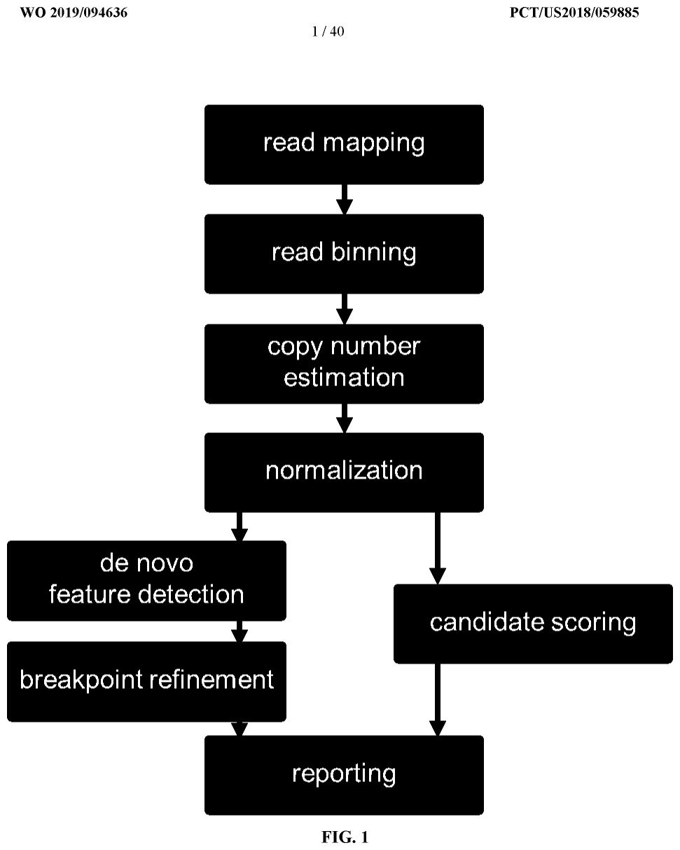

Methods described herein employ a variety of steps relating to processing of sequencing data. Optionally, each step utilizes a result or consideration from a previous step, and produces a result or output. In some cases steps are omitted or replaced with additional steps in a method workflow. In some instances, sequencing data (such as data generated pursuant to a Hi-C or other paired read protocol) is obtained by processing and sequencing of a sample. Exemplary steps for analysis of sequencing data often include read mapping (mapping paired sequence reads from one individual against a reference), read binning (group reads by one or more properties), copy number estimation (copy number variation, CNV), normalization, de novo feature detection, breakpoint refinement, candidate scoring, and reporting (FIG. 1). These steps are presented for example only, as other steps for identifying and reporting features are also used with the methods and systems described herein.

### Read Pair Generation

A number of read pair generation approaches are consistent with the disclosure herein. In exemplary embodiments, read pairs are generated using ‘Hi-C’ or related approaches using native or reconstituted chromatin to preserve linkage information among internally cleaved nucleic acid molecules such that a first region and a second region of a molecule are held together independent of their common phosphodiester backbone. However, the methods and systems herein are consistent with read pair data from a broad range of sources, and not all embodiments are limited by one or another read pair generation source.

### Mapping Read Pair Data

Common to many systems and methods herein is the generation of an array of binned read pairs that is optionally presented as a two-dimensional map relative to a scaffold sequence axis. Local density variations on such a map are identified, and contigs to which read pairs accounting for the local density variations are rearranged, reoriented, broken into fragments or otherwise manipulated so as to restructure the scaffold to which they contribute, so as to reduce overall or local density variation in a read pair binned array or a read pair distribution map.

As used herein, a read pair dataset is ‘mapped’ to a sequence scaffold when read pair data is binned or positioned relative to the scaffold sequence. In some cases the mapped data is depicted spatially, such as on a computer monitor or printed out. Alternately, a read pair dataset mapped to a sequence scaffold is stored as a data array on a data storage medium of a computer. Read pair data is preferably ‘binned’ or assigned to particular positions on a two-dimensional space or within a data array. Optionally, bins are represented by pixels in a computer generated image of the mapped read pair dataset.

Spatially depicted data is preferably presented such that read pair separation and the map location of individual reads of a read pair are captured in the positioning of a symbol representative of a read pair or occupied bin in a map.

For example some approaches to read pair data mapping comprise assigning a read pair to a bin that is positioned such that distance from the bin measured perpendicularly to an axis representative of scaffold sequence corresponds to or is indicative of the separation between where a first read and a second read of the read pair map or align most strongly onto the scaffold sequence. That is, read pairs having reads that align closely to one another on a scaffold are assigned to a bin close to the axis, while read pairs having reads that are separated from one another by a larger distance are assigned to bins that are further removed from the axis representing the sequence scaffold.

Optionally in combination, read pairs are positioned along an axis representing a scaffold sequence such that they are assigned a position or a bin that has a nearest point along the axis that represents the approximately or precisely the midpoint between the scaffold position to which the first read maps and the scaffold position to which the second read maps. Depending on the representation of the data, an axis can be referred to as a central axis, or diagonal (axis). In some cases the axis will be displayed horizontally, vertically, diagonally, or any other configuration.

In an example of a visualization, read pairs are mapped to a genome scaffold, and each pair is represented as a point in the plane with x and y coordinates equal to the distance between matching read pairs. The x-y plane can be divided into non-overlapping square bins and the number of read pairs mapping to each bin can be tabulated. The bin counts can be visualized as an image (e.g., a heat map) with bins made to correspond to pixels. In some cases, data from the read pair mapping described herein is visualized as a plot with a horizontal axis, or a 2D plot with intensity corresponding to read density. In some instances, data is processed and/or features are identified without a visualization step.

A low degree of ‘background’ is often observed in binning or read pair mapping. Such background manifests itself as single ‘night sky’ bin points in otherwise empty sectors of a data array or map visualization. Quantitatively, this background manifests itself as a very low local bin density in regions of a map or data array expected or otherwise indicated to be devoid of read pairs.

A number of technical factors separate from the disclosure herein account for such ‘night sky’ background. Factors include read pair sequence quality, sample or scaffold ‘GC percentage’ or base pair bias, overall or local repetitiveness in the genome, stringency or other technical parameters of read-to-scaffold alignment.

Errors in read sequence base calling may result in a read aligning to a scaffold region other than the region to which the underlying molecule is in fact derived from. Skewed GC percentages or repetitiveness lead to an increased chance that a read will align to multiple positions or that a single base error in sequencing may bring a read into alignment with an incorrect region of a scaffold. These chances may be reduced by adjusting base calling stringency in sequencing, or increasing stringency of assigning a read to a genomic region.

However, increases in stringency at either of these steps or elsewhere in the sequence generation and alignment processes also is likely to exclude from analysis a substantial amount of accurate, informative data. Thus, individual samples, sequencing protocols, organisms or experimental goals may dictate the degree to which ‘night sky’ background is tolerated in a given implementation of the methods or use of the systems as disclosed herein.

### Local Density Variation Determination

Pursuant to methods disclosed herein, it is often beneficial to assess local density variations in a read pair data array or mapped read pair dataset. A number of approaches are available for assessment of local density variation so as to identify a feature such as a kernel in a dataset array or mapped dataset.

Assessment of local density variation is made using any number of approaches known to one of skill in the art. For example, a local density is determined and compared to the density of an immediately adjacent region of a mapped read pair dataset or read pair array. Alternately, a local density is compared to the density of a region that is positioned a comparable or similar distance perpendicular to an axis defined by or corresponding to a scaffold sequence.

Rather than or in addition to a single comparison region, a local density variation is optionally detected by comparing a local density for an average density along a line or band that passes through the local region and runs parallel to the axis representative of the scaffold sequence. That is, the local density is compared to a density of read pairs sharing a common or comparable read pair separation but distributed at other positions throughout the scaffold.

Alternately or in combination, density values are determined for various positions throughout a map or a dataset, such that a density is compared to a local density of at least one other position of a map or dataset, such as 1, 2, 3, 4, 5, or more than 5 positions. A local density is determined and assessed relative to the a local density of at least one other position of a map or dataset, such that a local density variation can be matched to the position on a map or dataset having a common density, independent of the distance from the axis or average read pair distance of its members.

Similarly, in some cases a density gradient is determined, such as a density gradient that decreases as a function of distance from an axis, such as an axis representative of a sequence scaffold. A local density is then compared to densities of the gradient, and a local density is categorized as ‘variant’ if it differs significantly from the density gradient value at a distance from the axis comparable to the distance of the local density area to the axis. Differing ‘significantly’ can be evaluated by any number of statistical, computational, or other approaches known in the art or otherwise consistent with the disclosure herein.

Following such a determination, in some cases a ‘density predicted’ position for the read pairs responsible for the local density is determined, such that repositioning of scaffold constituents such as contigs on the axis results in the read pairs being positioned such that the local density matches or more closely approximates the local density of the read pairs following scaffold or scaffold contig repositioning.

Repositioning contigs or other scaffold constituents is effected so as to reduce a local density variation as assessed above, or to decrease a global measure of density variation relative to a global expected density gradient. Repositioning variously comprises reordering scaffold constituents such as contigs relative to one another, reorienting at least one contig relative to a second contig, breaking a contig into at least two constituents, introducing to a break point border a sequence such as sequence adjacent to the break, or excising a segment (or fragment) from a contig sequence and introducing the segment elsewhere in a contig of the scaffold.

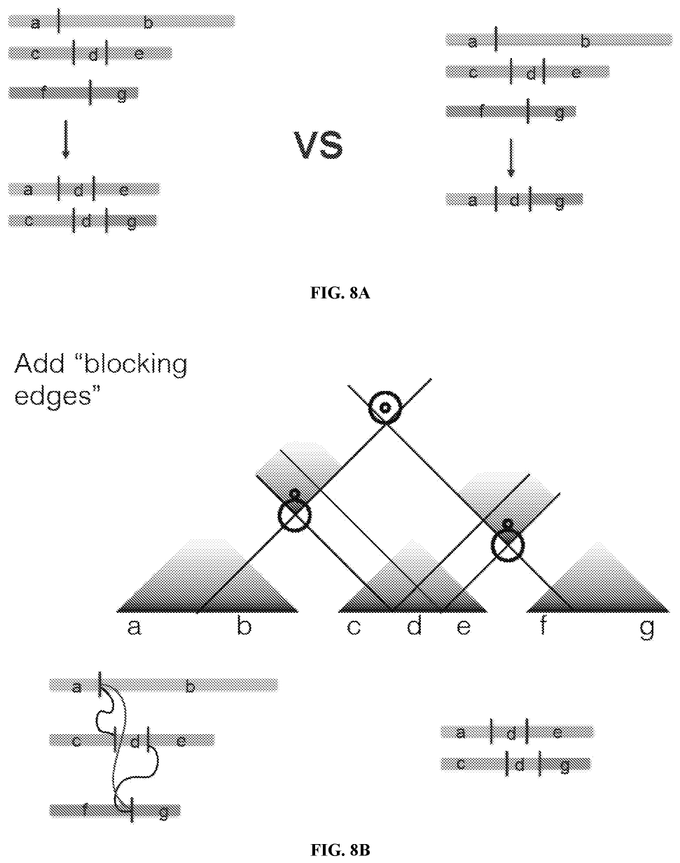

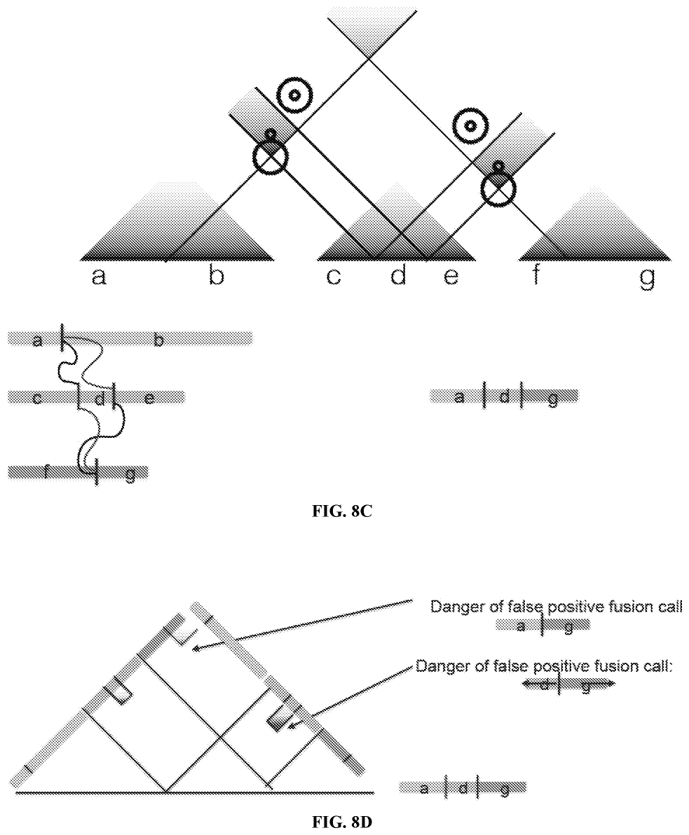

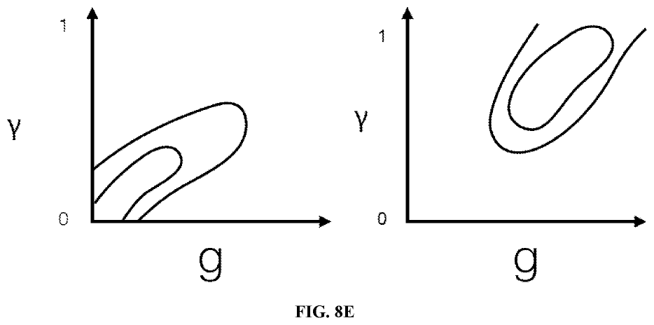

Expected density variation is in some aspects calculated using various modeling methods for predicting density. Optionally, a model relating y (mixture abundance) and g (gap-size) is used, wherein the contours indicate an expected rate of change (or gradient) in density. In this model, often the areas of steepest density change (contours) are found with low abundance/low gap size (FIG. 8E, left), and high abundance/high gap size (FIG. 8E, right). Additional models, including those based on empirically acquired data obtained from the methods and systems described herein are also predictive of changes in density, and are optionally incorporated throughout.

Local density in certain circumstances is defined as being “near” or “off” from defined areas on a mapped read pair plot. In some instances, an area defined as “near” a central axis corresponds to an area having an expected read density within at least 0.5×, 0.75×, 1×, 1.25×, 1.5×, 2×, or 2.5× of the mean expected density located exactly on the central axis. In some cases, an area defined as “off” a central axis corresponds to an area having an expected read density of no more than 0.1×, 0.2×, 0.3×, 0.4×, 0.5×, 0.75×, or no more than 0.9× of the mean density located on the central axis. Alternatively, areas defined as “near” the axis are described in terms of read pair separation distance (in base pairs) from the central axis. Optionally, a read pair distance of at least 1, 2, 5, 10, 20, 50, 100, 200, 500, 1000, 2000, 5000, 10,000, 20,000, 50,000, 100,000, 200,000, 500,000, 1 million, 2 million, 5 million, 10 million, or at least 20 million base pairs from the central axis is defined a “off” the axis. In some cases, a read pair distance of about 1, 2, 5, 10, 20, 50, 100, 200, 500, 1000, 2000, 5000, 10,000, 20,000, 50,000, 100,000, 200,000, 500,000, 1 million, 2 million, 5 million, 10 million or about 20 million base pairs from the central axis is defined a “off” the axis. Similarly, a read pair distance of no more than 1, 2, 5, 10, 20, 50, 100, 200, 500, 1000, 2000, 5000, 10,000, or no more than 20,000 base pairs from the central axis is defined a “near” the axis. Similarly, a read pair distance of about 1, 2, 5, 10, 20, 50, 100, 200, 500, 1000, 2000, 5000, 10,000, or about 20,000 base pairs from the central axis is defined as “near” the axis. Alternately, read pair distances are represented by bins, wherein each bin represents a range of read pair distances in base pairs.

In various manifestations of the methods described herein, the read density between two defined areas is compared to establish a boundary or presence of a kernel. In some cases this difference is at least 10%, 20%, 50%, 80%, 100%, 200%, 500%, 800%, 1000%, 2000%, 5000%, or at least 5000%. In other instances this difference is about 10%, 20%, 50%, 80%, 100%, 200%, 500%, 800%, 1000%, 2000%, 5000%, or at least 5000%.

In various manifestations of the methods described herein, the difference in read density between an observed density and an expected density is compared (“higher” or “lower”) to identify a discrepancy between a model scaffold and mapped read pair data. In some cases this difference is at least 10%, 20%, 50%, 80%, 100%, 200%, 500%, 800%, 1000%, 2000%, 5000%, or at least 5000%. In other instances this difference is about 10%, 20%, 50%, 80%, 100%, 200%, 500%, 800%, 1000%, 2000%, 5000%, or at least 5000%.

### Complex Rearrangement Assessment

Read pair bin array or map analysis in some cases indicates bin distributions consistent with particular rearrangements relative to a sequence scaffold. Often, a particular rearrangement has multiple impacts or signatures on a bin array or map, depending upon the extent or relatedness of multiple events in a rearrangement on a molecule such as a chromosome or in a predicted sequence such as a scaffold sequence.

Upon identifying a local density variation in a data array or map indicative of a rearrangement, through some methods and systems herein one is taught to survey for secondary local density variations or for details of a local density variation indicative of the extent or co-occurrence of multiple events in a rearrangement. For example, a simple translocation event results in a characteristic local density distribution that, if occurring involving fragments of lengths that are greater than a density resolution of a map or binned data array, will yield a symmetrical local density distribution. However, if the translocation or scaffold rearrangement is of an internal segment rather than a full arm of a molecule or scaffold, then, provided that the segment is within a density resolution of a map or binned data array, one may see one or more perturbations. The local density distribution indicative of the event may lack bilateral symmetry along a line bisecting the local density variation at its closest point to the axis. Alternately or in combination, a second local density distribution is detected involving read pairs having one read that maps to the region where one expects reads that would restore symmetry to the previous local density variation if mapped to the first local density variation. Such a density distribution is often indicative of a complex rearrangement in a sample molecule or scaffold such that two breakpoints join three distinct segments relative to the starting or expected scaffold.

An exemplary complex rearrangement “2^{nd} degree link” situation is illustrated in FIG. 8A. Sequences a-g (FIG. 8A, top) are divided at the sites shown to form fragments (labeled a-g), and rearranged to form products (FIG. 8A, bottom). The common linkage of both fragment a and g to fragment d complicates the analysis, which would produce signals consistent with both a-d-e/c-d-g and a-d-g reassembled fragments. However, both scenarios are in some cases distinguished by identifying an additional long-range signal a-g of a-d-g that is present in FIG. 8B, and absent from FIG. 8A (a-d-e/c-d-g). In some instances, further methods are used to reduce the possibility of false positive fusion calls that would result from observing these long-range signals (FIG. 8D). In one method of reducing false positives, all fusion calls are grouped by shared break-points, and fusion calls are rejected if they share both break points with a higher-scoring call. In another method of reducing false positives, a model-based discrimination method is applied to examine likelihood as a function of y (mixture abundance) and g (gap-size) (FIG. 8E), wherein the contours predict an expected rate of change in density.

### Local Density Variation Geometry

Local density variations often manifest themselves in a mapping output as having at least one right angle edge ‘pointing’ toward the axis, such that a line locally bisecting the angle represents the shortest distance from the local density variation to the axis.

Some local density variations are square, exhibiting bilateral symmetry along a line drawn perpendicular to the axis and bisecting a right angle edge pointing toward the axis.

Alternately, some local density variations exhibit bilateral symmetry as above but have a distal edge or border that is poorly defined, owing to the local density variation being substantially greater at the right angle edge pointing toward the axis relative to elsewhere in the local density variation.

Alternately, some local density variations are rectangular rather than square, lacking bilateral symmetry along a line drawn perpendicular to the axis and bisecting a right angle edge pointing toward the axis. In extreme cases such local density variants appear to be linear at lower levels of resolution. In addition, local density variations are observed having configurations other than those as described above.

Alternately, some local density variations are “bow tie” shaped, wherein a center point is defined approximately midway between a segment length and at the same distance away from the axis. Four regions of density intersecting at right angles at the center point are in some cases observed, with the boundary lines of the regions intersecting the axis at a 45 degree angle, and passing through the boundaries of the segment on the axis. One region of density is optionally bounded by the axis, and in some cases, the regions adjacent to the axis-bounded region have a higher than expected density.

Information from Local Density

Method and systems disclosed herein allow for local density determinations to be used toward a number of ends in various approaches herein.

Peak variation for a local density variation, such as is seen at a right angle edge closest to an axis representing scaffold sequence, is in some cases informative as a measure of copy number of the genomic event to which it relates. That is, a local density variation indicative of adjacent segments, alone or in combination with other map or bin array information, is assayed as to its peak density. This density is compared to peak density immediately off axis for the map or dataset. Metrics used variously comprise mean, median, mode or other measure of on-axis density.

Comparisons indicating a whole number ratio of one to the other indicate in some cases the ploidy of the event associated with the local density variation. That is, a density of one half the local axis density indicates an event that is haploid in diploid sample. A density of one eighth the local axis density indicates an event that is occurs on one chromosome of an octoploid sample. A density of five eighths the local axis density indicates an event that is occurs on five chromosomes of an octoploid sample. Other combinations are apparent to one of skill in the art, such as ¼, ½, or ¾ in a tetraploid genome, 1, 2, 3, 4, 5, 6, 7, or 8 of 8 in an octoploid genome, 1, 2, 3, 4, 5, or 6/6 in a hexaploid genome, or other proportions involving or approximating whole number rations within a range consistent with sample genome ploidy. Similarly, heterogeneity of a collection of genes will also in some instances give rise to whole number variations in local density. For example, a density appears at 1/10 the expected density for a haploid sample, indicating that 1/10 of the genomes comprise the event. These events are often manifested in heterogeneous cell populations, such as a tumor or other population of diverse cells.

Alternately or in combination, peak density for a local density variation, such as is seen at a right angle edge closest to an axis representing scaffold sequence, is in some cases informative as a measure of distance between edges of the genomic event to which it relates relative to the scaffold sequence. That is, a local density variation indicative of physically linked segments, alone or in combination with other map or bin array information, is assayed as to its peak density. This density is compared to a density gradient ranging from immediately off axis for the map or dataset, decreasing to a background density further off the axis. Metrics used variously comprise mean, median, mode or other measure of on-axis density to determine points on the density gradient.

Density of a local density variation is determined and compared to a read pair bin density gradient so as to find an off-axis distance on the gradient having a comparable density. The scaffold sequence is then reconfigured so as to position the reap pairs of the local density variation such that their density matches that of the gradient. Accordingly, scaffold constituents are reconfigured so as to reduce overall density variation in the data array or in the map relative to a gradient.

For an idealized set of read pair data mapped onto a perfect scaffold, almost all of the density is equally distributed on a central axis. Alternatively, the distribution of density is predicted using a model of the data, such that an expected density or density gradient decreasing from the axis is generated. Areas of high or low density relative to the expected density on the diagonal axis are in some instances indicative of discrepancies between read pair data and the scaffold model. For example, an area having a higher than expected density on the axis in some cases indicates a collapsed fragment in the scaffold model. In another example, an area having a lower than expected density on the axis in some instances indicates a misjoin between two fragments in the scaffold model. In one aspect, a misjoin incorrectly connects two chromosomes together. On-axis density variations in some aspects describe any number of discrepancies between the observed read pair data and the scaffold model.

### Mathematical Models of Density

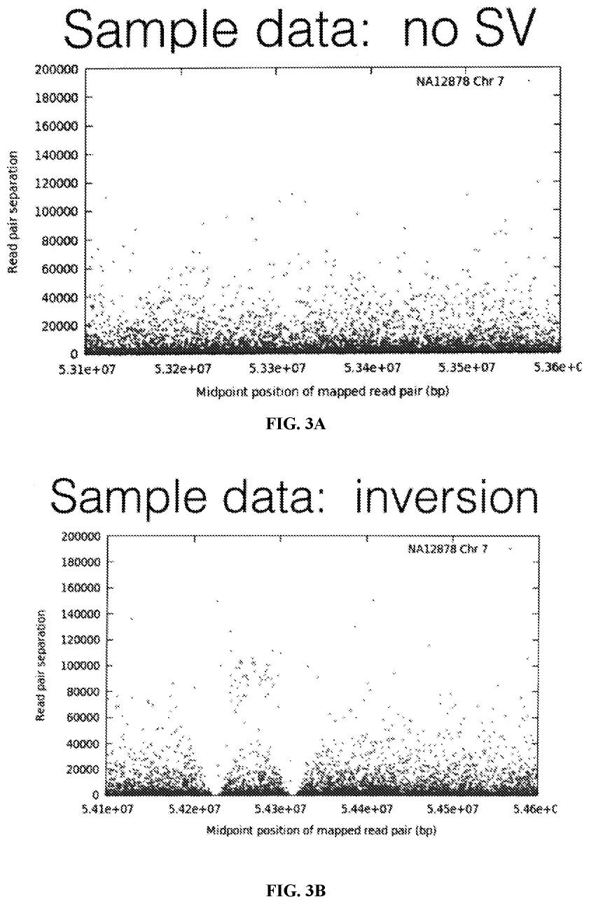

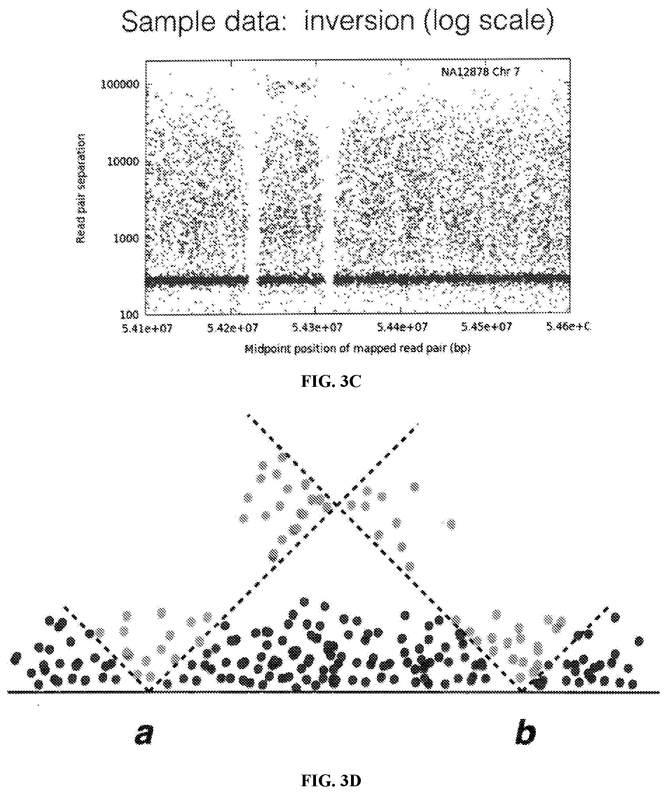

In one aspect of density data processing, a plot of genome location (for example, represented by the midpoint position of a mapped read pair) is plotted against read pair separation. In a genome without a structural variation (SV, discrepancies, features, etc.), the majority of points are distributed near the baseline (FIG. 3A). However, the presence of a variation, such as an inversion, produces a plot such as that depicted in FIG. 3B and FIG. 3C. The areas near the baseline that lack points represent the edges of the inverted segment. The structural variation is in some instances modeled as a feature or kernel, as shown in FIG. 3D, wherein sites a and b are the edges of the event, with the light colored points representing those that are now reflected above the midpoint of a and b (intersection of the dotted lines), which often is used to identify the feature. Optionally, a likelihood ratio is calculate comparing the hypotheses 1) an SV exists in the genome and 2) the genome matches the reference. In some cases, a hypothesis h is formulated as linear operations, including expressing the data in the region of interest as a set of read pair counts in bins: Cᵢⱼ and set Aᵢⱼ to the area of each bin, calculating the log likelihood ratio (LLR) contribution per read pair (S^{h}ᵢⱼ) for the i,j bin, and calculating the log likelihood contribution per unit area of the i,j bin (T^{h}ᵢⱼ). In one exemplary equation, a LLR score is expressed as:

S^{h}=ΣᵢⱼS^{h}ᵢⱼCᵢⱼ+ΣᵢⱼT^{h}ᵢⱼAᵢⱼ

In some instances, it is beneficial to calculate a likelihood ratio for a plurality of SVs. For example, a pair (S^{h}ᵢⱼ, T^{h}ᵢⱼ) is used to search for an SV at every offset k in the genome:

S^{h}ₖ=Σ_{i,j}S^{h}_{i,j}C_{i-k,j}+Σ_{i,j}T^{h}_{i,j}A_{i-k,j}

Wherein the process is optionally repeated to calculate the likelihood ratio for all SVs in the genome.

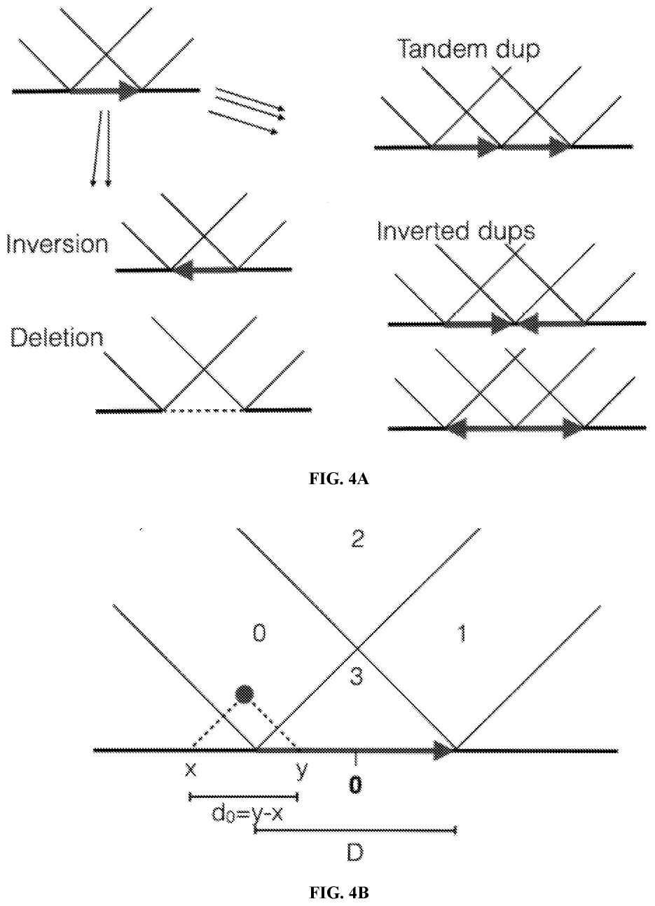

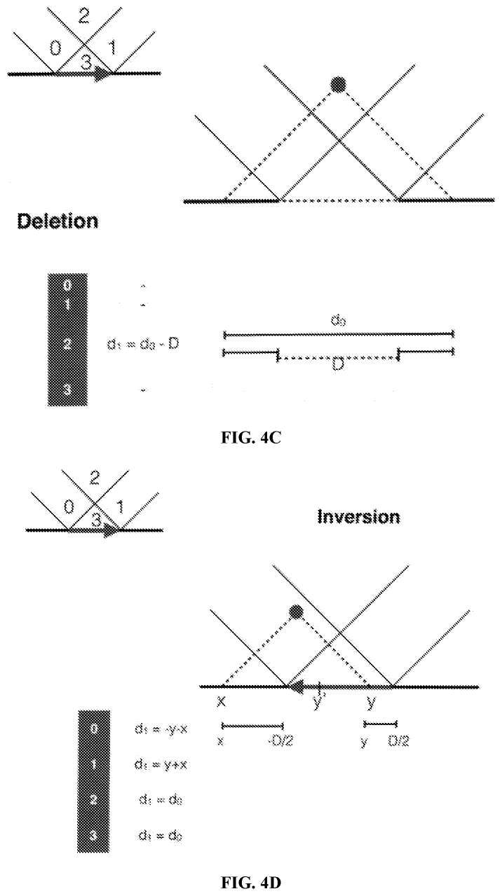

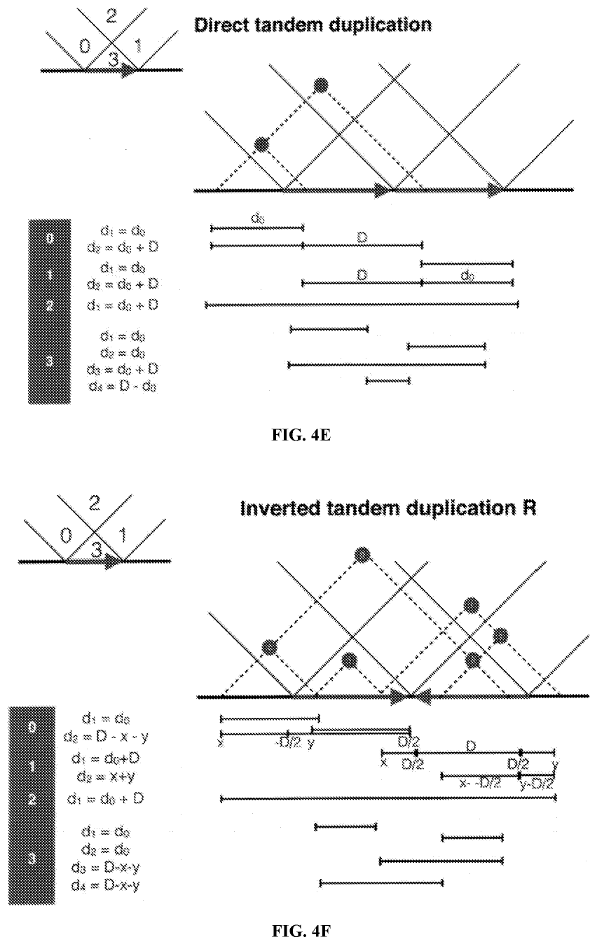

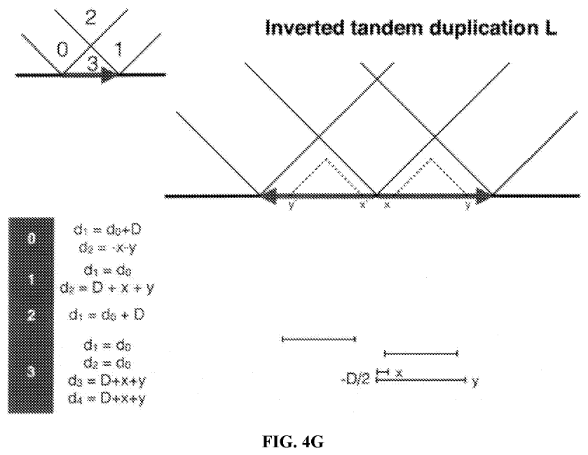

In another instance, each of the variations in FIG. 4A are analyzed. By way of example only, each variation including inversion, deletion, tandem duplication, and inverted duplication have read pairs mapping with an apparent separation d₀, and possible true separations dᵢ in the genome. In some cases dᵢ is determined for each of the four regions (0, 1, 2, 3) in the variations depicted in FIGS. 4B-4G.

Read pair separation changes often are changed into kernel elements using for example, the Chicago likelihood model represented by the equation:

$$L = {N}^{n} {e}^{- Np} {\prod }_{j = 1}^{n} {P}_{j}$$

where n represents hits to “rare” outcomes out of N tries, and p is the total probability of the rare outcomes:

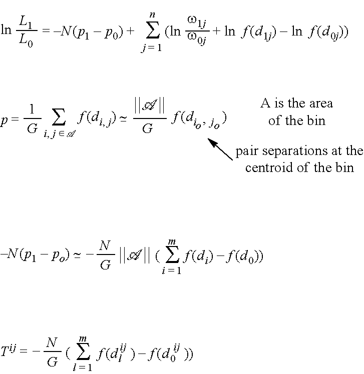

m is the multiplicity of the alternative scenarios, in the case of duplications.

$${S}^{ij} = ln ( {\sum }_{l = 1}^{m} f ( {d}_{l}^{ij} ) ) - ln ( f ( {d}_{0}^{ij} ) )$$

or optionally for the heterozygous case:

$${T}^{ij} = - \frac{N}{G} ( \frac{1}{2} f ( {d}_{0}^{ij} ) + \frac{1}{2} {\sum }_{l = 1}^{m} f ( {d}_{l}^{ij} ) - f ( {d}_{0}^{ij} ) ) = - \frac{N}{2 G} ( {\sum }_{l = 1}^{m} f ( {d}_{l}^{ij} ) - f ( {d}_{0}^{ij} ) )$$

$${S}^{ij} = ln ( \frac{1}{2} f ( {d}_{0}^{ij} ) + \frac{1}{2} {\sum }_{l = 1}^{m} f ( {d}_{l}^{ij} ) ) - ln ( f ( {d}_{0}^{ij} ) ) - ln ( f ( {d}_{0}^{ij} ) )$$

Occasionally, a bin will overlap a region boundary for a feature or kernel. One potential solution comprises calculating areas and centroids for each overlap region, using max( ) for S^{h}_{i,j} and min( ) for T^{h}_{i,j}. As appreciated by one skilled in the art, alternative feature analysis equations and algorithms are also used with the methods and systems herein.

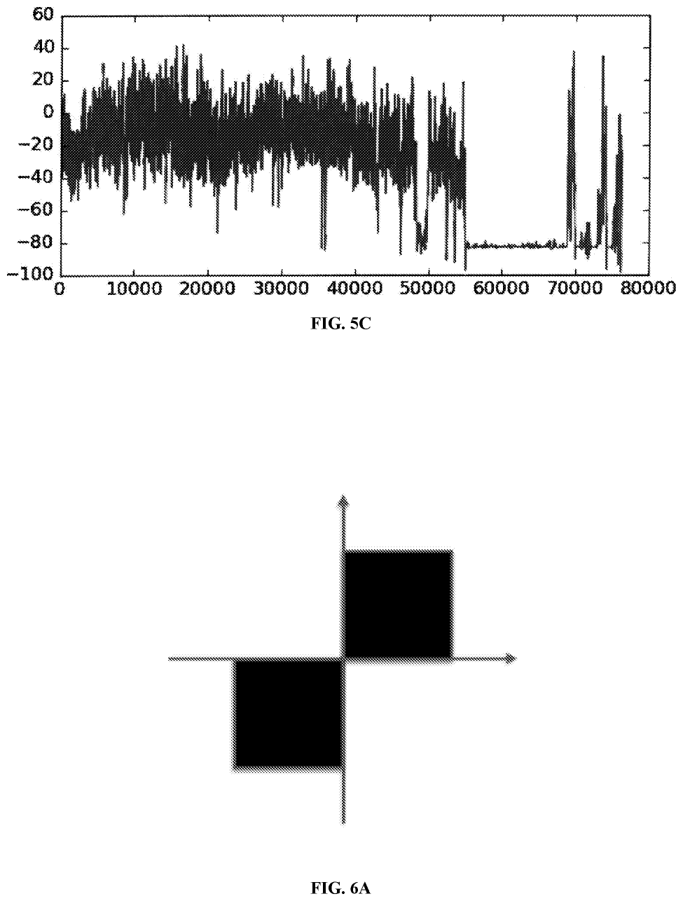

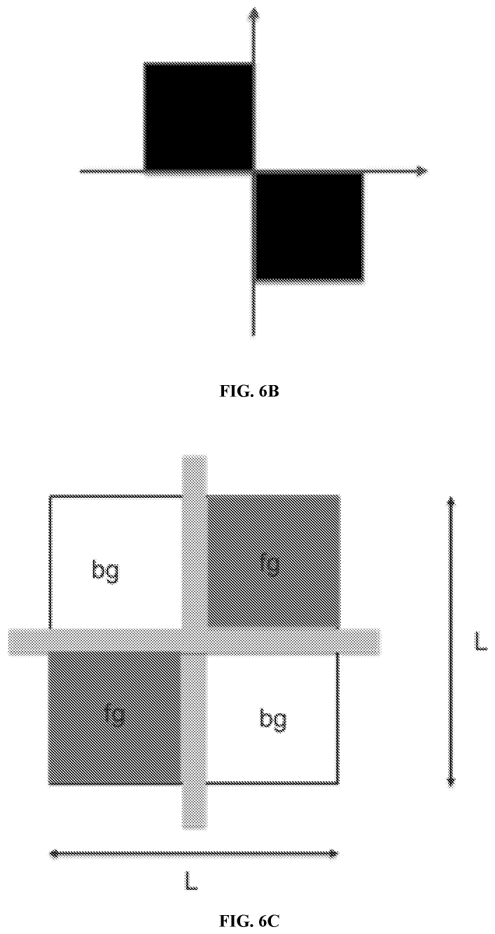

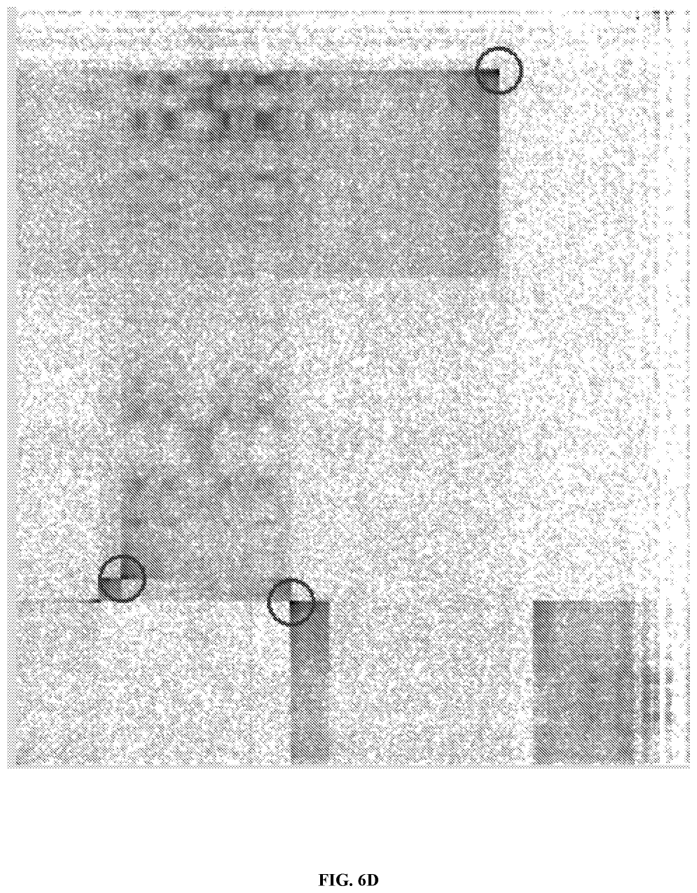

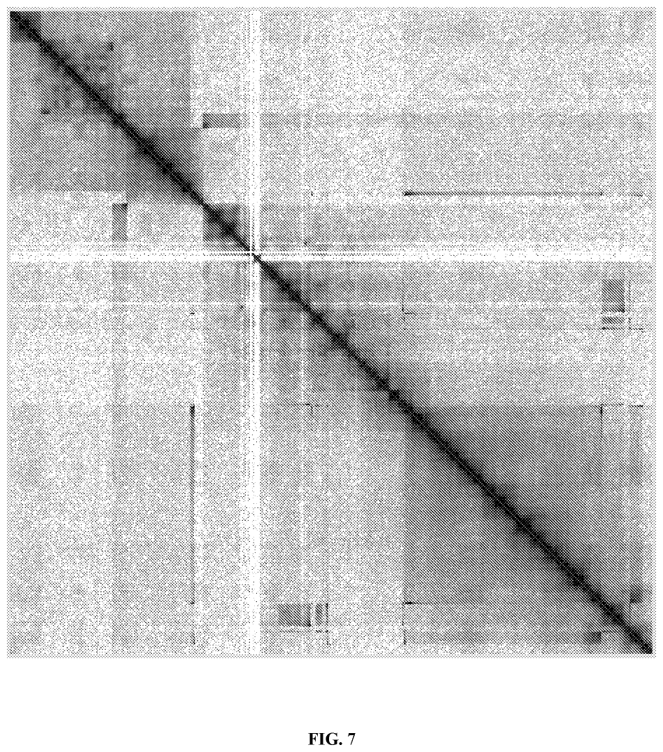

Additional analysis techniques, such as image processing techniques, are variously used to identify the signatures of genetic features such as different rearrangements. For example, kernel convolution filtering can be used to find points in the image corresponding to pairs of genomic loci that are fused, by analyzing a two dimensional plot of paired reads. FIG. 6A and FIG. 6B show exemplary simple kernels that can be used for finding reciprocal translocations. In various cases a local z-score is calculated for a kernel by computing a z-score contrast value defined as the ratio of foreground to background areas of the kernel, which is repeated for each pixel (FIG. 6C). An exemplary image with features identified from z-scoring (circled) is shown FIG. 6D. In some instances, a reciprocal translocation between ETV6 and NTRK3 is identified (FIG. 7). The “bowtie” shaped feature in the upper right and lower left quadrants is indicative of interaction between these two regions of the genome characteristic of a reciprocal translocation. In some aspects, interchromosomal rearrangements are identified with the method of local z-score detection. This process is optionally repeated for every pixel in the image. In some cases, all local maxima that exceed a threshold are considered candidate hits for a feature.

### Scaffold Modeling

The relationship between nucleic acid fragments (contigs, clusters, etc.) is in some instances represented by a mathematical graph model, wherein each sequence is a node, and the interface between any two fragments in an assembly is represented as an edge connecting two or more nodes. A path connecting all nodes through edges (and only crossing each node once) represents in some cases a solution to the assembly of sequencing fragments. Often, a lack of unique overlap regions in sequencing data fragments leads to a plurality of solutions (or paths) for assembly. For example, in an idealized haploid series of fragments A, B, and C, one envisions 6 different options (or paths) for connecting all three fragments in a linear fashion. However, if edges between nodes A/B, and B/C are manifested as a kernel on a graph of mapped read pair density on or near the central axis with a scaffold model corresponding to the arrangement A-B-C, then the model accurately matches a single path, A-B-C. In certain cases, a region corresponding to an edge (for example edge A/B) is absent of density corresponding to a feature, the arrangement now contains a “blocking edge” that informs the scaffold model, and reduces the number of likely paths. A blocking edge in some cases prevents a path from being defined between two nodes of the graph model, informing the assembly that these two fragments are not adjacent. Optionally, each edge is given a weighting factor that dictates the likelihood of utilizing that edge as part of a solution path. The weighting factor in some cases represents a likelihood that the two nodes are connected. For a scaffold model of A-B-C, in some instances a lower than expected density will be observed on the diagonal where the feature for A-B is expected, which would decrease the weighting factor of edge A-B. In a practical sense, this in some instances allows for simplification of the number of paths through nodes for a graph model of the sequences. In another example, a feature corresponding to the edge A-C is observed at the intersection of a horizontal line bisecting the location of fragment A on the axis, and a vertical line bisecting the location of fragment C on the axis. For a scaffold model of A-B-C, this in some cases indicates node (or fragment) B has been incorrectly placed in the scaffold model between fragments A and C, which should be adjacent.

More complicated translocation events are often aided by addition of blocking edges. For example, FIG. 8A depicts two different rearrangements/paths (left and right), that each possess edges connecting fragments a/d and d/g. This assembly situation and various others are often treated by application of a graph theory model. By adding a blocking edge between a/g (top concentric circles, FIG. 8B) corresponding to a lack of mapped read density, only a single path connecting a-d-e and c-d-g is most likely. Alternatively, by adding a blocking edge between a/e and c/g (two sets of concentric circles, FIG. 8C) given the lack of density in the two regions represented by the concentric circles, only a single path corresponding to a-d-g is likely. Optionally, more complex translocation events are also analyzed using this general strategy.

### Evaluation of Models

Entire scaffolds, chromosomes, or genomes consisting of many fragments (nodes) can in some aspects be described using this method, for which many assembly solutions represented by paths through the nodes are evaluated. Often variants exist as intra-chromosomal variants, and are addressed using various methods of data analysis, such as modeling that are defined by a plurality of potential equations. In one exemplary method of data analysis, a genome model “scaffold” is built from a sequencing data set, such as a Hi-C data set. Optionally the data is acquired from a tumor, and comprises a mixture of genomes, or any other sample that heterozygous for an allele. In some aspects, a set of genomes comprising a high degree of genetic heterogeneity (such as a tumor) is modeled as a weighted set of genome models, defined by the equation:

[symbol]

={(α₁, [symbol] ₁), (α₂, [symbol] ₂), . . . }

wherein each genome (G₁, G₂, etc.) is defined as a weighted (weighting factor α) model of a set of chromosomes. In some cases, each chromosome (C) is defined as a linear graph of bins on the genome:

[symbol]

={ [symbol] ₁, [symbol] ₂, . . . }

In some embodiments, the number of read pairs mapping to connect a pair of genome bins (i,j) is defined as a Poisson distribution:

$$P ( n ) = \frac{{λ}^{n} {e}^{- λ}}{n !}$$

An exemplary equation for a log likelihood ratio of two models predicting λ₁ and λ₂ reads respectively is:

$$\begin{matrix}ln \frac{{L}_{1}}{{L}_{2}} = n ln \frac{{λ}_{1}}{{λ}_{2}} - ( {λ}_{1} - {λ}_{2} ) \\ = n ln \frac{{p}_{1}}{{p}_{2}} - ( {\overline{n}}_{1} - {\overline{n}}_{2} )\end{matrix}$$

In some aspects the model provides the probability that a read pair samples by the library from the genome will fall in bin i,j. For an isotropic model (without a trans-activation domain, (TAD)), the probability is optionally expressed as:

$${p}_{ij} = \sum {α}_{g} \frac{{ω}_{i} {ω}_{j}}{{\overline{G}}^{2}} p ( {d}_{ij}^{g} )$$

Where d^{g}_{i,j} is the shortest-path distance between bins i and j in the genome g, and p(d) is the empirical read path separation distribution. Alternately or in combination, the read pair probability is elaborated with copy number and mappability terms for bins i and j. In some cases, a non-isotropic model comprising a location-specific TAD is used:

$${p}_{ij} = \sum {α}_{g} \frac{{w}_{i} {w}_{j}}{{\overline{G}}^{2}} p ( {d}_{ij}^{g} ) {𝒯}_{ij}$$

or a more general form:

$${p}_{ij} = \sum {α}_{g} \frac{{w}_{i} {w}_{j}}{{G}^{2}} {p}_{ij}^{g}$$

Modifications and improvements to the model often increase the quality and accuracy of the data. Often a new component is added to the model to increase the model's ability to describe the data. For example, a sequence of models Mk is generated to improve the initial model which was generated from the reference scaffold, or a comparison genome scaffold. It often is assumed that [symbol] ₖ₊₁ adds one new genome [symbol] ₖ₊₁ to [symbol] ₖ with weight γ and the weights αi for 1<i<k are each updated to (1−γ)αi. Given multiple candidates for Mk+1, in some cases the candidate leading to the greatest increase in score ΔS is selected:

$$\begin{matrix}Δ S = ln \frac{{L}_{k + 1}}{{L}_{k}} = {\sum }_{ij} {S}_{ij} \\ = {\sum }_{ij} - γ ( {\overline{n}}_{ij}^{k + 1} - {\overline{n}}_{ij}^{k} ) + {n}_{ij} ln [ γ \frac{{p}_{ij}^{k + 1}}{{p}_{ij}^{k}} + 1 - γ ]\end{matrix}$$

For example, in some instances the best model is found by selecting a γ which maximizes ΔS. Alternately or in combination, all the weights αᵢ are adjusted to obtain an increased ΔS.

In some aspects, new mixture component candidates are acquired which lead to large values of ΔS when summed over all (i, j). However, often the contribution to ΔS of these potential model components are concentrated in the ij plane near fusion junctions. In some instances, local image filtering identifies candidate edits. When such a local search identifies a high-scoring (and therefore not explained by the current model) contact between bins r and s, this contact optionally is either added in a new “genome” or as an edit to one of the genomes already in the mixture. Feature detection methods in some cases propose candidate modifications to the model to explain the features that are found. For example, a basic set of feature detection methods comprises one or more of: “reciprocal translocation+”, “reciprocal translocation−”, “translocation++”, “translocation+−”, “translocation−+”, “translocation−−”, or “break” methods. The feature detector methods often output features, for example: break after bin i, break before bin j, or join bin i to bin j. In some instances, a method takes a list of features and the model, and generates alternative models for scoring. For example, if a model already consists of n alternative genomes, the method optionally applies the edits of the feature to each of these n, and makes a new copy of each to apply the edits to, for a total of 2n alternative models. Other scoring models are also utilized during the practice of this method.

In another feature identification technique, modeling is used to identify intra-chromosomal rearrangements. For example, the likelihood that a rearrangement has occurred often is determined by calculating a log likelihood ratio (LLR) is as the ratio between two hypotheses:

$$S = ln \frac{{L}_{1}}{{L}_{2}} = - ( {\overline{n}}_{1} - {\overline{n}}_{2} ) + {\sum }_{j = 1}^{n} ln \frac{{P}_{j}^{1}}{{P}_{j}^{2}}$$

Where n̅₁ is the expected number of reads in a region of the 2D contact plane under hypothesis i, and Pⁱⱼ is the probability of sampling a read pair with the separation implied by hypothesis i for read pair j, given the insert size distribution model. In some instances, the hypothesis are background and background plus signal mixed in a frequency λ. In some aspects the hypotheses are a) variation exists in the area of the genome under analysis, and b) the genome matches the reference. For example, to compute the LLR score S for two hypotheses: (1) the reads were generated from a mixture of genomes in which a fraction contains a fusion between loci i and j relative the reference, and (0) no such contact exists near i, j.

$$\begin{matrix}S = - ( γ {\overline{n}}_{1} + ( 1 - γ ) {\overline{n}}_{0} - {\overline{n}}_{0} ) + {\sum }_{j = 1}^{n} ln \frac{γ P ( {d}_{1} ) + ( 1 - γ ) P ( {d}_{0} )}{P ( {d}_{0} )} \\ = - γ ( {\overline{n}}_{1} - {\overline{n}}_{0} ) + {\sum }_{j = 1}^{n} ln [ γ \frac{P ( {d}_{1} )}{P ( {d}_{0} )} + 1 - γ ]\end{matrix}$$

The score contributed by n reads relating two small bins on the genome separated by a gap d₀, positioned relative to the contact being tested (i, j) such that the reads would be separated by d₁ in the rearranged genotype (a small region of the 2D contact plane) often is expressed as follows (making a small bin approximation):

$$dS = - γ ( {\overline{n}}_{1} - {\overline{n}}_{0} ) + n ln [ γ \frac{P ( {d}_{1} )}{P ( {d}_{0} )} + 1 - γ ]$$

The score S is the sum over the plane of contributions dS within w bins in each direction i, j.

$${S}_{ij} = {\sum }_{k = - w}^{w} {\sum }_{l = - w}^{w} - γ ( {\overline{n}}_{1}^{k , l} - {\overline{n}}_{0}^{i + k , j + l} ) + {n}^{i + k , j + l} ln [ γ \frac{P ( {d}^{k , l} )}{P ( {d}^{i + k , j + l} )} + 1 - γ ]$$

In some cases the score “S” with regard to y estimates variant abundance. In the limit where γ→1, this becomes separable, and amenable to calculation with kernel convolution:

$$\begin{matrix}{S}_{ij} = {\sum }_{k = - w}^{w} {\sum }_{l = - w}^{w} - {\overline{n}}_{1}^{k , l} + {\overline{n}}_{0}^{i + k , j + l} ln P ( {d}^{k , l} ) - {n}^{i + k , j + l} ln P ( {d}^{i + k , j + l} ) & ( 6 ) \\ = {N}_{1}^{K} + {N}_{0}^{ij} + {( {K}_{S 1} * M )}^{ij} - {( {K}_{0} * Q )}^{ij} & ( 7 )\end{matrix}$$

Wherein M is the matrix of observed reads counts, K_{S1} is a feature detection kernel with elements ln P(d^{k,l}), K₀ is a trivial kernel with elements equal to 1 and covering the footprint of the kernel, Q is the null hypothesis read likelihood contribution with elements equal to the elementwise product of M and P(d) (similar to diagonal distance contours), N^{K}₁ is a constant representing the number of reads expected from the rearranged genotype in range of the kernel, and N₀ is the matrix with elements indicating the number of reads expected under hypothesis 0 (diagonal contours). To first order in 1→γ,

$${S}_{ij} = {\sum }_{k = - w}^{w} {\sum }_{l = - w}^{w} - γ ( {\overline{n}}_{1}^{k , l} - {\overline{n}}_{0}^{i + k , j + l} ) + {n}^{i + k , j + l} [ ln \frac{P ( {d}^{k , l} )}{P ( {d}^{i + k , j + l} )} + ( 1 - γ ) ( \frac{P ( {d}^{i + k , j + l} )}{P ( {d}^{k , l} )} - 1 ) ]$$

In some cases it is reasonable to approximate this (e.g. gamma<1) as

Sᵢⱼ=−γ(N₁^{K}−N₀^{ij})+(K_{S1}*M)^{ij}−(K₀*Q)^{ij}

since the term

$$( 1 - γ ) ( \frac{P ( {d}^{i + k , j + l} )}{P ( {d}^{k , l} )} - 1 )$$

is often small where P(d^{k,l})>>P(d^{i+k,j+l}).

In some aspects, a likelihood function determines contig order and orientation. In some cases, the likelihood function is derived from the multinomial probability of observing a particular configuration of N balls cast into k+1 bins, numbered 0, 1, . . . , k, where xᵢ is the number of balls (or paired-end reads) falling into the i^{th} bin, and Pᵢ is the probability that a ball will land in bin i:

$$p ( x ) = \frac{N !}{{x}_{0} ! {x}_{1} ! {x}_{2} ! … {x}_{k} !} {\prod }_{i = 0}^{k} {P}_{i}^{{x}_{i}}$$

In one example, bin 0 has a much higher probability than the remaining “rare” bins. If n«N balls fall into m of the “rare” bins, and the remaining N−n balls end up in bin 0, then the probability often is described as

$$p ( x ) = \frac{N !}{( N - n ) !} \frac{1}{{x}_{1} ! {x}_{2} ! … {x}_{k} !} {P}_{0}^{N - n} {\prod }_{j = 1}^{m} {P}_{j}^{{x}_{j}}$$

where j indexes the rare bins that receive a ball. Without loss of generality, in some instances bins are renumbered 1 . . . k such that the first m of them are the ones that get hit by a ball. The remaining factors of Pᵢ^{xi} (for the bins where i>m and xᵢ=0) are all equal to 1. Optionally a further assumption that the rare bins are so rare that none are ever hit by more than one ball is applied, and m=n, reducing the equation to:

$$p ( x ) = \frac{N !}{( N - n ) !} {P}_{0}^{N - n} {\prod }_{j = 1}^{n} {P}_{j}$$

By the normalization condition on the Pi, and defining p for convenience as the combined probabilities of all the rare bins:

$${P}_{0} = 1 - {\sum }_{i = 1}^{k} {P}_{j} = 1 - p$$

From the Poisson limit theorem, if N is very large and p is very small:

$$\frac{N !}{( N - k ) ! k !} {{p}^{k} ( 1 - p )}^{N - k} \approx \frac{{λ}^{k}}{k !} {e}^{- λ}$$

where λ=Np. In some aspects, this simplifies the combinatorial factors in the expression for the probability. In some instances, the substitution n=k is made, and the approximation is re-written as:

$$\frac{N !}{( N - k ) ! k !} {{p}^{k} ( 1 - p )}^{N - k} \approx Poisson ( n ; Np ) \frac{n !}{{p}^{n}}$$

$$p ( x ) \approx Poisson ( n ; Np ) \frac{n !}{{p}^{n}} {\prod }_{j = 1}^{n} {P}_{j} = {( Np )}^{n} {e}^{- Np} {\prod }_{j = 1}^{n} \frac{{P}_{j}}{p}$$

The log probability in some cases is expressed in the following ways:

$$ln p ( x ) \approx n ln N + n ln p - Np + {\sum }_{j = 1}^{n} ln {\tilde{P}}_{j} = n ln [ Np ] - Np + {\sum }_{j = 1}^{n} ln {\tilde{P}}_{j} = n ln \overline{n} - \overline{n} + {\sum }_{j = 1}^{n} ln {\tilde{P}}_{j} = n ln \overline{n} - \overline{n} - n ln p + {\sum }_{j = 1}^{n} ln {P}_{j} = n ln N - \overline{n} + {\sum }_{j = 1}^{n} ln {P}_{j}$$

In some cases, Pᵢ is normalized to

$${\tilde{P}}_{j} = \frac{{P}_{j}}{p} .$$

Often the Poisson approximation to the Binomial distribution is used which governs n, which often is valid as long as N is large and Np=n̅<<N, and the assumption that at most one ball lands in a given rare bin. In some instances, the log likelihood ratio is expressed as:

$$S = ln \frac{{L}_{1}}{{L}_{2}} = - ( \overline{{n}_{1}} - \overline{{n}_{2}} ) + {\sum }_{j = 1}^{n} ln \frac{{P}_{j}^{1}}{{P}_{j}^{2}}$$

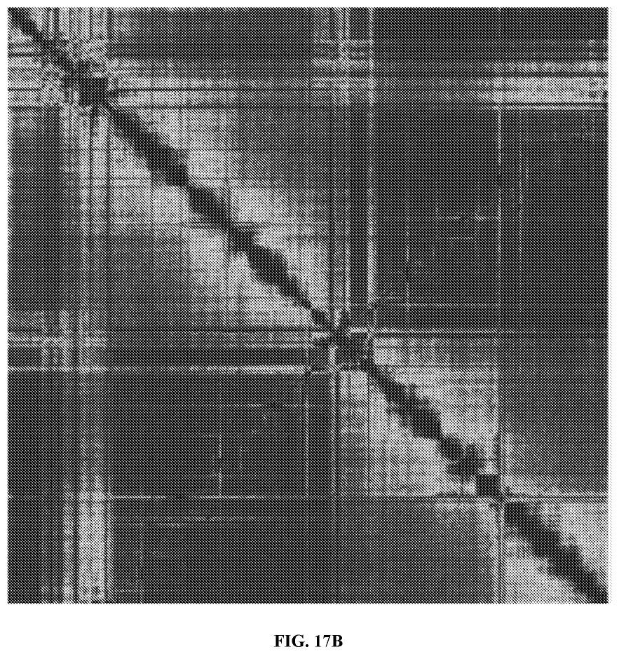

Optimization of the scaffold model in some cases results in lowering of the score S, indicating a model that better describes the data. This optimization process is optionally repeated until all discrepancies between the model and the mapped read pair data are removed. At FIG. 17A one sees an exemplary workflow for improving a scaffold model, including steps of obtaining raw link density data, generating a contact potential score, making side graph edits, generating a distance field, and updated the contact potential relative to the current side graph. In some cases, this process results in an interactively updated graph-based model of a genome. In some instances, this process is iterated to improve the quality of mapped read pair data for feature identification. Contact potential score in some instances are generated for every potential feature (or discrepancy) in the plot. Side graph edits in some cases refer to changing the weight given to edges in the graph model of the assembly, which influences the most likely assembly solution. In some aspects, these side graph edits correspond to reordering fragments in the scaffold, removing fragments, duplicating fragments, or breaking fragments to create better agreement between the scaffold model and the read pair data. Once edits are made, the shortest path through the graph model is often identified, and the read pair data is mapped onto the new scaffold model. In another step, all potential discrepancies between the scaffold model and the read pair data are reevaluated and a new score is generated. Optionally, these steps are repeated to minimize the overall score, indicating a more accurate scaffold assembly. The overall effect in some cases is observed visually, for example in the difference between FIG. 17B obtained before optimization of the model, and FIG. 17C obtained after.

Other equations and methods for genome modeling and expressing probability are also used with the methods and systems described herein.

### Copy Number Estimation

Calculation of copy number variation often is beneficial for evaluating disease states, for example in evaluating the number of gene copies that possess a mutation associated with a cancer. Copy number estimation for mutations is determined using a broad number of approaches, such as approaches related to density assessment of local density variations relative to other fields or positions of a map, or relative to a density gradient field. In some cases, copy number variation is calculated using the equation:

$${N}_{i}^{obs} \sim P ( {N}^{T} \frac{w}{G} {c}_{i} {m}_{i} )$$

$${c}_{i} = \frac{{N}_{i}}{N} \frac{G}{w} \frac{1}{{m}_{i}}$$

Wherein Nᵢ is the number of mapping reads in bin i, N is the total number of reads mapped, w is the bin width, G is the genome size, cᵢ is the copy number of bin i, and mᵢ is the mappability of bin i. The mappability in some aspects refers to the ability to reassemble a section of a genome, which in some cases is hampered by highly repetitive sequences. In some cases, the cᵢ is biased towards 1 if Nᵢ and mᵢ are both small. In some instances, a chromosome is divided into bins, and mapped read pairs are sorted into bins based on the midpoint of the pair. In some instances, the number of read pairs linking genome bins i and j follows the equation:

Nᵢⱼ˜P(cᵢcⱼmᵢmⱼ N pᵢⱼ)

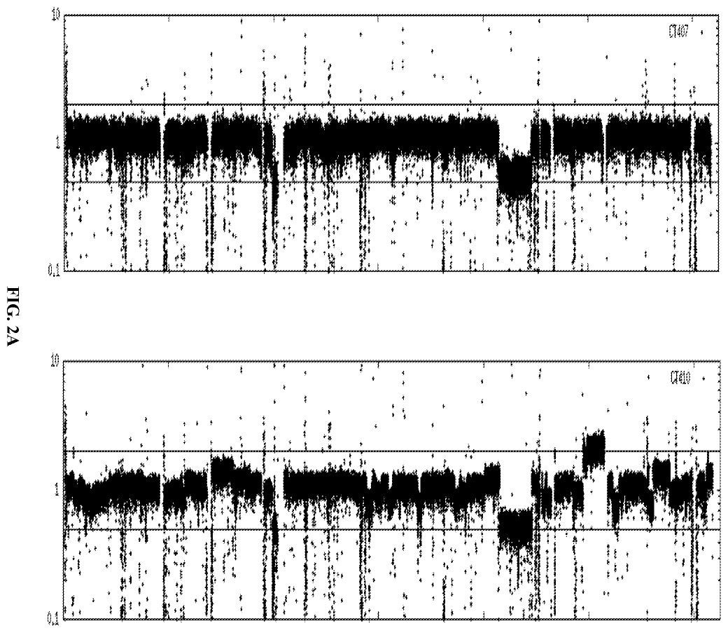

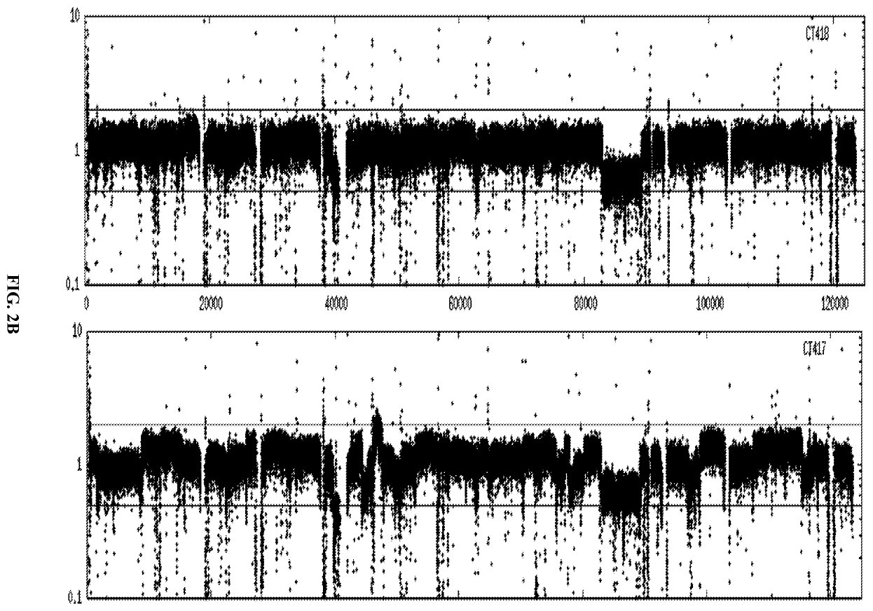

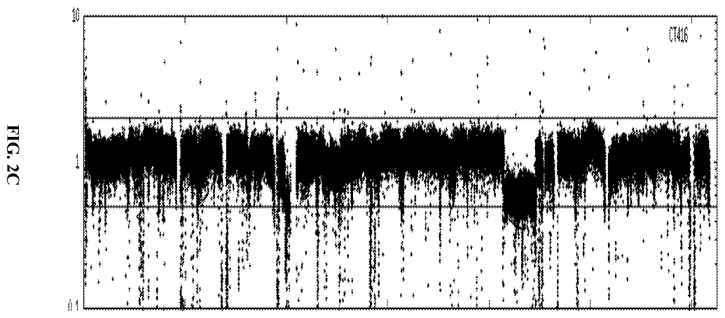

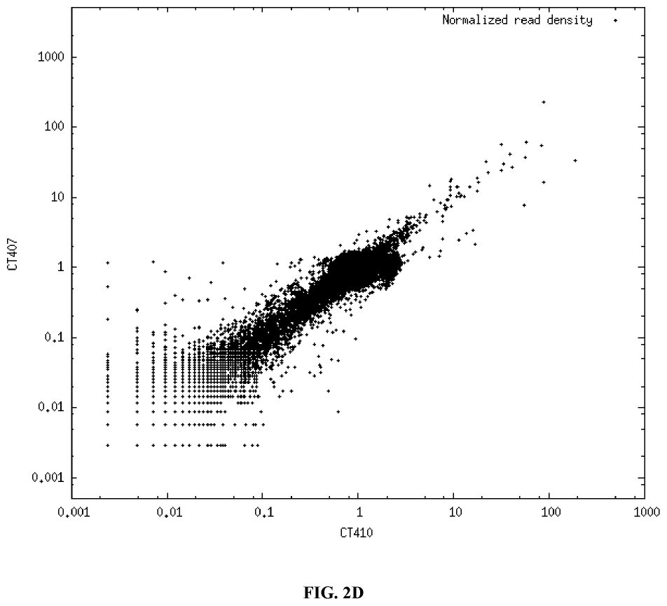

A 2D histogram is in some cases generated to visually display copy number data of different samples (FIGS. 2A-2C). In another aspect, the 2D histogram is normalized to isolate the signal of long-range contacts from copy number differences:

$${\overline{N}}_{ij} = \frac{{N}_{ij}}{{c}_{i} {c}_{j} {m}_{i} {m}_{j}}$$

Two or more samples often are compared to visualize the effects of mappability. For example, sample CT407 (FIG. 2A, left) and CT410 (FIG. 2A, right) are plotted against each other on each axis in FIG. 2D. Points falling outside the diagonal in some aspects represent copy number differences between the two samples compared. Alternately or in combination, the above steps are performed without the aid of visualization, and instead stored on a non-transient computer medium. One skilled in the art will appreciate that alternative equations are also used to estimate copy number differences.

### Sequencing

Inputs, such as sequence read data, can be formatted in appropriate file formats. For example, sequence read data can be contained in FASTA files, FASTQ files, BAM files, SAM files, or other file formats. Input sequence read data can be unaligned. Input sequence read data can be aligned.

Sequence read data can be prepared for analysis. For example, reads can be trimmed for quality. Reads can also be trimmed to remove sequencing adapters, if necessary.

Sequence read data can be aligned. For example, read pairs can be aligned to a specified reference genome. In some cases, the reference genome is GRCh38. Alignment can be performed with a variety of algorithms or tools, including but not limited to SNAP, Burrows-Wheeler aligners (e.g., bwa-sw, bwa-mem, bwa-aln), Bowtie2, Novoalign, and modifications or variations thereof.

Quality control (QC) reports of the analysis can also be generated. QC reports can be used to identify failed libraries before conducting deeper sequencing. Such quality control reports can include a variety of metrics. QC metrics can include but are not limited to total read pairs, percent of duplicates (e.g., PCR duplicates), percent of unmapped reads, percent of reads with low map quality (e.g., Q<20), percent of read pairs mapped to different chromosomes, percent of read pair inserts (such as distance between mapping positions) between 0 and 1 kbp, percent of read pair inserts between 1 kbp and 100 kbp, percent of read pair inserts between 100 kbp and 1 Mbp, percent of read pair inserts above 1 Mbp, percent of read pairs containing a ligation junction, proximity to restriction fragment ends, a read pair separation plot, and an estimate of library complexity. QC metrics can be used to optimize the analysis, and to identify quality problems in reagents, samples, and users. Sequence alignments can be filtered based on one or more of the QC metrics. Duplicate reads can also be filtered, for example based on comparison of reads at closely corresponding positions.

Sequence read analysis results can include link density results. Link density results can include whole genome, one locus, and two locus views of link density results. Link density results can be output as a data set. Link density results can be presented as a linkage density plot (LDP), such as a heat map of interactions (e.g., contacts) between regions of a chromosome or a genome. Link density results can be associated with a score, such as a quality score. In some cases, link density visualizations are output for results that exceed a score threshold. In an example, visualizations are included for the whole genome, for de novo calls that exceed a score threshold, for single-sided candidate calls that exceed a score threshold and for all double-sided candidates, including those classified as negative. Link density visualization can include a scale (e.g., a color scale), a length scale bar, gene name labels, exon/intron structure glyphs for genes, and highlighting of detected rearrangements.

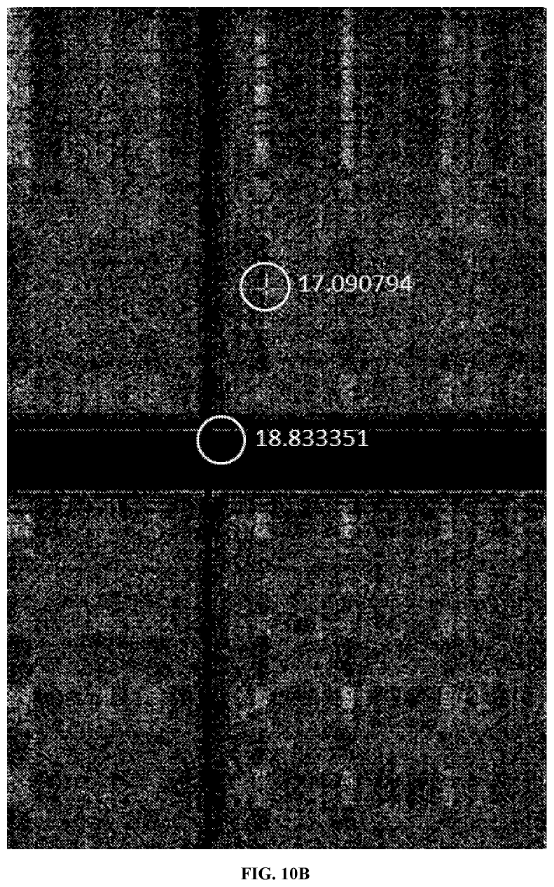

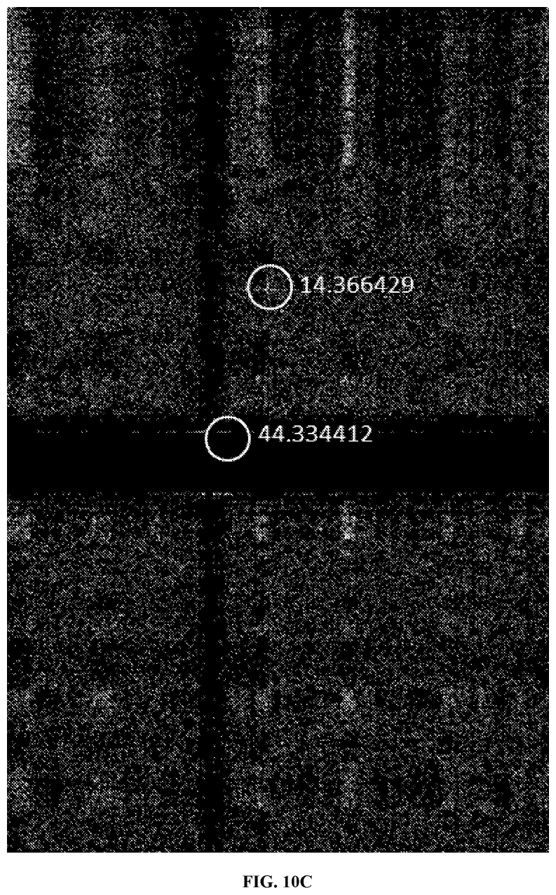

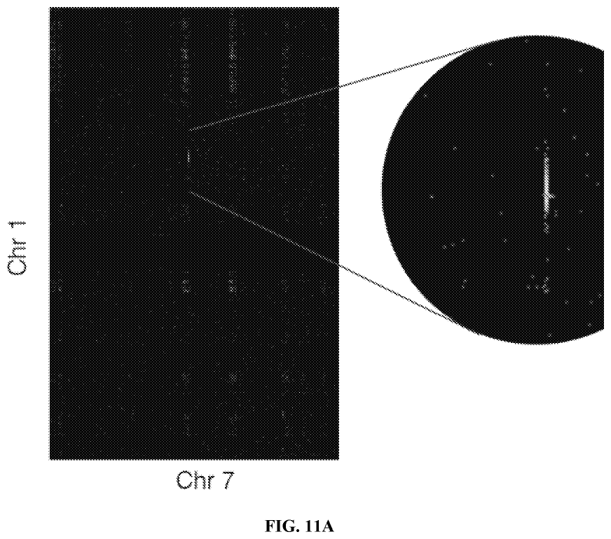

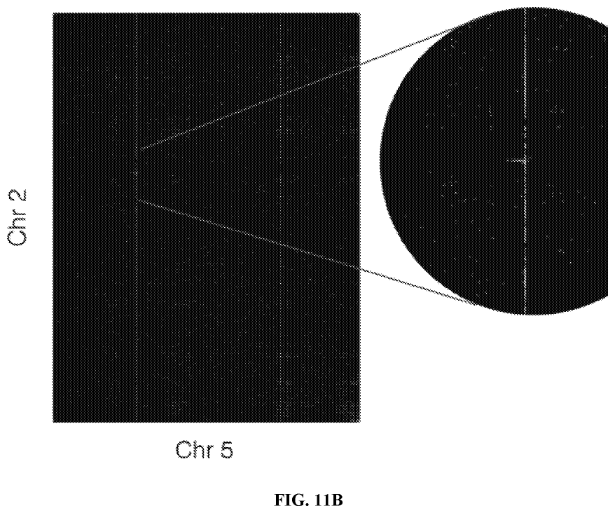

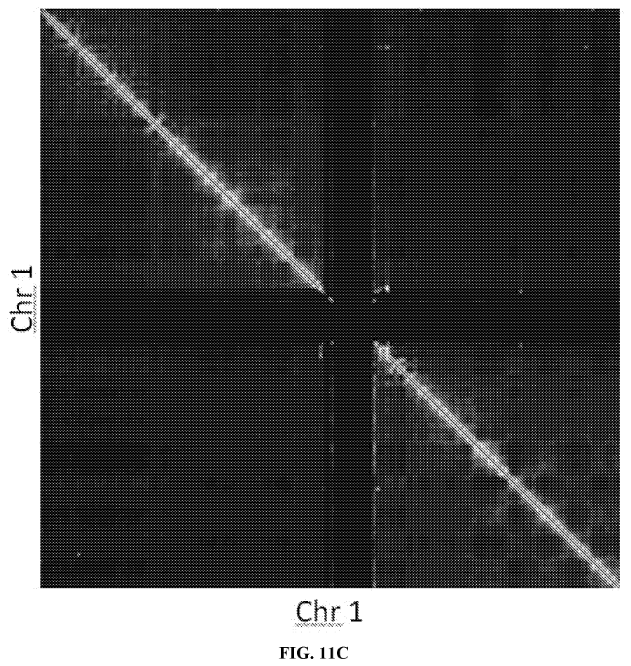

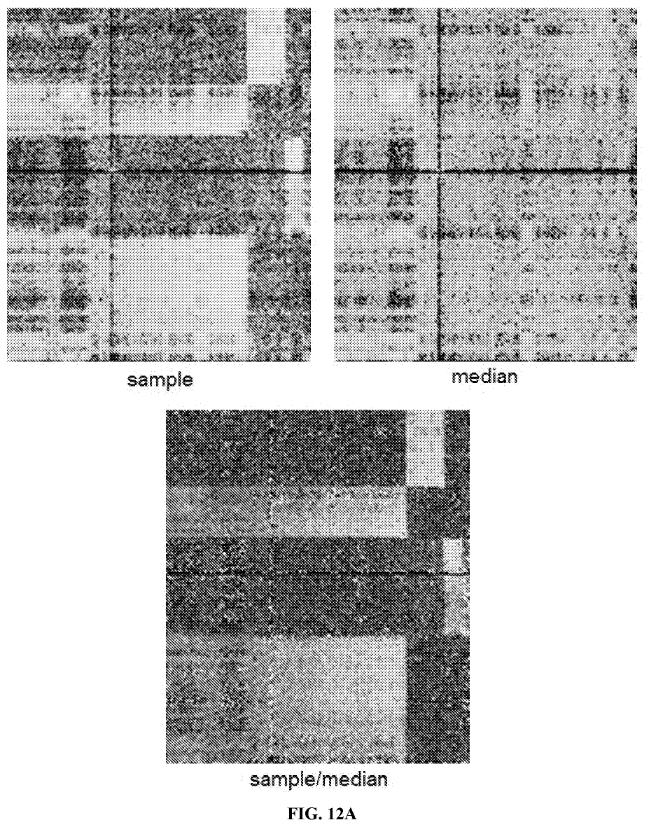

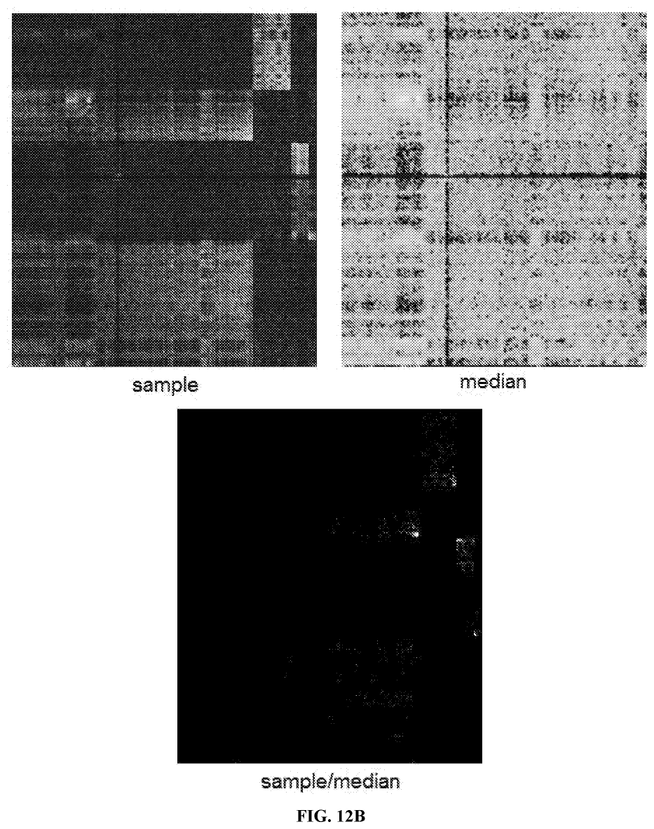

Linkage information can be normalized to control for effects and biases such as coverage, fragment mappability, fragment GC content, and fragment length. Normalization can be conducted by matrix balancing or other factor-agnostic methods. Matrix balancing can employ algorithms such as the Sinkhorn-Knopp algorithm or Knight-Ruiz normalization. Normalization can also be conducted to correct for background signal that may lead to false positives. For example, FIG. 10A, FIG. 10B, and FIG. 10C show image analysis-based results at the same pair of chromosomes compared in three different samples. Several “hits” (circled in the figures) are found in the same position across multiple samples, raising the suspicion that these are false positives. Normalization, such as by the median normalized read density across a pool of samples (e.g., 10 samples), can be used to correct individual sample data, for example by dividing the sample pixels by the median pixels. FIG. 11A, FIG. 11B, and FIG. 11C show median normalized read density (over 10 samples) for chromosome 1 versus chromosome 7 (FIG. 11A), chromosome 2 versus chromosome 5 (FIG. 11B), and chromosome 1 versus chromosome 1 (FIG. 11C). Normalization can be conducted with various bin handling approaches, including equal bin sizes, as shown in FIG. 12A, and with bin interpolation, as shown in FIG. 12B. In some cases, bin interpolation can yield reduced background noise compared to equal bin sizes, and result in more sharply resolved features.

Aligned sequence data can be analyzed for rearrangements, including rearrangements through the whole genome and rearrangements at specific two-locus (or two-sided) candidate genes. Analysis can also include identification of contacts, fusions, and joins. Alignments of sequence read data (e.g., in a BAM file or other suitable format) can be input into the analysis. Genome masking information can be input as well, or default genome masking information can be used in the analysis. Analysis can be conducted across the entire genome. Additionally or alternatively, analysis can be conducted for a list of two-sided candidate fusions. In some cases, the analysis conducted on a list of candidate fusions is more sensitive than the analysis conducted on a whole genome. Analysis of two-sided candidate fusions can detect fusions involving translocations of relatively short segments of DNA that may be missed by a genome-wide scan.

Distance measurements are made in some cases as combinations of base and base pairs. The minimum distance between breakpoints for detectable rearrangements can be less than, about, or a number in a range defined by two numbers selected from the list of nucleic acid lengths comprising 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 20 Mb, 30 Mb, 40 Mb, 50 Mb, 60 Mb, 70 Mb, 80 Mb, 90 Mb, 100 Mb, 200 Mb, 300 Mb, 400 Mb, 500 Mb, 600 Mb, 700 Mb, 800 Mb, 900 Mb, or 1 Gb.

Rearrangement analysis can produce a list of pairs of breakpoints that are deemed joined in the subject genome. The list of pairs of breakpoint coordinates can also include statistical significance or confidence metrics (e.g., p-value) for the breakpoint coordinate pairs. These pairs of breakpoints can be output in an appropriate format, such as browser extensible data (BED) or BED-PE.

Analysis of chromosome conformation can also be conducted using the techniques disclosed herein. For example, topologically associating domains (TADs) and TAD boundaries can be determined. Other topological domains and boundaries can also be determined, including but not limited to lamina-associated domains (LADs), replication time zones, and large organized chromatin K9-modification (LOCK) domains.

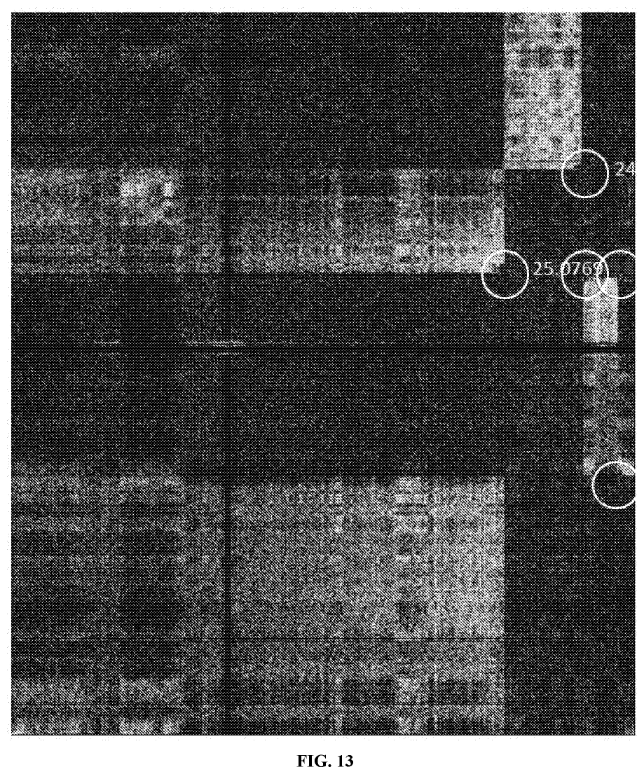

FIG. 13 shows analysis by a genome-wide scanning analysis pipeline. Sample calls made by the analytical pipeline are shown circled in white. FIG. 13 shows a plot of chromosome 3 versus chromosome 6, with 250 k bins.

In an exemplary embodiment, sequencing data is used to determine phasing information for polymorphisms known to be in the starting FFPE sample. For example, the sequencing data is used to determine whether certain polymorphisms such as SNPs were present on the same or different DNA molecules. Accuracy of the phasing determined using this method is measured by comparing to a known sequence, such as the sequence of the GIAB sample. For example, in some cases it is found that between 0-10,000, there were 132,796 SNPS found and 99.059% were in the correct phase. A high concordance (>95%) is seen up until about 1.5 MB (with the exception of the 70-80 kb bin, which missed 1 of 13 and the 1.1-1.3 MB bin which missed 2 of 15). In the 1.7-1.9 MB range, 7 of 7 SNP pair phases were properly called. From these data, it is concluded that, despite low levels of spurious linkage, proper long-range information is determined using the FFPE-Chicago method, even up to the megabase range. Importantly, these ‘concordance’ prediction rates are in many cases 95% or greater, significantly higher than the 50% success rate one would expect from random chance).

### Structural Phasing Information

Currently, structural and phasing analyses (e.g., for medical purposes) remain challenging. For example, there is astounding heterogeneity among cancers, individuals with the same type of cancer, or even within the same tumor. Teasing out the causative from consequential effects can require very high precision and throughput at a low per-sample cost. In the domain of personalized medicine, one of the gold standards of genomic care is a sequenced genome with all variants thoroughly characterized and phased, including large and small structural rearrangements and novel mutations. To achieve this with previous technologies demands effort akin to that required for a de novo assembly, which is currently too expensive and laborious to be a routine medical procedure.

Phasing information includes maternal/paternal phasing as well as tumor/non-tumor phasing information. Tumor/non-tumor phasing can be used to differentiate cancer genomic information from somatic genomic information.

In some embodiments of the disclosure, a preserved tissue (e.g., an FFPE tissue) from a subject can be provided and the method can return an assembled genome, alignments with called variants (including large structural variants and copy number variants), phased variant calls, or any additional analyses. In other embodiments, the methods disclosed herein can provide long distance read pair libraries directly for the individual.

In various embodiments of the disclosure, the methods disclosed herein can generate long-range read pairs separated by large distances. The upper limit of this distance may be improved by the ability to collect DNA samples of large size. In some cases, the read pairs can span up to 50, 60, 70, 80, 90, 100, 125, 150, 175, 200, 225, 250, 300, 400, 500, 600, 700, 800, 900, 1000, 1500, 2000, 2500, 3000, 4000, 5000 kbp or more in genomic distance. In some examples, the read pairs can span up to 500 kbp in genomic distance. In other examples, the read pairs can span up to 2000 kbp in genomic distance. The methods disclosed herein can integrate and build upon standard techniques in molecular biology, and are further well-suited for increases in efficiency, specificity, and genomic coverage.

In other embodiments, the methods disclosed herein can be used with currently employed sequencing technology. For example, the methods can be used in combination with well-tested and/or widely deployed sequencing instruments. In further embodiments, the methods disclosed herein can be used with technologies and approaches derived from currently employed sequencing technology.

In various embodiments, the disclosure provides for one or more methods disclosed herein that comprise the step of probing the physical layout of chromosomes within preserved (e.g., FFPE) samples or cells. Examples of techniques to probe the physical layout of chromosomes through sequencing include the “C” family of techniques, such as chromosome conformation capture (“3C”), circularized chromosome conformation capture (“4C”), carbon-copy chromosome capture (“5C”), and Hi-C based methods; and ChIP based methods, such as ChIP-loop, ChIP-PET. These techniques utilize the fixation of chromatin in live cells to cement spatial relationships in the nucleus. Subsequent processing and sequencing of the products allows a researcher to recover a matrix of proximate associations among genomic regions. With further analysis these associations can be used to produce a three-dimensional geometric map of the chromosomes as they are physically arranged in the preserved (e.g., FFPE) sample. Such techniques describe the discrete spatial organization of chromosomes, and provide an accurate view of the functional interactions among chromosomal loci.

In some embodiments, the intrachromosomal interactions correlate with chromosomal connectivity. In some cases, the intrachromosomal data can aid genomic assembly. In some cases, the chromatin is reconstructed in vitro. This can be advantageous because chromatin—particularly histones, the major protein component of chromatin—is important for fixation under the most common “C” family of techniques for detecting chromatin conformation and structure through sequencing: 3C, 4C, 5C, and Hi-C. Chromatin is highly non-specific in terms of sequence and will generally assemble uniformly across the genome. In some cases, the genomes of species that do not use chromatin can be assembled on a reconstructed chromatin and thereby extend the horizon for the disclosure to all domains of life.

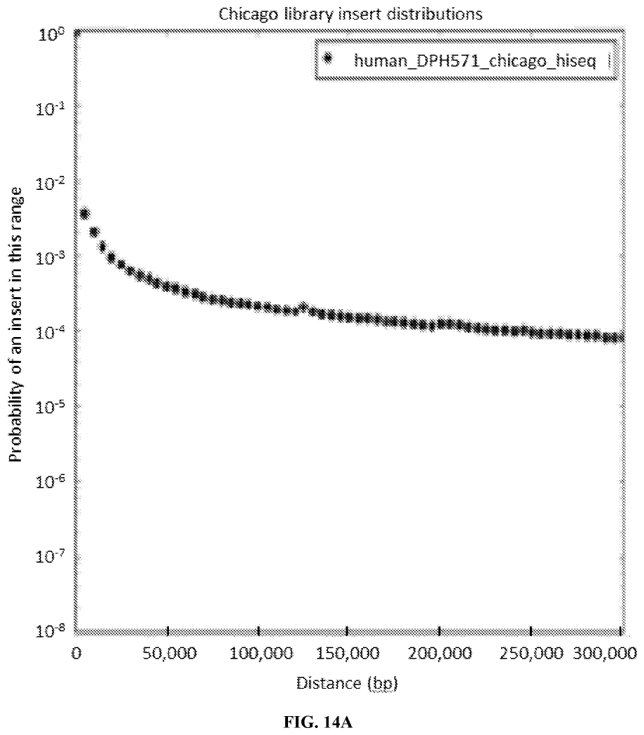

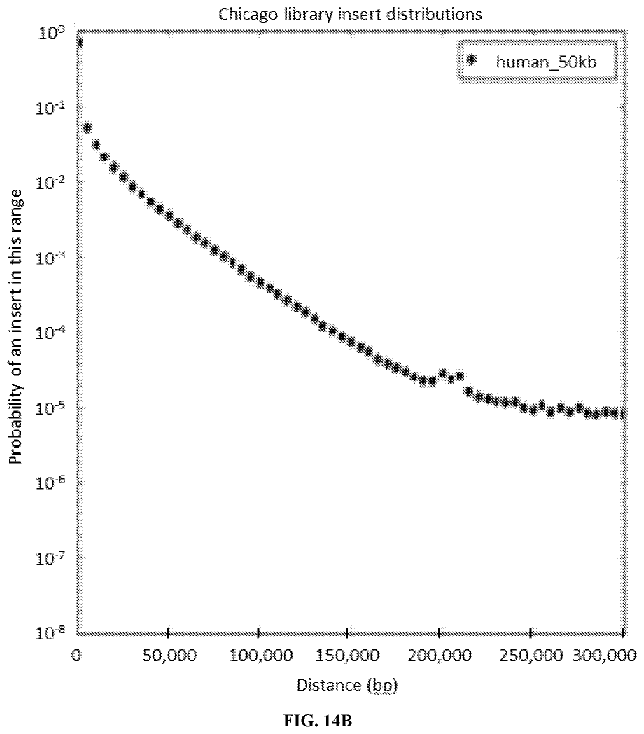

Read pair data can be obtained from a chromatin conformation capture technique. In some examples, ligation or other tagging is accomplished so as to mark genome regions that are in close physical proximity. Crosslinking of the complex such that proteins (such as histones) are stably bound in a complex with the DNA molecule, e.g. genomic DNA, within chromatin can be accomplished according to a suitable method described in further detail elsewhere herein or otherwise known in the art. In some cases, crosslinks arising from sample preservation (e.g., from fixation) are utilized by extracting DNA-protein complexes under conditions such that such complexes are not degraded, such as through the exclusion of proteinase K treatment. For example, nucleotide segments that are not in close proximity along a genome sequence can be in close physical proximity when part of a structure such as chromatin. Such nucleotide segments can be ligated together and subsequently analyzed according to methods of the present disclosure. For example, ligated nucleotide segments can be sequenced and the distance between the sequenced ends of two ligated segments (insert distance) can be analyzed. FIG. 14A shows a graph of the probability of an insert in a particular range as a function of insert distance in base pairs (bp) for a preserved sample (e.g., an FFPE sample) analyzed by techniques of the present disclosure. FIG. 14B shows a similar graph for a sample analyzed using a Chicago method. In both graphs, the x-axis shows the insert distance (bp), from 0 to 300,000, while the y-axis shows the probability of an insert of that distance, from 10⁰ at the top of the axis to 10⁻⁸ at the bottom of the axis (logarithmic).

In some cases, two or more nucleotide sequences can be crosslinked via proteins bound to one or more nucleotide sequences. One approach is to expose the chromatin to ultraviolet irradiation (Gilmour et al., Proc. Nat'l. Acad. Sci. USA 81:4275-4279, 1984). Crosslinking of polynucleotide segments may also be performed utilizing other approaches, such as chemical or physical (e.g. optical) crosslinking. Suitable chemical crosslinking agents include, but are not limited to, formaldehyde and psoralen (Solomon et al., Proc. Natl. Acad. Sci. USA 82:6470-6474, 1985; Solomon et al., Cell 53:937-947, 1988). For example, crosslinking can be performed by adding 2% formaldehyde to a mixture comprising the DNA molecule and chromatin proteins. Other examples of agents that can be used to crosslink DNA include, but are not limited to, UV light, mitomycin C, nitrogen mustard, melphalan, 1,3-butadiene diepoxide, cis diaminedichloroplatinum(II) and cyclophosphamide. Suitably, the crosslinking agent will form crosslinks that bridge relatively short distances—such as about 2 Å—thereby selecting intimate interactions that can be reversed.

Universally, procedures for probing the physical layout of chromosomes, such as Hi-C based techniques, utilize chromatin that is formed within a cell/organism, such as chromatin isolated from cultured cells or primary tissue. Chicago based methods provide not only for the use of such techniques with chromatin isolated from a cell/organism but also with reconstituted chromatin. Reconstituted chromatin is differentiated from chromatin formed within a cell/organism over various features. First, for many samples, the collection of naked DNA samples can be achieved by using a variety of noninvasive to invasive methods, such as by collecting bodily fluids, swabbing buccal or rectal areas, taking epithelial samples, etc. Second, reconstituting chromatin substantially prevents the formation of inter-chromosomal and other long-range interactions that generate artifacts for genome assembly and haplotype phasing. In some cases, a sample may have less than about 20, 15, 12, 11, 10, 9, 8, 7, 6, 5, 4, 3, 2, 1, 0.5, 0.4, 0.3, 0.2, 0.1% or less inter-chromosomal or intermolecular crosslinking according to the methods and compositions of the disclosure. In some examples, the sample may have less than about 5% inter-chromosomal or intermolecular crosslinking. In some examples, the sample may have less than about 3% inter-chromosomal or intermolecular crosslinking. In further examples, may have less than about 1% inter-chromosomal or intermolecular crosslinking. Third, the frequency of sites that are capable of crosslinking and thus the frequency of intramolecular crosslinks within the polynucleotide can be adjusted. For example, the ratio of DNA to histones can be varied, such that the nucleosome density can be adjusted to a desired value. In some cases, the nucleosome density is reduced below the physiological level. Accordingly, the distribution of crosslinks can be altered to favor longer-range interactions. In some embodiments, sub-samples with varying crosslinking density may be prepared to cover both short- and long-range associations. For example, the crosslinking conditions can be adjusted such that at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 40%, about 45%, about 50%, about 60%, about 70%, about 80%, about 90%, about 95%, or about 100% of the crosslinks occur between DNA segments that are at least about 50 kb, about 60 kb, about 70 kb, about 80 kb, about 90 kb, about 100 kb, about 110 kb, about 120 kb, about 130 kb, about 140 kb, about 150 kb, about 160 kb, about 180 kb, about 200 kb, about 250 kb, about 300 kb, about 350 kb, about 400 kb, about 450 kb, or about 500 kb apart on the sample DNA molecule.

High degrees of accuracy required by cancer genome sequencing can be achieved using the methods and systems described herein. Inaccurate reference genomes can make base-calling challenging when sequencing cancer genomes. Heterogeneous samples and small starting materials, for example a sample obtained by biopsy introduce additional challenges. Further, detection of large scale structural variants and/or losses of heterozygosity is often crucial for cancer genome sequencing, as well as the ability to differentiate between somatic variants and errors in base-calling.

Systems and methods described herein may generate accurate long sequences from complex samples containing 2, 3, 4, 5, 6, 7, 8, 9, 10, 12, 15, 20 or more varying genomes. Mixed samples of normal, benign, and/or tumor origin may be analyzed, optionally without the need for a normal control. In some embodiments, starting samples as little as 100 ng or even as little as hundreds of genome equivalents are utilized to generate accurate long sequences. Systems and methods described herein may allow for detection of copy number variants, large scale structural variants and rearrangements, phased variant calls may be obtained over long sequences spanning about 1 kbp, about 2 kbp, about 5 kbp, about 10 kbp, 20 kbp, about 50 kbp, about 100 kbp, about 200 kbp, about 500 kbp, about 1 Mbp, about 2 Mbp, about 5 Mbp, about 10 Mbp, about 20 Mbp, about 50 Mbp, or about 100 Mbp or more nucleotides. For example, phase variant calls may be obtained over long sequences spanning about 1 Mbp or about 2 Mbp.

Haplotypes determined using the methods and systems described herein may be assigned to computational resources, for example computational resources over a network, such as a cloud system. Short variant calls can be corrected, if necessary, using relevant information that is stored in the computational resources. Structural variants can be detected based on the combined information from short variant calls and the information stored in the computational resources. Problematic parts of the genome, such as segmental duplications, regions prone to structural variation, the highly variable and medically relevant MHC region, centromeric and telomeric regions, and other heterochromatic regions including but limited to those with repeat regions, low sequence accuracy, high variant rates, ALU repeats, segmental duplications, or any other relevant problematic parts known in the art, can be reassembled for increased accuracy.

A sample type can be assigned to the sequence information either locally or in a networked computational resource, such as a cloud. In cases where the source of the information is known, for example when the source of the information is from a cancer or normal tissue, the source can be assigned to the sample as part of a sample type. Other sample type examples generally include, but are not limited to, tissue type, sample collection method, presence of infection, type of infection, processing method, size of the sample, etc. In cases where a complete or partial comparison genome sequence is available, such as a normal genome in comparison to a cancer genome, the differences between the sample data and the comparison genome sequence can be determined and optionally output.

### Methods for Haplotype Phasing

Because the read pairs generated by the methods disclosed herein are generally derived from intra-chromosomal contacts, any read pairs that contain sites of heterozygosity will also carry information about their phasing. Using this information, reliable phasing over short, intermediate and even long (megabase) distances can be performed rapidly and accurately. Experiments designed to phase data from one of the 1000 genomes trios (a set of mother/father/offspring genomes) have reliably inferred phasing. Additionally, haplotype reconstruction using proximity-ligation similar to Selvaraj et al. (Nature Biotechnology 31:1111-1118 (2013)) can also be used with haplotype phasing methods disclosed herein.

For example, a haplotype reconstruction using proximity-ligation based method can also be used in the methods disclosed herein in phasing a genome. A haplotype reconstruction using proximity-ligation based method combines a proximity-ligation and DNA sequencing with a probabilistic algorithm for haplotype assembly. First, proximity-ligation sequencing is performed using a chromosome capture protocol, such as the Hi-C protocol. These methods can capture DNA fragments from two distant genomic loci that looped together in three-dimensional space. After shotgun DNA-sequencing of the resulting DNA library, paired-end sequencing reads have ‘insert sizes’ that range from several hundred base pairs to tens of millions of base pairs. Thus, short DNA fragments generated in a Hi-C experiment can yield small haplotype blocks, long fragments ultimately can link these small blocks together. With enough sequencing coverage, this approach has the potential to link variants in discontinuous blocks and assemble every such block into a single haplotype. This data is then combined with a probabilistic algorithm for haplotype assembly. The probabilistic algorithm utilizes a graph in which nodes correspond to heterozygous variants and edges correspond to overlapping sequence fragments that may link the variants. This graph might contain spurious edges resulting from sequencing errors or trans interactions. A max-cut algorithm is then used to predict parsimonious solutions that are maximally consistent with the haplotype information provided by the set of input sequencing reads. Because proximity ligation generates larger graphs than conventional genome sequencing or mate-pair sequencing, computing time and number of iterations are modified so that the haplotypes can be predicted with reasonable speed and high accuracy. The resulting data can then be used to guide local phasing using Beagle software and sequencing data from the genome project to generate chromosome-spanning haplotypes with high resolution and accuracy.

Determining Phase Information with Paired Ends

Further provided herein are methods and compositions for determining phase information from paired ends derived from FFPE-samples. Paired ends can be generated by any of the methods disclosed or those further illustrated in the provided Examples. For example, in the case of a DNA molecule bound to a solid surface which was subsequently cleaved, following re-ligation of free ends, re-ligated DNA segments are released from the solid-phase attached DNA molecule, for example, by restriction digestion. This release results in a plurality of paired end fragments. In some cases, the paired ends are ligated to amplification adapters, amplified, and sequenced with short read technology. In these cases, paired ends from multiple different solid phase-bound DNA molecules are within the sequenced sample. However, it is confidently concluded that for either side of a paired end junction, the junction adjacent sequence is derived from a common phase of a common molecule. In cases where paired ends are linked with a punctuation oligonucleotide, the paired end junction in the sequencing read is identified by the punctuation oligonucleotide sequence. In other cases, the pair ends were linked by modified nucleotides, which can be identified based on the sequence of the modified nucleotides used.

Alternatively, following release of paired ends, the free paired ends are ligated to amplification adapters and amplified. In these cases, the plurality of paired ends is then bulk ligated together to generate long molecules which are read using long-read sequencing technology. In other examples, released paired ends are bulk ligated to each other without the intervening amplification step. In either case, the embedded read pairs are identifiable via the native DNA sequence adjacent to the linking sequence, such as a punctuation sequence or modified nucleotides. The concatenated paired ends are read on a long-sequence device, and sequence information for multiple junctions is obtained. Since the paired ends derived from multiple different solid phase-bound DNA molecules, sequences spanning two individual paired ends, such as those flanking amplification adapter sequences, are found to map to multiple different DNA molecules. However, it is confidently concluded that for either side of a paired end junction, the junction-adjacent sequence is derived from a common phase of a common molecule. For example, in the case of paired ends derived from a punctuated molecule, sequences flanking the punctuation sequence are confidently assigned to a common DNA molecule. In preferred cases, because the individual paired ends are concatenated using the methods and compositions disclosed herein, one is able to sequence multiple paired ends in a single read.

Sequencing data generated using the methods and compositions described herein are used, in preferred embodiments, to generate phased de novo sequence assemblies, determine phase information, and/or identify structural variations.

### Determining Structural Variations and Other Genetic Features

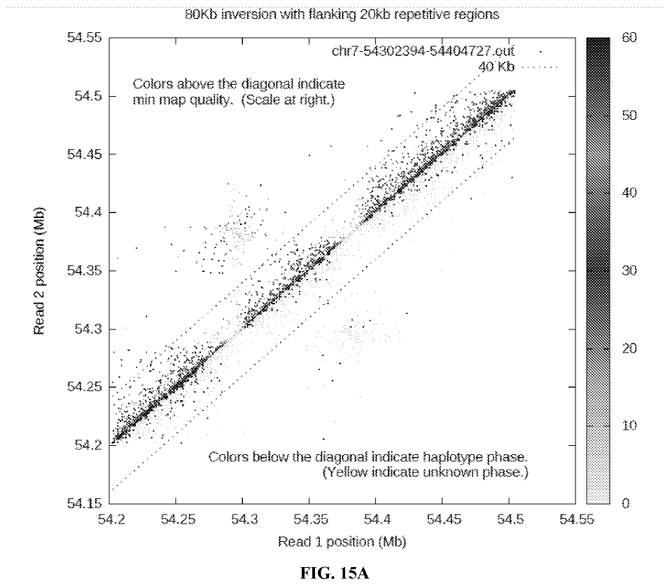

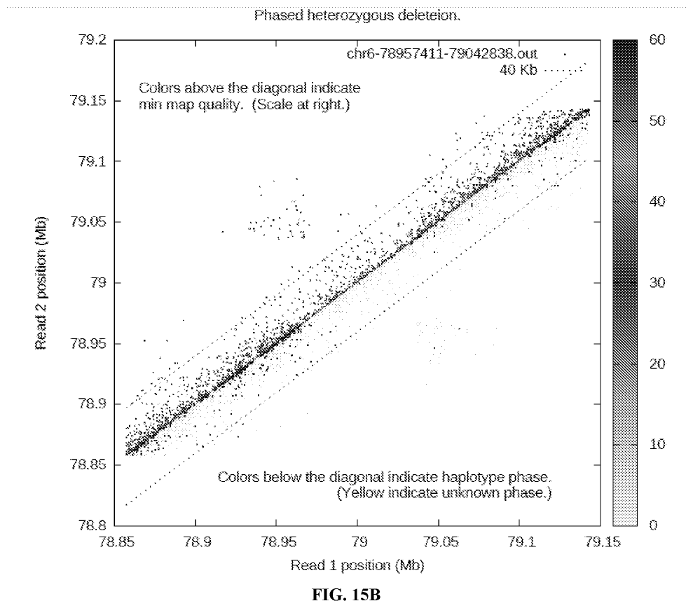

Referring to FIG. 15A and FIG. 15B, an example is provided of mapped locations on a reference sequence, e.g., GRCh38, of read pairs generated from proximity ligation of DNA from re-assembled chromatin are plotted in the vicinity of structural differences between GM12878 and the reference. Each read pair generated is represented both above and below the diagonal. Above the diagonal, shades indicates map quality score on scale shown; below the diagonal shades indicate the inferred haplotype phase of generated read pairs based on overlap with a phased SNPs. In some embodiments, plots generated depict inversions with flanking repetitive regions, as illustrated in FIG. 15B. In some embodiments, plots generated depict data for a phased heterozygous deletion, as illustrated in FIG. 15B.

Mapping paired sequence reads from one individual against a reference is the most commonly used sequence-based method for identifying differences in contiguous nucleic acid or genome structure like inversions, deletions and duplications (Tuzun et al., 2005). FIG. 15A and FIG. 15B show how read pairs generated by proximity ligation of DNA from re-assembled chromatin from GM12878 mapped to the human reference genome GRCh38 reveal two such structural differences. To estimate the sensitivity and specificity of the read pair data for identifying structural differences, a maximum likelihood discriminator on simulated data sets constructed to simulate the effect of heterozygous inversions was tested. The test data was constructed by randomly selecting intervals of a defined length L from the mapping of the NA12878 reads generated to the GRCh38 reference sequence and assigning each generated read pair independently at random to the inverted or reference haplotype, and editing the mapped coordinates accordingly. Non-allelic homologous recombination is responsible for much of the structural variation observed in human genomes, resulting in many variation breakpoints that occur in long blocks of repeated sequence (Kidd et al., 2008). The effect of varying lengths of repetitive sequence surrounding the inversion breakpoints was simulated by removing all reads mapped to within a distance W of them. In the absence of repetitive sequences at the inversion breakpoints, for 1 Kbp, 2 Kbp and 5 Kbp inversions respectively, the sensitivities (specificities) were 0.76 (0.88), 0.89 (0.89) and 0.97 (0.94) respectively. When 1 Kbp regions of repetitive (unmappable) sequence at the inversion breakpoints was used in a simulation, the sensitivity (specificity) for 5 Kbp inversions was 0.81 (0.76).

### Performance

Analysis conducted with the techniques disclosed herein can be performed at high accuracy. Analysis can be conducted with an accuracy of at least about 50%, 60%, 70%, 75%, 80%, 85%, 90%, 95%, 96%, 97%, 98%, 99%, 99.9%, 99.99%, 99.999% or more. Analysis can be conducted with an accuracy of at least 70%. Analysis can be conducted with an accuracy of at least 80%. Analysis can be conducted with an accuracy of at least 90%.

Analysis conducted with the techniques disclosed herein can be performed at high specificity. Analysis can be conducted with a specificity of at least about 50%, 60%, 70%, 75%, 80%, 85%, 90%, 95%, 96%, 97%, 98%, 99%, 99.9%, 99.99%, 99.999% or more. Analysis can be conducted with a specificity of at least 70%. Analysis can be conducted with a specificity of at least 80%. Analysis can be conducted with a specificity of at least 90%.

Analysis conducted with the techniques disclosed herein can be performed at high sensitivity. Analysis can be conducted with a sensitivity of at least about 50%, 60%, 70%, 75%, 80%, 85%, 90%, 95%, 96%, 97%, 98%, 99%, 99.9%, 99.99%, 99.999% or more. Analysis can be conducted with a sensitivity of at least 70%. Analysis can be conducted with a sensitivity of at least 80%. Analysis can be conducted with a sensitivity of at least 90%.

Use of the techniques of the present disclosure can improve the functioning of the computer systems on which they are implemented. For example, the techniques can reduce the processing time for a given analysis by at least about 5%, 10%, 15%, 20%, 25%, 30%, 35%, 40%, 45%, 50%, 55%, 60%, 65%, 70%, 75%, 80%, 85%, 90%, 95%, or more. The techniques can reduce the memory requirements for a given analysis by at least about 5%, 10%, 15%, 20%, 25%, 30%, 35%, 40%, 45%, 50%, 55%, 60%, 65%, 70%, 75%, 80%, 85%, 90%, 95%, or more.

Use of the techniques of the present disclosure can enable conducting analyses that were previously not possible. For example, certain genetic features can be detected from sequence information that would not be detectable from such information without the methods of the present disclosure.

### Machine Learning

Analysis to identify features such as contacts and rearrangements (including but not limited to deletions, duplications, insertions, inversions or reversals, translocations, joins, fusions, and fissions), and other interactions can be conducted with a variety of techniques. Analysis techniques can include statistical and probability analysis, signal processing including Fourier analysis, computer vision and other image processing, language processing (e.g., natural language processing), and machine learning. For example, interaction plots such as contact matrixes can be analyzed for data configurations indicative of features such as those above. In some cases, filters can be applied to plots or other data. Filters can be convolution filters including but not limited to smoothing filters (e.g., kernel smoothing or Savitzky-Golay filter, Gaussian blur, among others).

Some embodiments involve machine learning as a component of genome structure determination, and accordingly some computer systems are configured to comprise a module having a machine learning capacity. Machine learning modules comprise at least one of the following listed modalities, so as to constitute a machine learning functionality.

Modalities that constitute machine learning variously demonstrate a data filtering capacity, so as to be able to perform automated mass spectrometric data spot detection and calling. This modality is in some cases facilitated by the presence of predicted patterns indicative of various genomic structural changes, such as inversions, insertions, deletions, or translocations.

Modalities that constitute machine learning variously demonstrate a data treatment or data processing capacity, so as to render read pair frequencies in a form conducive to downstream analysis. Examples of data treatment include but are not necessarily limited to log transformation, assigning of scaling ratios, or mapping data to crafted features so as to render the data in a form that is conducive to downstream analysis.

Machine learning data analysis components as disclosed herein regularly process a wide range of features in a read pair data set, such as 1 to 10,000 features, or 2 to 300,000 features, or a number of features within either of these ranges or higher than either of these ranges. In some cases, data analysis involves at least 1 k, 2 k, 3 k, 4 k, 5 k, 6 k, 7 k, 8 k, 9 k, 10 k, 20 k, 30 k, 40 k, 50 k, 60 k, 70 k, 80 k, 90 k, 100 k, 120 k, 140 k, 160 k, 180 k, 200 k, 220 k, 2240 k, 260 k, 280 k, 300 k, or more than 300 k features.

Read pair distribution patterns are identified using any number of approaches consistent with the disclosure herein. In some cases, read pair distribution patterns selection comprises elastic net, information gain, random forest imputing or other feature selection approaches consistent with the disclosure herein and familiar to one of skill in the art.

Selected read pair distribution patterns are matched against predicted patterns indicative of a genomic structural change, again using any number of approaches consistent with the disclosure herein. In some cases, read pair pattern detection comprises logistic regression, SVM, random forest, KNN, or other classifier approaches consistent with the disclosure herein and familiar to one of skill in the art.

Applying machine learning, or providing a machine learning module on a computer configured for the analyses disclosed herein, allows for the detection of relevant genomic structural changes for asymptomatic disease detection or early detection as part of an ongoing monitoring procedure, so as to identify a disease or disorder either ahead of symptom development or while intervention is either more easily accomplished or more likely to bring about a successful outcome.

Applying machine learning, or providing a machine learning module on a computer configured for the analyses disclosed herein also allows identification of structural rearrangements in individuals subjected to a drug treatment, for example as part of a drug trial, so that outcome of the trial for the individual or for the population may be concurrently or retrospectively correlated so as to identify particular genomic structural events that correspond positively or negatively with drug efficacy.

Applying machine learning, or providing a machine learning module on a computer configured for the analyses disclosed herein also allows identification of structural rearrangements that correspond with particular regions of genetically heterogeneous samples, such as tumor tissue samples collected without homogenization so as to preserve positional information in the sample. As some tumor regions are known to correspond to cell populations particularly adept at metastasis or tumor spread, identifying genomic rearrangements or other phase information that correlates with such cell populations assists in selecting a treatment regimen to target these particularly dangerous cell populations.

Monitoring is often but not necessarily performed in combination with or in support of a genetic assessment indicating a genetic predisposition for a disorder for which a signature of onset or progression is monitored. Similarly, in some cases machine learning is used to facilitate monitoring of or assessment of treatment efficacy for a treatment regimen, such that the treatment regimen can be modified over time, continued or resolved as indicated by the ongoing proteomics mediated monitoring.

Machine learning approaches and computer systems having modules configured to execute machine learning algorithms facilitate identification of phase information or genomic rearrangement in datasets of varying complexity. In some cases the phase information or genomic rearrangements are identified from an untargeted database comprising a large amount of mass spectrometric data, such as data obtained from a single individual at multiple time points, samples taken from multiple individuals such as multiple individuals of a known status for a condition of interest or known eventual treatment outcome or response, or from multiple time points and multiple individuals.

Alternately, in some cases machine learning facilitates the refinement of a genomic rearrangement or phase information through the analysis of a database targeted to that a genomic rearrangement or phase information, by for example collecting a genomic rearrangement or phase information from a single individual over multiple time points, when a health condition for the individual is known for the time points, or collecting sequence information from multiple individuals of known status for a condition of interest, or collecting sequence information from multiple individuals at multiple time points. As is readily apparent, in some cases collection of sequence information is facilitated through the use of preserved sample such as crosslinked samples collected pursuant to surgery or FFPE samples collected pursuant to a drug trial.

Thus, sequence information is collected either alone or in combination with drug trial outcome or surgical intervention outcome information. Sequence data is subjected to machine learning, for example on a computer system configured as disclosed herein, so as to identify a subset of read pairs indicative of a pattern corresponding to a genomic rearrangement that either alone or in combination with one or more additional markers, account for a health status signal. Thus, machine learning in some cases facilitates identification of sequence, either DNA or RNA sequence, or of a genomic rearrangement that is individually informative of a health status in an individual.

An example machine learning approach consistent with the above disclosure is Convolution Neural Network (CNN). CNN is useful for, for example, classifying positive and negative samples. Exemplary CNN architecture contains 2 fully connected convolutional hidden layers each followed by a max-pooling layer and final output layer of a number of neurons such as a number of neurons divisible only by two or factors of two, such as 128, 256, 512, 1024, or other numbers of neurons with logit activation function. In alternate embodiments, a wide range of neuron numbers are compatible with disclosures herein, such a number in a range defined by endpoints varying from less than 50, 50, 60, 64, 70, 80, 90, 100, 120, 140, 160, 180, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2048, 2100, 2200, 2300, 2400, 2500, 2600, 2700, 2800, 2900, 3000, or greater than 3000.

From some implementations of machine learning such as CNN, training data uses read-pair count information and an intra chromosomal matrix is normalized using, for example, the inverse of distance from the diagonal to the read pair mapping point. Alternately or in combination, other parameters such as reference mappability, restriction site distribution or others are used as additional channels to create multi-channel neural networks such as CNN network.

Image classification is implemented using feature localization through a number of state of the art networks such as YOLO, Mask R-CNN, Fast R-CNN, among other approaches. Alternately, specifically tailored domain architectures are designed for a particular application.

### Computer Systems

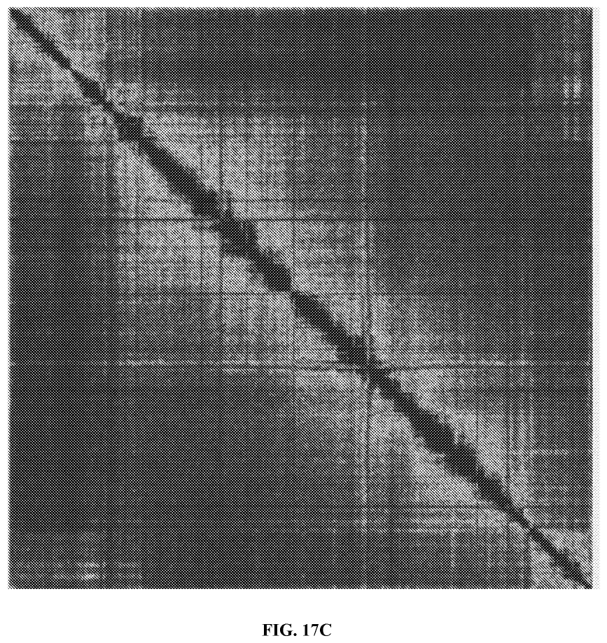

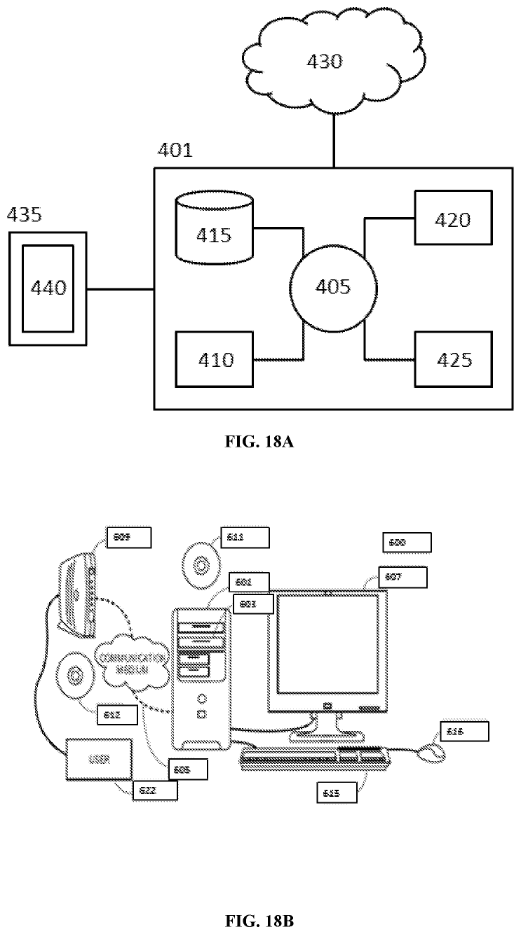

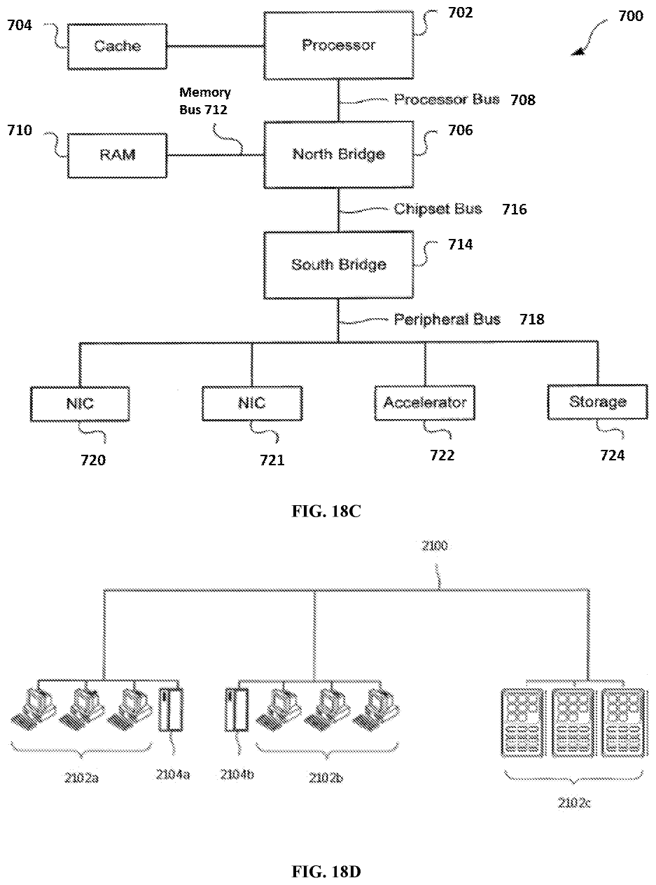

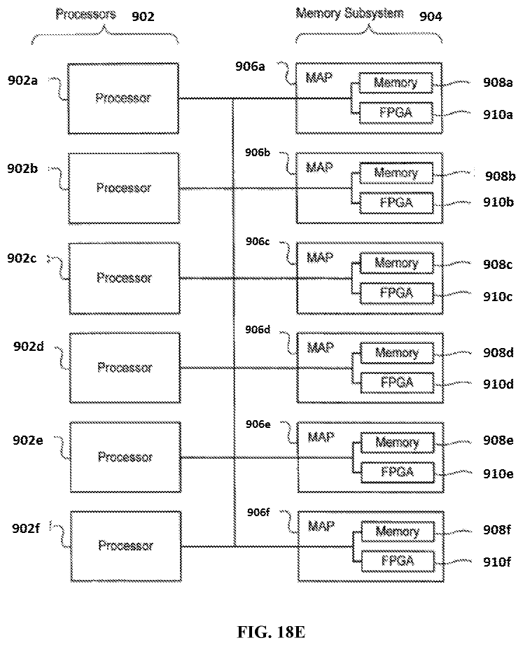

FIG. 18A shows a computer system 401 that is programmed or otherwise configured to implement the methods provided herein. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software.

During use, the code can be executed by the processor 405. In some cases, the code can be retrieved from the storage unit 415 and stored on the memory 410 for ready access by the processor 1005. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, an output or readout of the trained algorithm. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 405.

Computer systems herein are in some cases configured to execute machine learning operations such as those disclosed in the specification herein or otherwise known to one of skill in the art.

The computer system 600 illustrated in FIG. 18B may be understood as a logical apparatus that can read instructions from media 611 and/or a network port 605, which can optionally be connected to server 609 having fixed media 612. The system, such as shown in FIG. 18B can include a CPU 601, disk drives 603, optional input devices such as keyboard 615 and/or mouse 616 and optional monitor 607. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 622 as illustrated in FIG. 18B.

FIG. 18C is a block diagram illustrating a first example architecture of a computer system 700 that can be used in connection with example embodiments described herein. As depicted in FIG. 18C, the example computer system includes a processor 702 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some embodiments, multiple processors or processors with multiple cores are used, whether in a single computer system, in a cluster, or distributed across systems over a network comprising a plurality of computers, cell phones, and/or personal data assistant devices.

As illustrated in FIG. 18C, a high speed cache 704 can be connected to, or incorporated in, the processor 702 to provide a high speed memory for instructions or data that have been recently, or are frequently, used by processor 702. The processor 702 is connected to a north bridge 706 by a processor bus 708. The north bridge 706 is connected to random access memory (RAM) 710 by a memory bus 712 and manages access to the RAM 710 by the processor 702. The north bridge 706 is also connected to a south bridge 714 by a chipset bus 716. The south bridge 714 is, in turn, connected to a peripheral bus 718. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 718. In some alternative architectures, the functionality of the north bridge can be incorporated into the processor instead of using a separate north bridge chip.

In some embodiments, system 700 includes an accelerator card 722 attached to the peripheral bus 718. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 724 and can be loaded into RAM 710 and/or cache 704 for use by the processor. The system 2000 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention.

In this example, system 700 also includes network interface cards (NICs) 720 and 721 connected to the peripheral bus for providing network interfaces to external storage, such as Network Attached Storage (NAS) and other computer systems that can be used for distributed parallel processing.

FIG. 18D is a diagram showing a network 2100 with a plurality of computer systems 2102a, and 2102b, a plurality of cell phones and personal data assistants 2102c, and Network Attached Storage (NAS) 2104a, and 2104b. In example embodiments, systems 2102a, 2102b, and 2102c can manage data storage and optimize data access for data stored in Network Attached Storage (NAS) 2104a and 2104b. A mathematical model can be used for the data and be evaluated using distributed parallel processing across computer systems 2102a, and 2102b, and cell phone and personal data assistant systems 2102c. Computer systems 2102a, and 2102b, and cell phone and personal data assistant systems 2102c can also provide parallel processing for adaptive data restructuring of the data stored in Network Attached Storage (NAS) 2104a and 2104b. FIG. 18D illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane or other connectors for parallel processing by other processors. In other embodiments, some or all of the processors can use a shared virtual address memory space.

FIG. 18E is a block diagram of a multiprocessor computer system 900 using a shared virtual address memory space in accordance with an example embodiment. The system includes a plurality of processors 902a-f that can access a shared memory subsystem 904. The system incorporates a plurality of programmable hardware memory algorithm processors (MAPs) 906a-f in the memory subsystem 904. Each MAP 906a-f can comprise a memory 908a-f and one or more field programmable gate arrays (FPGAs) 910a-f. The MAP provides a configurable functional unit and particular algorithms or portions of algorithms can be provided to the FPGAs 910a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 908a-f, allowing it to execute tasks independently of, and asynchronously from, the respective microprocessor 902a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors, FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays, Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs) as referenced in FIG. 18E, system on chips (SOCs), application specific integrated circuits (ASICs), or other processing and logic elements.

Relative to methods in use at the time of filing the present application, the methods and systems disclosed herein provide a number of advantages.

Some methods and computational systems disclosed herein cluster contigs in a manner independent of the number of chromosomes for the organism. A more conservative threshold on contig-contig links for single-link clustering is applied to assemble the resulting smaller contig clusters into scaffolds, with subsequent scaffolding joining possible by various methods disclosed herein.

In some embodiments, the methods disclosed herein does not essentially involve clustering but goes straight to the spanning tree step, followed by topological tree pruning. In some embodiments, more than one clustering methods can be used, e.g. Markov Cluster Algorithm (MCL algorithm). Without being limited by theory, misassemblies can be prevented by topological pruning by treating these edges with extra care and avoiding assembly misjoins.

After fixing the order of contigs in a scaffold, the orientations can be optimized by using a dynamic programming algorithm. Such approach only read pairs mapping to pairs of contigs adjacent in the ordering contribute to the score being optimized, leaving out any contigs shorter than the maximum separation of good fragment pairs out and unassembled. To improve the orientation step, in addition to nearest-neighbor contig score interactions, contigs that are not nearest-neighbor contig score interactions can be considered by using an algorithm that incorporates data from all pairs mapping to pairs of contigs within at most w−2 intervening contigs, for example, using values of two or greater contigs in the ordering, such as 2, 3, 4, 5, 6, 7, 8, 9, 10 or more than ten.

In some embodiments, the accuracy of intercalation step can be improved. Without being bound by any theory, in assemblies with contigs shorter than the maximum separation between good read pairs after the creation of the trunk, data from contigs within a neighborhood of w contigs along the ordering are included when excluding contigs from the trunk and reinserting into it at sites that maximize the amount of linkage between adjacent contigs.

In some other embodiments, the orientation step can be improved by considering more than nearest-neighbor contig score interactions. After fixing the order of contigs in a scaffold, contig orientations are optimized by using a dynamic programming algorithm. Only read pairs mapping to pairs of contigs adjacent in the ordering contribute to the score being optimized. In some cases, an algorithm that incorporates data from all pairs mapping to pairs of contigs within at most w−2 intervening contigs in the ordering can be used for assemblies with any contigs shorter than the maximum separation of good fragment pairs. For example, using values of two or greater contigs in the ordering, such as 2, 3, 4, 5, 6, 7, 8, 9, 10 or more than ten.

In some embodiments, one can improve both ordering and orientation accuracy by integrating the ordering and orientation steps even more tightly. An initial graph can be constructed such that in this graph, nodes are contig ends, and the two end-nodes of each contig are joined by an edge. The log-likelihood ratio scores of the inter-contig edges under an assumption of a specific short gap size, was computed and followed by sorting. Working down the list in decreasing order of edge score, new edges are either accepted or rejected according to whether they would increase or decrease the total score of the assembly. It is noted that even edges with a positive score could decrease the sum of scores of contigs in the assembly because accepting an edge which implies intercalation of a contig or contigs into the gap of an existing scaffold will increase the gap sizes between pairs of linked contigs on either side of the gap, which will potentially give them a lower score.

In addition, one can efficiently compute maximum likelihood gap sizes. Overall accuracy of the reported assembly can be increased by estimating the length of unknown sequences between consecutive contigs. Given a model of the library creation process that includes a model probability density function (PDF) for the separation d between library read pairs, the maximum likelihood gap length can be found by maximizing the joint likelihoods of the separations dᵢ of the pairs spanning the gap. For differentiable model PDF, the efficient iterative optimization methods (e.g. Newton-Raphson) can be used.

An element of the methods and compositions disclosed herein is that contigs are assembled into configurations that are local optima among, for example, contig windows of 2, 3, 4, 5, 6, or more than 6 contigs for contig order, orientation or order and orientation, while being executable or obtainable in a relatively short amount of time, such as 8, 7, 6, 5, 4, 3, 2, or less than 2 hours. Thus, in some cases the methods herein allow a high degree of computational power to be brought to a computationally intensive problem without the use of a large amount of computing time and without the need to explore a globally very large computational space. Rather, local ordering achieves a modestly accurate ordering of contigs, and then computational intensity is spent optimizing local windows of contigs rather than globally optimizing all contigs at once in most cases. In some cases, using window sizes that range from 3, 4, 5, or 6, configuration optimization is done in 8, 7, 6, 5, 4, 3, 2, or less than 2 hours. For larger window sizes, configuration optimization is accomplished in a few days up to a week.

### Digital Processing Device

In some embodiments, the contig assembly methods described herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). Optionally, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

Some digital processing devices include a display to send visual information to a user, such as a cathode ray tube (CRT), a liquid crystal display (LCD), a thin film transistor liquid crystal display (TFT-LCD), an organic light emitting diode (OLED) display such as a passive-matrix OLED (PMOLED) or active-matrix OLED (AMOLED) display. Plasma displays, video projectors, or combinations of devices such as those disclosed herein.

Often, the digital processing device includes an input device to receive information from a user, such as a keyboard, a pointing device including, by way of non-limiting examples, a mouse, trackball, track pad, joystick, game controller, or stylus. In some embodiments, the input device is a touch screen or a multi-touch screen, a microphone to capture voice or other sound input or a video camera or other sensor to capture motion or visual input. In further embodiments, the input device is a Kinect, Leap Motion, or the like. Often, the input device is a combination of devices such as those disclosed herein.

### Non-Transitory Computer Readable Storage Medium

In some embodiments, the contig assembly methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the contig assembly methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program implementing the contig assembly methods includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program implementing the contig assembly methods disclosed herein includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program implementing the contig assembly methods disclosed herein includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the contig assembly methods include a web browser plug-in. In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the contig assembly methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the contig assembly methods disclosed herein include one or more databases, or use of the same. In view of the disclosure provided herein, those of skill in the art will recognize that many databases are suitable for storage and retrieval of contig information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### Diagnostic Applications

Systems and methods herein are applicable to the selection or evaluation of a drug or other therapeutic regimen. Through practice of the disclosure herein, a tissue such as a cancer tissue is evaluated as to structural rearrangements that indicate a drug candidate. For example, a local density variation or local density variation pattern is in some cases indicative of a change to a particular gene or genes. For example, a rearrangement implicated in an analysis may involve a gene truncation, deletion, or fusion, so as to form a genomic background known or suspected to be responsive to a particular therapy. An analysis is performed indicative of a therapeutic strategy, and a drug is indicated. Often, the drug or other therapeutic regimen is proposed to a medical professional or patient, or applied to the patient so as to address a medical condition related to the analyzed sample.

Alternately or in combination, systems and methods as disclosed herein are employed to monitor the success of a drug or other treatment regimen applied to an individual, such as an individual for whom a genomic rearrangement is implicated in a disorder under treatment. A sample is taken and analyzed as disclosed herein so as to identify a local density pattern. Often, but not necessarily, a local density variation is implicated in a particular genomic rearrangement associated with a disease, suggestive of a treatment approach, or indicative of disease progression (such as through abundance of the rearrangement in a sample). A treatment regimen such a s a drug treatment, alone or in combination with other treatment steps, or other steps not involving a drug, are undertaken so as to treat or ameliorate the symptoms of a condition. A second sample is taken and analyzed as disclosed herein so as to identify a local density pattern. This pattern, or resulting analysis, is compared to that observed prior to or earlier in a treatment regimen so as to assess the efficacy of the regimen, such as efficacy of a drug in reducing the abundance of a particular rearrangement in a tumor, or the efficacy of a surgical intervention or other treatment regimen in excising or reducing tissue suspected of being causative or otherwise relevant to a particular tissue disease such as a cancer tumor. Assessment variously comprises ceasing the treatment regimen, decreasing the treatment regimen, initiating a second treatment regimen, continuing the treatment regimen unchanged, increasing the treatment regimen, replacing the treatment regimen with monitoring, or other regimen input.

### Numbered Embodiments Relating to the Disclosure

The disclosure is further clarified through reference to the following numbered embodiments, which are presented in numerical order but which are understood to be readily interrelated to one another and to the remainder of the specification in addition to the interrelationships indicated by the numbers below. Numbered embodiments are presented both to further clarify the disclosure herein and to support claims reciting the subject matter of the embodiments. 1. A method of nucleic acid structural variant detection comprising a) mapping read pair information onto a reference nucleic acid scaffold; b) assigning a read pair position to a first bin such that the read pair midpoint falls within a first bin nucleic acid position range and the read pair separation falls within a first bin separation range; and c) estimating copy number variation based on a mappability value of the first bin. 2. The method of embodiment 1, further comprising normalizing the copy number variation. 3. The method of embodiment 1, further comprising visualizing mappability by plotting the mapped read density of two samples against each other. 4. A method of nucleic acid structural variant detection comprising a) mapping read pair information onto a reference nucleic acid scaffold; b) assigning a read pair position to a first bin such that the read pair midpoint falls within a first bin nucleic acid position range and the read pair separation falls within a first bin separation range; c) generating a two-dimensional image of the read pair information; wherein each pixel represents a bin; d) calculating a z-score for at least one group of four pixels sharing a common corner in the image; wherein the z-score is represented by a contrast between adjacent pixels; and e) identifying candidate hits when a z-score exceeds a threshold value. 5. The method of any one of embodiments 1-4, wherein the reference nucleic acid scaffold is a genome. 6. The method of any one of embodiments 1-4, wherein each data set is obtained from a different paired-end read direction. 7. The method of any one of embodiments 1-4, wherein the candidate hit is a translocation. 8. The method of any one of embodiments 1-4, wherein the candidate hit is an inversion. 9. The method of any one of embodiments 1-4, wherein the candidate hit is a deletion. 10. The method of any one of embodiments 1-4, wherein the candidate hit is a duplication. 11. The method of any one of embodiments 1-4, wherein the candidate hit is an interchromosomal structural variation. 12. A system for modeling a mixture of allelic variations in a sample comprising: a set of weighted genome scaffold models, wherein each genome scaffold model comprises a set of weighted chromosomes, wherein each chromosome is a linear graph of bins in the genome scaffold; and a module for calculating a log likelihood ratio of at least two genome scaffold models to predict whether a read pair sampled by a library will fall into a bin. 13. The system of any one of embodiments 1-12, further comprising at least one feature detector module, wherein the at least one feature detector module proposes candidate modifications to the genome scaffold model. 14. The system of any one of embodiments 1-13, wherein the at least one feature detector module determines the bin boundaries of a sequence variant. 15. The system of any one of embodiments 1-14, wherein the sequence variant is a translocation. 16. The system of any one of embodiments 1-14, wherein the sequence variant is an inversion. 17. The system of any one of embodiments 1-14, wherein the sequence variant is a deletion. 18. The system of any one of embodiments 1-14, wherein the sequence variant is a duplication. 19. The system of any one of embodiments 1-12, further comprising a module that generates alternative models based on input from the at least one feature detector module. 20. A method for modeling allelic variations in a sample comprising: a) generating a set of weighted genome scaffold models, wherein each genome scaffold model comprises a set of weighted chromosomes, wherein each chromosome is a linear graph of bins in the genome scaffold; b) calculating a score based on the ability of the models to describe read pair sequencing information mapped on a reference sequence, wherein a higher score value indicates a more predictive model; and c) iteratively adding additional models to maximize the score value. 21. The method of any one of embodiments 1-20, wherein the read pair sequencing information comprises an inversion. 22. The method of any one of embodiments 1-20, wherein the read pair sequencing information comprises a translocation. 23. The method of any one of embodiments 1-20, wherein the read pair sequencing information comprises a duplication. 24. The method of any one of embodiments 1-20, wherein the read pair sequencing information comprises a deletions. 25. The method of any one of embodiments 1-21, further comprising detecting features, wherein detecting features comprises joining or separating bins in the model to increase the score value. 26. The method of any one of embodiments 1-20, wherein the sample is a cancer cell. 27. A method of nucleic acid structural variant detection comprising a) mapping read pair information onto a predicted nucleic acid scaffold; b) assigning a read pair position to a first bin such that the read pair midpoint falls within a first bin nucleic acid position range and the read pair separation falls within a first bin separation range; c) generating a two-dimensional image of the read pair information; wherein each pixel represents a bin; and d) identifying at least one feature in the two-dimensional image corresponding to two sequence fragments connected by a common linking sequence fragment. 28. The method of any one of embodiments 1-27, comprising assembling the two sequence fragments connected by a common linking sequence fragment in the correct order 29. The method of any one of embodiments 1-27, wherein the method comprises discarding features corresponding to false positives. 30. A method comprising: mapping read pair sequence information onto a sequence scaffold; and identifying a local variation in density of a plurality of read pair symbols so mapped. 31. The method of any one of embodiments 1-30, comprising assigning the local variation in density to a corresponding structural arrangement feature. 32. The method of any one of embodiments 1-30, comprising restructuring the sequence scaffold so that the local variation in density is reduced. 33. The method of any one of embodiments 1-30, wherein mapping read pair sequence information onto a sequence scaffold comprises positioning a symbol indicative of a read pair such that distance of the symbol from an axis representative of the sequence scaffold indicates distance from a mapped position of a first read of a read pair on the sequence scaffold to a mapped position of a second read of the read pair on the sequence scaffold, and such that position of the symbol relative to the axis representative of the sequence scaffold indicates an average of the mapped position of the first read of the read pair and the mapped position of the second read of the read pair 34. The method of any one of embodiments 1-31, wherein restructuring the sequence scaffold comprises reordering at least some contigs of the sequence scaffold. 35. The method of any one of embodiments 1-31, wherein restructuring the sequence scaffold comprises reorienting at least one contig of the sequence scaffold. 36. The method of any one of embodiments 1-31, wherein restructuring the sequence scaffold comprises introducing a break into at least one contig of the sequence scaffold. 37. The method of any one of embodiments 1-36, further comprising introducing a sequence present at one edge of the break onto a second edge of the break. 38. The method of any one of embodiments 1-30, wherein restructuring the sequence scaffold comprises translocating a segment of a first contig into an internal region of a second contig. 39. The method of any one of embodiments 1-30, wherein mapping read pair sequence information onto a sequence scaffold comprises assigning read pair information to a plurality of bins. 40. The method of any one of embodiments 1-30, wherein identifying a local variation in density comprises identifying a region having a locally low density of symbols. 41. The method of any one of embodiments 1-30, wherein identifying a local variation in density comprises identifying a region having a locally high density of symbols. 42. The method of any one of embodiments 1-30, wherein identifying a local variation in density comprises identifying a density at a first position and a density at a second position, wherein the density at the first position and the density at the second position differ significantly. 43. The method of any one of embodiments 1-42, wherein the first position and the second position are adjacent. 44. The method of any one of embodiments 1-42, wherein the first position and the second position are equidistant from the sequence scaffold. 45. The method of any one of embodiments 1-30, wherein identifying a local variation in density comprises obtaining an expected density at a first position and an observed density at the first position. 46. The method of any one of embodiments 1-45, wherein the expected density at the first position is a density predicted by density gradient that decreases monotonically with increased distance from the axis representative of the sequence scaffold. 47. The method of any one of embodiments 1-30, wherein a local density variation of a fraction of a whole number value equal to a ploidy of a sample indicates an event in that proportion of a sample ploidy complement. 48. The method of any one of embodiments 1-30, wherein the scaffold represents a cancer cell genome. 49. The method of any one of embodiments 1-30, wherein the scaffold represents a transgenic cell genome. 50. The method of any one of embodiments 1-30, wherein the scaffold represents a gene-edited genome. 51. The method of any one of embodiments 1-32, wherein the scaffold has an N50 of at least 20% greater following the restructuring. 52. A method comprising obtaining a scaffold comprising sequence scaffold information; obtaining paired read information; deploying the paired read information such that at least some read pair information is depicted so as to indicate position of each read in a read pair relative to the scaffold and to indicate distance of one read to another as mapped on the scaffold; and identifying a local variation in density of the paired read information as deployed. 53. The method of any one of embodiments 1-52, comprising assigning the local variation in density to a corresponding structural arrangement feature. 54. The method of any one of embodiments 1-52, comprising reconfiguring the scaffold so as to decrease the local variation. 55. The method of any one of embodiments 1-52, wherein obtaining a scaffold comprising sequence scaffold information comprises sequencing a nucleic acid sample. 56. The method of any one of embodiments 1-52, wherein obtaining a scaffold comprising sequence scaffold information comprises receiving digital information representative of a nucleic acid sample. 57. The method of any one of embodiments 1-52, comprising obtaining a predicted density distribution for deployed read pair information. 58. The method of any one of embodiments 1-57, wherein the identifying comprises identifying a significant difference between the predicted density distribution and the depicted read pair information density. 59. The method of any one of embodiments 1-52, wherein identifying a local variation comprises identifying a density perturbation having a density peak at an apex of a right angle. 60. The method of any one of embodiments 1-59, wherein the apex of the right angle points to an axis representative of the scaffold. 61. The method of any one of embodiments 1-52, wherein obtaining paired end read information comprises crosslinking unextracted nucleic acids. 62. The method of any one of embodiments 1-52, wherein obtaining paired end read information comprises crosslinking nucleic acids bound in chromatin. 63. The method of any one of embodiments 1-62, wherein the chromatin is native chromatin. 64. The method of any one of embodiments 1-52, wherein obtaining paired end read information comprises binding a nucleic acid to a nucleic acid binding moiety. 65. The method of any one of embodiments 1-52, wherein obtaining paired end read information comprises generating reconstituted chromatin. 66. The method of any one of embodiments 1-52, wherein deploying the paired read information comprises assigning read pair information to a plurality of bins. 67. The method of any one of embodiments 1-52, wherein restructuring the sequence scaffold comprises reordering at least some contigs of the sequence scaffold. 68. The method of any one of embodiments 1-54, wherein restructuring the sequence scaffold comprises reorienting at least one contig of the sequence scaffold. 69. The method of any one of embodiments 1-54, wherein restructuring the sequence scaffold comprises introducing a break into at least one contig of the sequence scaffold. 70. The method of any one of embodiments 1-69, further comprising introducing a sequence at one edge of the break onto a second edge of the break. 71. The method of any one of embodiments 1-54, wherein restructuring the sequence scaffold comprises translocating a segment of a first contig into an internal region of a second contig. 72. The method of any one of embodiments 1-52, wherein the scaffold represents a cancer cell genome. 73. The method of any one of embodiments 1-52, wherein the scaffold represents a transgenic cell genome. 74. The method of any one of embodiments 1-52, wherein the scaffold represents a gene-edited genome. 75. The method of any one of embodiments 1-52, wherein the scaffold has an N50 of at least 20% greater following the restructuring. 76. The method of any one of embodiments 1-52, wherein a local density variation of a fraction of a whole number value equal to a ploidy of a sample indicates an event in that proportion of a sample ploidy complement. 77. A method of identifying a structural rearrangement in a sample relative to a sequence scaffold, comprising mapping read pair sequence information onto a sequence scaffold; identifying local density variation having a right angle edge pointing to an axis corresponding to the sequence scaffold and having bilateral symmetry along a line that bisects the right angle edge; and categorizing the sample as having a simple translocation relative to the sequence scaffold comprising segments of lengths from a translocation point at least as long as the longest furthest mapped read of the local density variation. 78. A method of identifying a structural rearrangement in a sample, comprising mapping read pair sequence information onto a sequence scaffold; identifying local density variation having a right angle edge pointing to an axis corresponding to the sequence scaffold; identifying a sub-region of the local density variation that disrupts bilateral symmetry along a line that bisects the right angle edge; and categorizing the sample as having a translocation relative to the sequence scaffold comprising a segment that lacks sequence to which a population of symmetry-restoring read pairs would map. 79. A method of identifying a structural rearrangement in a sample relative to a sequence scaffold, comprising mapping read pair sequence information onto a sequence scaffold; identifying local density variation having a right angle edge pointing to an axis corresponding to the sequence scaffold; obtaining an expected read pair density distribution curve; and identifying scaffold segments to which read pairs comprising the local density variation map; repositioning the scaffold segments such that the read pairs comprising the local density variation map to a region indicated by the expected read pair density distribution curve to have a density of the local density variation. 80. A computer monitor configured to display results of the method of any one of embodiments 1-79. 81. A computer system configured to perform computational steps of the method of any one of embodiments 1-79. 82. A visual representation of mapped read pair data of any one of embodiments 1-79. 83. A method of nucleic acid structural variant detection comprising mapping read pair information onto a predicted nucleic acid scaffold; obtaining a structural variant hypothesis; calculating a likelihood parameter that the structural variant hypothesis is consistent with the read pair information; and categorizing the nucleic acid sample as having the structural variant hypothesis if the likelihood parameter for the hypothesis is greater than a second likelihood parameter for a second hypothesis, wherein mapping read pair information onto a predicted nucleic acid scaffold comprises assigning a read pair a read pair position such that the read pair is assigned to its midpoint on the predicted nucleic acid scaffold on one axis; and such that the read pair is assigned a value corresponding to its read pair separation on a second axis 84. The method of any one of embodiments 1-83, wherein said read pair comprises a first segment mapping to a first region of a nucleic acid molecule and a second segment mapping to a second region of the nucleic acid molecule, said first segment and said second segment being nonadjacent and sharing a common phase. 85. The method of any one of embodiments 1-83, wherein a read pair position is assigned to a first bin if the read pair midpoint falls within a first bin nucleic acid position range and the read pair separation falls within a first bin separation range. 86. The method of any one of embodiments 1-85, wherein the first bin nucleic acid position range is a regular interval of the predicted nucleic acid scaffold. 87. The method of any one of embodiments 1-85 wherein the first bin separation range is a logarithmic interval of a full separation range for the read pair information. 88. The method of any one of embodiments 1-85, wherein the first bin nucleic acid range is a regular interval of a nucleic acid scaffold, and wherein first bin separation range is a logarithmic interval of a full separation range for the read pair information. 89. The method of any one of embodiments 85-88, wherein a read pair position is assigned to a second bin if the read pair midpoint falls within a second bin nucleic acid position range and the read pair separation falls within a second bin separation range. 90. The method of any one of embodiments 1-89, wherein substantially all read information is binned. 91. The method of any one of embodiments 85-90, wherein calculating the likelihood parameter comprises determining a likelihood contribution for the first bin. 92. The method of any one of embodiments 1-91, wherein the likelihood contribution for the first bin comprises a first likelihood factor proportional to a count of the read pairs mapping to the first bin. 93. The method of any one of embodiments 1-91, wherein the likelihood contribution for the first bin comprises a second likelihood factor proportional to the area of the first bin. 94. The method of any one of embodiments 1-91, wherein the likelihood contribution for the first bin comprises a first likelihood factor proportional to a count of the read pairs mapping to the first bin, and wherein the likelihood contribution for the first bin comprises a second likelihood factor proportional to the area of the first bin. 95. The method of any one of embodiments 1-94, comprising determining a likelihood contribution for a second bin that does not overlap in area with the first bin. 96. The method of any one of embodiments 1-95, wherein the likelihood parameter comprises the likelihood contribution of the first bin and the likelihood contribution of the second bin. 97. The method of any one of embodiments 1-96, wherein the likelihood parameter comprises the likelihood contribution of a third bin. 98. The method of any one of embodiments 1-97, wherein the likelihood parameter comprises a likelihood contribution for substantially all binned read pair information. 99. The method of any one of embodiments 78-98, wherein the hypothesis comprises a structural variation having a left edge and a length. 100. The method of any one of embodiments 1-99, wherein the structural variation has an orientation that is at least one of a deletion, an inversion, a direct duplication, an outward inverted duplication, and an inward inverted duplication. 101. The method of any one of embodiments 99-100, wherein the second hypothesis comprises a structural variant differing in at least one of a left edge, a length and a structural orientation. 102. The method of any one of embodiments 1-101, wherein said nucleic acid structural variant is homozygous in said nucleic acid sample. 103. The method of any one of embodiments 78-101, wherein said nucleic acid structural variant is heterozygous in said nucleic acid sample. 104. A method of visualizing a putative structural variation in a nucleic acid sample, comprising the steps of assigning a population of sequence reads to a population of numbered bins, and assigning a likelihood parameter of a read comprising a structural variation edge falling within a first bin of said population of bins, wherein said likelihood parameter for said first bin comprises a first likelihood component that includes the number of reads mapping to the first bin and a second component that includes the area of the first bin. 105. The method of any one of embodiments 1-104, comprising plotting the likelihood of structural variation as a function of bin number. 106. The method of any one of embodiments 1-104, wherein said likelihood parameter for said first bin comprises a convolution of a first likelihood component that includes a number of reads mapping to the first bin and a second component that includes an area of the first bin. 107. The method of any one of embodiments 1-106, wherein said likelihood parameter comprises a likelihood component relating a structural variant prediction to the number of reads mapping to the first bin and a likelihood component that includes the area of the first bin. 108. The method of any one of embodiments 1-104, wherein said bin population shares a common bin width spanning a fixed nucleic acid distance. 109. The method of any one of embodiments 1-104, wherein said bin population varies as to bin height among its members. 110. The method of any one of embodiments 1-109, wherein bin height appears constant when plotted on a logarithmic axis. 111. The method of any one of embodiments 1-104, wherein the likelihood parameter relates to a probability of a sequence read, comprising a junction of a structural variation having a left edge and a length, mapping to said first bin. 112. The method of any one of embodiments 1-111, wherein the structural variation has an orientation that is at least one of a deletion, an inversion, a direct duplication, an outward inverted duplication, and an inward inverted duplication. 113. The method of any one of embodiments 1-104, wherein said sequence reads comprise read pairs. 114. The method of any one of embodiments 1-113, wherein a read pair comprises a first segment mapping to a first region of a nucleic acid molecule and a second segment mapping to a second region of the nucleic acid molecule, said first segment and said second segment being nonadjacent and sharing a common phase. 115. A method of identifying a structural variant in a nucleic acid sample comprising the steps of obtaining mapped read pair data for the nucleic acid sample; obtaining a nucleic acid scaffold sequence; obtaining likelihood probability information for each of a plurality of structural variant hypotheses comparing the read pair data to the nucleic acid scaffold sequence; and identifying a most probable hypothesis among the structural variant hypotheses; wherein said method evaluates at least 10 Mb of nucleic acid scaffold sequence per minute. 116. The method any one of embodiments 1-115 comprising mapping read pair information onto the nucleic acid scaffold sequence; obtaining a structural variant hypothesis; calculating a likelihood parameter that the structural variant hypothesis is consistent with the read pair information; and categorizing the nucleic acid sample as having the structural variant hypothesis if the likelihood parameter for the hypothesis is greater than a second likelihood parameter for a second hypothesis. 117. The method of any one of embodiments 1-116, wherein mapping read pair information onto the nucleic acid scaffold sequence comprises assigning a read pair a read pair position such that the read pair is assigned to its midpoint on the predicted nucleic acid scaffold on one axis; and the read pair is assigned a value corresponding to its read pair separation on a second axis 118. The method of any one of embodiments 116-112, wherein said read pair comprises a first segment mapping to a first region of a nucleic acid molecule and a second segment mapping to a second region of the nucleic acid molecule, said first segment and said second segment being nonadjacent and sharing a common phase. 119. The method of any one of embodiments 1-117, wherein a read pair position is assigned to a first bin if the read pair midpoint falls within a first bin nucleic acid position range and the read pair separation falls within a first bin separation range. 120. The method of any one of embodiments 1-119, wherein the first bin nucleic acid position range is a regular interval of a nucleic acid scaffold. 121. The method of any one of embodiments 1-119, wherein the first bin separation range is a logarithmic interval of a full separation range for the read pair information. 122. The method of any one of embodiments 1-119, wherein the first bin nucleic acid position range is a regular interval of a nucleic acid scaffold, and wherein first bin separation range is a logarithmic interval of a full separation range for the read pair information. 123. The method of any one of embodiments 119-122, wherein a read pair position is assigned to a second bin if the read pair midpoint falls within a second bin nucleic acid position range and the read pair separation falls within a second bin separation range. 124. The method of any one of embodiments 1-123, wherein substantially all read information is binned. 125. The method of any one of embodiments 119-119, wherein calculating the likelihood parameter comprises determining a likelihood contribution for the first bin. 126. The method of any one of embodiments 1-125, wherein the likelihood contribution for the first bin comprises a first likelihood factor proportional to a count of the read pairs mapping to the first bin. 127. The method of any one of embodiments 1-120, wherein the likelihood contribution for the first bin comprises a second likelihood factor proportional to the area of the first bin. 128. The method of any one of embodiments 1-120, wherein the likelihood contribution for the first bin comprises a first likelihood factor proportional to a count of the read pairs mapping to the first bin, and wherein the likelihood contribution for the first bin comprises a second likelihood factor proportional to the area of the first bin. 129. The method of any one of embodiments 1-123, comprising determining a likelihood contribution for a second bin that does not overlap in area with the first bin. 130. The method of any one of embodiments 1-124, wherein the likelihood parameter comprises the likelihood contribution of the first bin and the likelihood contribution of the second bin. 131. The method of any one of embodiments 1-130, wherein the likelihood parameter comprises the likelihood contribution of a third bin. 132. The method of any one of embodiments 1-126, wherein the likelihood parameter comprises a likelihood contribution for substantially all binned read pair information. 133. The method of any one of embodiments 115-127, wherein the hypothesis comprises a structural variation having a left edge and a length. 134. The method of any one of embodiments 1-128, wherein the structural variation has an orientation that is at least one of a deletion, an inversion, a direct duplication, an outward inverted duplication, and an inward inverted duplication. 135. The method of any one of embodiments 134-129, wherein the second hypothesis comprises a structural variant differing in at least one of a left edge, a length and a structural orientation. 136. The method of any one of embodiments 111-130, wherein said nucleic acid structural variant is homozygous in said nucleic acid sample. 137. The method of any one of embodiments 111-130, wherein said nucleic acid structural variant is heterozygous in said nucleic acid sample. 138. A method of selecting a treatment regimen, comprising performing the method of any one of the preceding embodiments, identifying a rearrangement, and identifying a treatment regimen consistent with the rearrangement. 139. The method of any one of embodiments 1-133, wherein the treatment regimen comprises drug administration. 140. The method of any one of embodiments 1-133, wherein the treatment regimen comprises tissue excision. 141. A method of evaluating a treatment regimen, comprising performing the method of any one of the preceding embodiments a first time, administering the treatment regimen, and performing the treatment regimen a second time. 142. The method of any one of embodiments 1-136, comprising discontinuing the treatment regimen. 143. The method of any one of embodiments 1-136, comprising increasing dosage of the treatment regimen. 144. The method of any one of embodiments 1-136, comprising decreasing dosage of the treatment regimen. 145. The method of any one of embodiments 1-136, comprising continuing the treatment regimen. 146. The method of any one of embodiments 136-140, wherein the treatment regimen comprises a drug. 147. The method of any one of embodiments 136-140, wherein the treatment regimen comprises a surgical intervention.

### DISCUSSION OF THE ACCOMPANYING FIGURES

At FIG. 1 one sees an exemplary workflow of 8 steps for methods used to process paired-end read data. Exemplary steps include read mapping (mapping paired sequence reads from one individual against a reference), read binning (group reads by one or more properties), copy number estimation (copy number variation, CNV), normalization, de novo feature detection, breakpoint refinement, candidate scoring, and reporting. In some instances, steps are repeated or skipped entirely during the analysis of paired-end read data.

At FIGS. 2A-2C one sees pairs of plots, each plot with bins corresponding to a range of midpoint positions of a mapped read pair on the x axis, with a scale from 0 to 12000 bases in 20,000 bp increments, and the estimated copy number on the Y axis as a logarithmic scale between 0.1 and 10. For the reference samples CT407 (top) in FIG. 2A, CT418 (top) in FIG. 2B, and CT416 in FIG. 2C, most of the bases are present as a single copy, represented by an area of high plot density in the center of the vertical axis. The samples represented by the bottom plots CT410 in FIG. 2A and CT417 in FIG. 2B show significant deviation from 1, with bins having more or fewer than one copy number. For example, sample CT410 has an increase in copy number for bins at approximately 10,000 to 10,500 bases. FIG. 2D shows a two-dimensional scatterplot with copy numbers for sample CT410 on the x-axis, and CT407 on the y-axis with each point representing the copy number for a corresponding bin in each sample. The majority of points are concentrated at coordinates (1,1) on y=x diagonal line which corresponds to a single copy at that bin in both samples. Points not falling near the diagonal line represent a significant difference in copy number between the two samples. For example, points corresponding to (100, 10) represent bins that have a 10 fold increase in copy number of CT410 compared to CT407.

At FIG. 3A one sees a plot of midpoint positions of mapped read pairs on the x axis, with a scale of 5.31×10⁷ to 5.36×10⁷ base pairs in 0.01×10⁷ increments, and the read pair separation plotted on they axis with a scale of 0 to 200,000 bases (20,000 base increments) for chromosome 7 of sample NA12878. This plot does not show any clear structural variations, as evidenced by most of the points falling near 0 on the y axis. This suggests that most of the read pairs correspond to adjacent segments on the scaffold. At FIG. 3B and FIG. 3C, showing an x axis scale of 5.41×10⁷ to 5.46×10⁷ and a y axis scale of 0 to 200,000 (20,000 base increments) and 100 to 100,000 (log scale). In these plots, one sees an inversion present between about 5.42×10⁷ and 5.44×10⁷ bases, where there are gaps in the data. At FIG. 3D, one sees an exemplary depiction of an inversion located between locations a and b, wherein roughly half the points (grey) remain near the axis, and the other half are reflected above the midway point between location a and b. In this example, the light colored points remaining near the axis indicate a heterozygous inversion, wherein only one chromosome in a pair is inverted. In some instances, the plot is rotated 45 degrees, wherein the x axis is on a y=−x diagonal.

At FIG. 4A one sees an example of various structural variations manifested as a redistribution of mapped read pairs into areas formed by lines that are a 45 degree angle from the x axis. FIG. 4B depicts a number system for defining the density areas formed by lines that are a 45 degree angle from the axis. FIGS. 4C-4G depict exemplary methods of defining areas of density for various structural variations. In some instances the areas of density create patterns which are kernels. The patterns defined are variously used to predict density variations that are indicative of discrepancies between mapped read pair data and the scaffold. For example, FIG. 4C, FIG. 4D, FIG. 4E, FIG. 4F, and FIG. 4G define in some cases areas of local density change expected for a deletion, inversion, direct tandem duplication, inverted tandem duplication (right), or inverted tandem duplication (left), respectively. Exemplary equations for defining the predicted variation in densities for each of the regions 0-3 are shown on the left side of the respective figures.

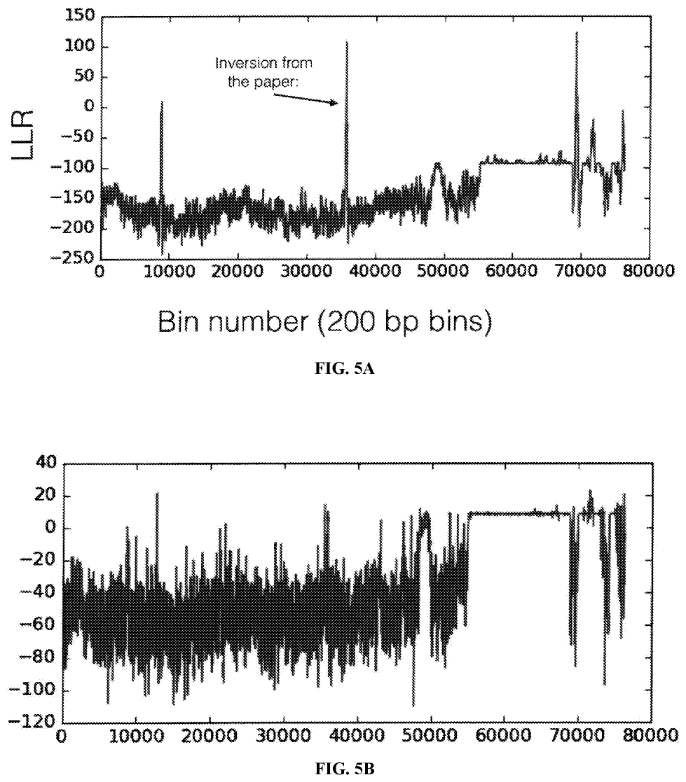

At FIG. 5A one sees a plot of predicted structural variations comprising an x axis of 200 base pair bin numbers with a scale from 0 to 80,000 in intervals of 10,000, and a y axis representing the log likelihood ratio (LLR) on a scale between −250 and 150 in intervals of 50. The log likelihood ratio in some instances represents the likelihood that a structural variation has occurred verses the likelihood that the variation has not occurred. Higher values indicate a more likely variation, for example the spike seen at about bin 36000 corresponds to a known inversion. At FIG. 5B one sees a plot of predicted structural variations comprising an x axis of 200 base pair bin numbers with a scale from 0 to 80,000 in intervals of 10,000, and a y axis representing the log likelihood ratio (LLR) on a scale between −120 and 40 in intervals of 20. In this example, the relatively negative values between bins about 55000 and 68000 indicate a 10 Kb heterozygous deletion is present. At FIG. 5C one sees a plot of predicted structural variations comprising an x axis of 200 base pair bin numbers with a scale from 0 to 80,000 in intervals of 10,000, and a y axis representing the log likelihood ratio (LLR) on a scale between −100 and 60 in intervals of 20. In this example, the relatively negative values between bins about 55000 and 68000 indicate that a 26 Kb heterozygous duplication (L) is present.

At FIG. 6A and FIG. 6B one sees exemplary read distribution patterns that in some cases depict reciprocal translocations, in this case a square, divided into four regions. In some instances, this pattern is a kernel or a feature. The read density in this case is distributed in diagonal areas formed by the intersection of two lines. At FIG. 6C one sees areas depicted as foreground (fg) and background (bg) regions, which are compared as a ratio of fg to bg to establish in some instances a z-score. The z-score often is used to identify a feature from noise. At FIG. 6D one sees a plot of read pair data mapped on a scaffold, with features identified (circled). In some cases, an area of high or low read density is not reflected across the center of the square (upper right circle), as compared to features in the lower left showing a reflection of density across the center of the square. In this example, the read pair density decreases at 45 degree angle gradient away from the center of the square, where the highest density is found. In some cases the “bowtie” structure exemplified by the two circled features in the lower left corresponds to a translocation.

At FIG. 7 one sees an image of read pairs mapped onto a scaffold, illustrating intra-chromosomal rearrangements as visualized by areas of unexpectedly high or low read density off the diagonal y=−x axis. These areas located off the diagonal axis correspond to mapped read pairs that are separated by distances longer than the read, indicating potential discrepancies in the assembly of the scaffold.

At FIG. 8A one sees an illustration of a “2^{nd} degree link” assembly situation, wherein two different assembly outcomes are possible from analyzing only first-order read pairs. The three sequences in each set above the arrow correspond to the native sequence arrangement (the scaffold): sequence a-b, c-d-e, and f-g. However, rearrangement (represented by the arrows) of fragments in the sequences results in two potential arrangements: a-d-e and c-d-g, or a-d-g, which are indistinguishable through first-order read pair analysis because both potential rearrangements will result in rearranged sequences having a read pair mapping fragment a to d, and d to g. At FIG. 8B one sees an illustration depicting read pair data mapped to a scaffold, with data on the axis not shown. Two features are identified (boxes with shading representing read pair density, with decreasing intensity along a gradient extending away from the diagonal axis at a right angle, in the box, labeled with a symbol of a smaller and larger circle touching each other). A linear arrangement of fragments a-g in alphabetical order is used as the scaffold. Read pair data from the two “off-axis” features indicates a connection between fragments a-d and d-g. Additionally, the lack of signal marked by concentric circles indicates that fragment a and g are not connected by intervening sequence d. At FIG. 8C one sees a similar graph depicting the expected pattern for an a-d-g linkage. The connectivity of a-d and d-g is illustrated by the features identified at the small and large circle symbols. Although fragments a and g are not directly connected, a shaded region is observed corresponding to read pairs that bridge intervening sequence d, and features corresponding to a-f and c-g are absent (concentric circles), further supporting the hypothesis of a-d-g connectivity. In FIG. 8D one sees a similar graph depicting the expected pattern for an a-d-g linkage, with key features visible in the shaded boxes. In some instances, the “bridging” feature corresponding to a-g indicates a false positive fusion call between fragments a and g. In other cases, features at d-g indicate a false positive fusion call wherein no additional fragments are present on the left side of fragment d in d-g. At FIG. 8E one sees a plot showing how abundance of a read pair in a mixture (g) and the gap size/distance (γ) are predictive of the expected changes in density (contours lines). For example, the left plot depicts a rapid decrease in read density (from the middle of the contour lines) when the distance between read pairs (g) is small, and abundance is low. The right plot depicts a rapid decrease in read density (from the middle of the contour lines) when the distance between read pairs (g) is large, and abundance is high. In some instances, the rate at which read density decreases is used to predict blocking edges between sequence fragments. For example, a sharp and rapid decrease in read density adjacent to one kernel indicates the lack of an adjacent kernel. Comparison of expected read density for an area in some cases is used for minimizing false positive kernel calls. Often a putative kernel will possess a read density that is higher than expected for a terminal fragment (connected to only one additional fragment), and a terminal fragment will not be identified as such. Alternatively, a putative kernel will possess a read density that is less than expected for a fusion event, and a fusion event will not be identified as such. In certain cases, a rapid decrease in density is referred to as a “step”, to be contrasted with a gradual change in density. Expected density may also be defined or described by geometrical considerations, such as symmetry. For example, a symmetrical change in read density indicates an isolated discrepancy from the scaffold model, wherein an asymmetric change in read density optionally indicates the presence of an additional, adjacent discrepancy.

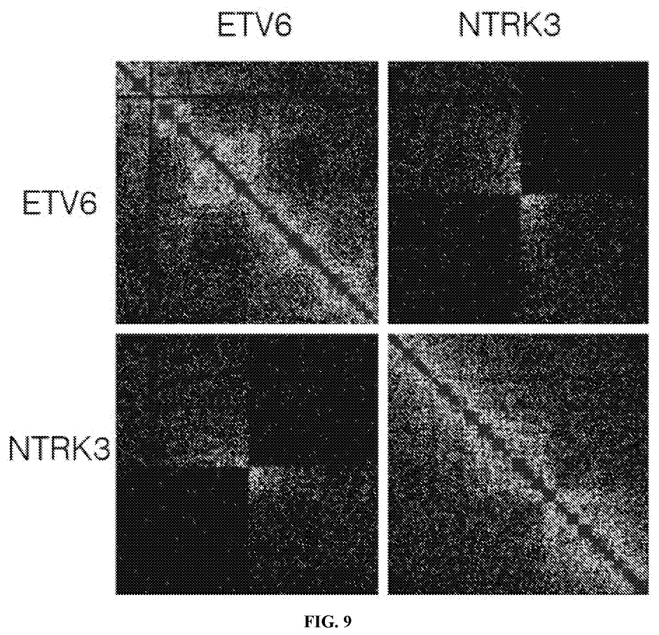

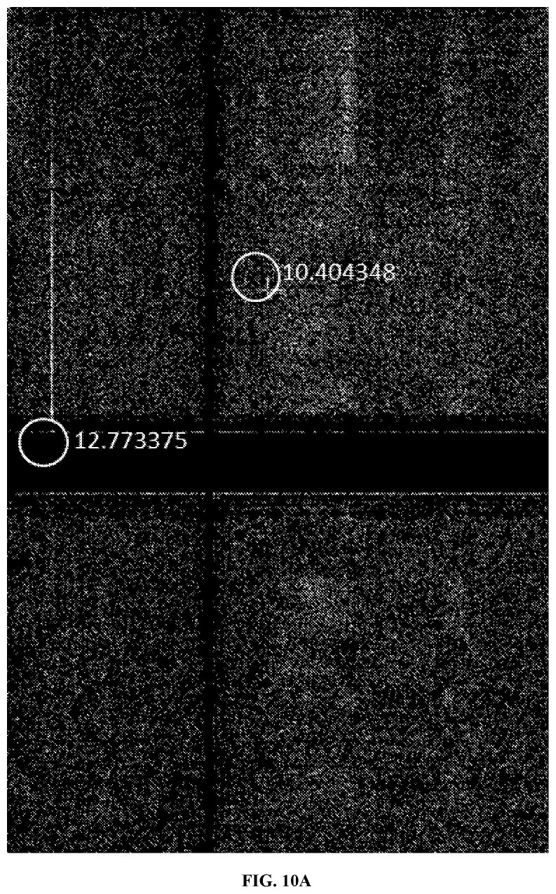

At FIG. 9 one sees an image of read pairs from two genes mapped onto a scaffold, illustrating structural variations as visualized by areas of unexpectedly high or low read density off the diagonal y=−x axis. The bowtie shaped density distributions in the upper right and lower left boxes areas indicate a reciprocal translocation between genes ETV6 and NTRK3.

At FIG. 10A-10C one sees an image analysis-based result at the same pair of chromosomes compared in three different samples. The circled regions correspond to identified features representing structural variations.

At FIG. 11A-11C one sees an image depicting median normalized read density (over 10 samples) for chromosome 1 versus chromosome 7 (FIG. 11A), chromosome 2 versus chromosome 5 (FIG. 11B), and chromosome 1 versus chromosome 1 (FIG. 11C).

At FIG. 12A and FIG. 12B one sees images depicting various bin handling approaches for mapped read pair data, which places read pairs into groups. FIG. 12A shows equal bin sizes and FIG. 12B shows bin interpolation.

At FIG. 13 one sees an image depicting a genome-wide scanning analysis pipeline, with identified features corresponding to structural variations. Sample calls made by the analytical pipeline are shown circled in white. FIG. 13 shows a plot of chromosome 3 versus chromosome 6, with 250 k bins.

At FIG. 14A one sees a graph of the probability of an insert in a particular range as a function of insert distance in base pairs (bp) for a preserved sample (e.g., an FFPE sample) analyzed by techniques of the present disclosure. At FIG. 14B one sees a similar graph for a sample analyzed using a Chicago method. In both graphs, the x-axis shows the insert distance (bp), from 0 to 300,000 (in 50,000 bp increments), while the y-axis shows the probability of an insert of that distance, from 10⁰ at the top of the axis to 10⁻⁸ at the bottom of the axis (logarithmic).

At FIG. 15A and FIG. 15B one sees graphs of mapped locations on a reference sequence, e.g., GRCh38, of read pairs generated from proximity ligation of DNA from re-assembled chromatin are plotted in the vicinity of structural differences between GM12878 and the reference. In FIG. 15A, the x axis is Read Position 1 (in Mb) with a scale of 54.2 to 54.55 in 0.05 Mb increments. They axis is Read Position 2 (in Mb) with a scale of 54.15 to 54.55 in 0.05 Mb increments. In FIG. 15B, the x axis is Read Position 1 (in Mb) with a scale of 78.85 to 79.15 in 0.05 Mb increments. They axis is Read Position 2 (in Mb) with a scale of 78.8 to 79.2 in 0.05 Mb increments. Each read pair generated is represented both above and below the diagonal. Above the diagonal, shades indicates map quality score on scale shown; below the diagonal shades indicate the inferred haplotype phase of generated read pairs based on overlap with a phased SNPs. In some embodiments, plots generated depict inversions with flanking repetitive regions, as illustrated in FIG. 15B. In some embodiments, plots generated depict data for a phased heterozygous deletion, as illustrated in FIG. 15B. Mapping paired sequence reads from one individual against a reference is the most commonly used sequence-based method for identifying differences in contiguous nucleic acid or genome structure like inversions, deletions and duplications (Tuzun et al., 2005). FIG. 15A and FIG. 15B show how read pairs generated by proximity ligation of DNA from re-assembled chromatin from GM12878 mapped to the human reference genome GRCh38 reveal two such structural differences.

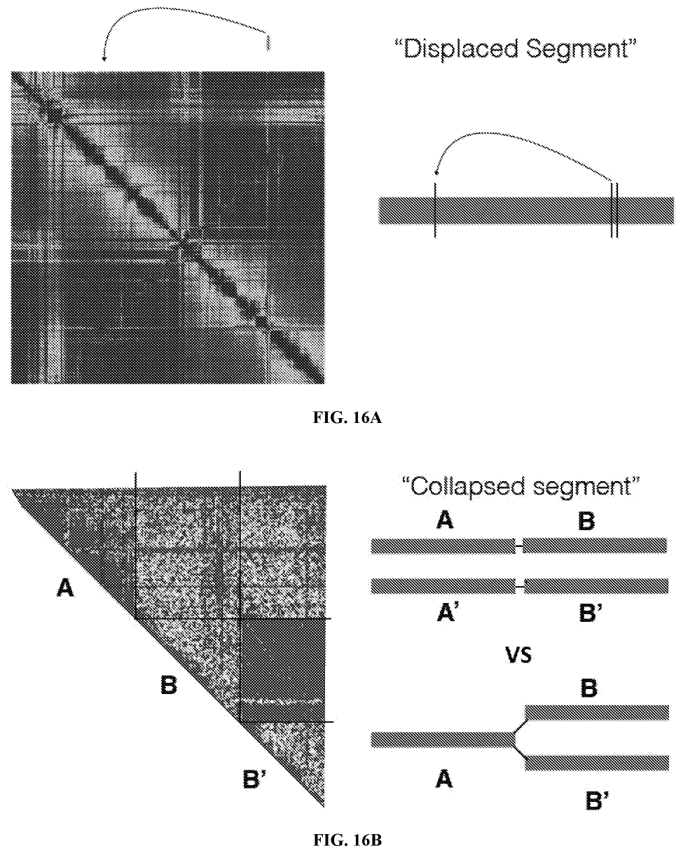

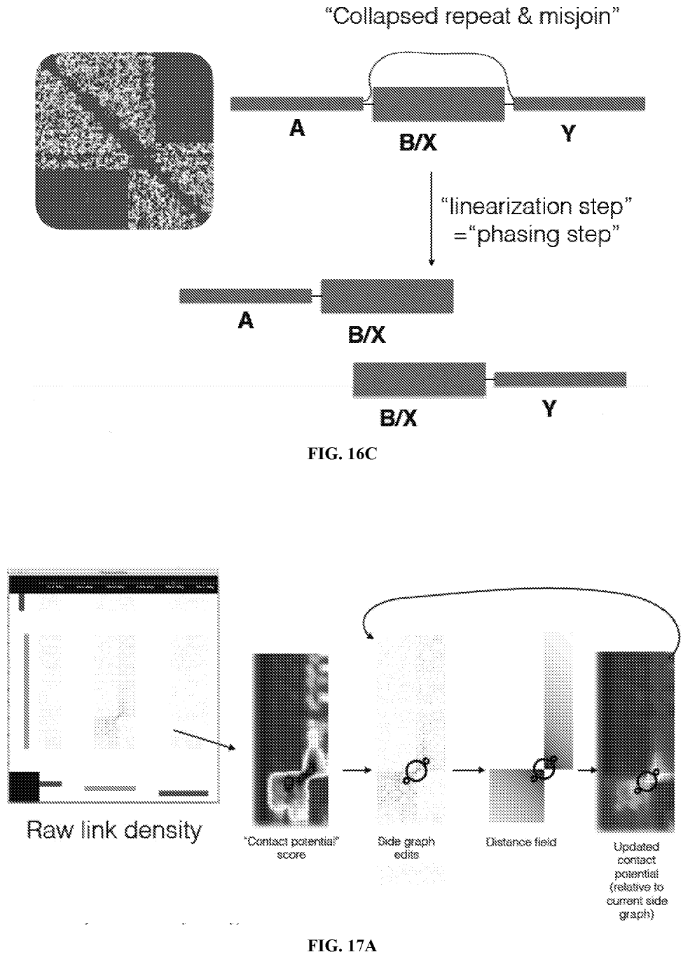

At FIG. 16A-16C one sees illustrations of exemplary sequencing disparities (right) between mapped read pair data and a reference scaffold, and images depicting these events (left). For example, in FIG. 16A, one sees a displaced segment disparity wherein a scaffold position maps to a large number of positions on a single axis (either as a thin horizontal or vertical line). The vertical line above the plot indicates the location of the displaced segment, and then arrow indicates the correct placement of this vertical band in the scaffold. Optionally, the model is updated by repositioning the fragment corresponding to the displaced segment to its correct place in the scaffold. At FIG. 16B one sees a collapsed fragment case in which fragments A and A′ are highly similar and mapped together, but fragments B and B′ are highly dissimilar (right, top), resulting in the generation of a scaffold which incorrectly orders the fragments as A-B-B′ (right, bottom). This discrepancy is identified from the off-diagonal areas of unexpected low read density in image generated by the mapped read pair (left, area above B′), and alternately or in combination by the higher than expected read density near the axis for fragment A (indicating two copies relative to B/B′). If fragments B and B′ were ordered as the scaffold suggested (adjacent), read density near the diagonal axis corresponding to this adjacency would be expected, as seen between the A-B fragment. Additionally, higher than expected density is observed in the area corresponding to A-B′, further suggesting that B and B′ are independently adjacent to A, but not each other. Optionally, the model is corrected by moving B′ to a different chromosome, duplicating A on that chromosome, and updated the copy number. At FIG. 16C one sees a collapsed repeat and misjoin case wherein two fragments A and Y are each adjacent to a highly similar sequence B/X, but A and Y are present on different chromosomes. The generated scaffold incorrectly arranged the fragments as A-(B/X)-Y, wherein B/X has been collapsed, and A-Y are improperly linked. This discrepancy is identified from mapped read pair data in the image (left), where an area of unexpectedly low read density is seen on either side of the diagonal axis, but additional lines of low density extend outward from the feature at 45 degree angles from the diagonal axis. Alternately or in combination this discrepancy is also identified by an area of higher than expected read density near the axis, corresponding to two copies of B/X relative to A or Y. Optionally, the model is corrected by breaking the connection of B/X and Y, and then duplicating B/X and attaching it to Y.

At FIG. 17A one sees an exemplary workflow for improving the quality of mapped read pair data (model optimization), including steps of obtaining raw link density data, generating a contact potential score, making side graph edits, generating a distance field, and updated the contact potential relative to the current side graph. In some cases, this process results in an interactively updated graph-based model of a genome. In some instances, this process is iterated to improve the quality of mapped read pair data for feature identification. At FIG. 17B one sees an image of raw link density read pair data mapped on to a scaffold prior to model optimization for a potato chromosome. At FIG. 17C one sees the same an image of read pair data mapped on to a scaffold after model optimization for a potato chromosome. The resulting image in some cases has fewer off-axis areas of local high and low density, indicating a better fit of the scaffold model to the read pair data.

At FIG. 18A-18D one sees examples of computer systems or networks for implemented methods described herein. For example, FIG. 18A shows an exemplary computer system that is programmed or otherwise configured to implement the methods provided herein. For example, At FIG. 18B one sees an example of a computer system that can be used in connection with example embodiments of the present invention. At FIG. 18C one sees a block diagram illustrating a first example architecture of a computer system 700 that can be used in connection with example embodiments of the present invention. At FIG. 18D one sees a diagram demonstrating a network 2100 configured to incorporate a plurality of computer systems, a plurality of cell phones and personal data assistants, and Network Attached Storage (NAS) that can be used in connection with example embodiments of the present invention. At FIG. 18E one sees a block diagram of a multiprocessor computer system 900 using a shared virtual address memory space that can be used in connection with example embodiments of the present invention. In some instances, computer systems and networks perform the methods described herein without user supervision.

### Definitions

As used herein and in the appended claims, the singular forms “a,” “and,” and “the” include plural referents unless the context clearly dictates otherwise. Thus, for example, reference to “contig” includes a plurality of such contigs and reference to “probing the physical layout of chromosomes” includes reference to one or more methods for probing the physical layout of chromosomes and equivalents thereof known to those skilled in the art, and so forth.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “sequencing read” as used herein, refers to a fragment of DNA in which the sequence has been determined.

The term “contigs” as used herein, refers to contiguous regions of DNA sequence. “Contigs” can be determined by any number methods known in the art, such as, by comparing sequencing reads for overlapping sequences, and/or by comparing sequencing reads against databases of known sequences in order to identify which sequencing reads have a high probability of being contiguous.

The term “subject” as used herein can refer to any eukaryotic or prokaryotic organism.

The term “naked DNA” as used herein can refer to DNA that is substantially free of complexed proteins. For example, it can refer to DNA complexed with less than about 50%, about 40%, about 30%, about 20%, about 10%, about 5%, or about 1% of the endogenous proteins found in the cell nucleus.

The term “reconstituted chromatin” as used herein can refer to chromatin formed by complexing nucleic acid binding moieties to a nucleic acid such as naked DNA. In some cases these moieties are nucleic acid proteins such as nuclear proteins or histones, but other moieties such as nanoparticles are also contemplated.

The term “read pair” or “read-pair” as used herein can refer to two or more elements that are linked to provide sequence information. In some cases, the number of read-pairs can refer to the number of mappable read-pairs. In other cases, the number of read-pairs can refer to the total number of generated s.

A “tissue sample” as used herein, refers to a biological sample from an individual or an environment potentially comprising nucleic acids. Tumors, for example, are considered tissues, and a sample taken from a tumor constitutes a tissue sample, but in some cases the term refers to samples taken from a heterogeneous environment such as a stomach or intestine section, or an environmental sample comprising nucleic acids from a plurality of sources spatially distributed relative to one another.

“About,” as used herein in reference to a number refers to that number +/−10% of that number. As used in reference to a range, ‘about’ refers to a range having a lower limit 10% less than the indicated lower limit of the range and an upper limit that is 10% greater than the indicated upper limit of the range.

A “probe” as used herein refers to a molecule that conveys information through binding to a target. Exemplary probes include oligonucleotide molecules and antibodies. Oligonucleotide molecules may act as probes by annealing to a target and conveying information either by changing a fluorescence characteristic, or alternately by annealing to a target and facilitating synthesis of a product such as an amplicon indicative of presence of the target. That is, the term probe as used herein variously contemplates antibody probes and other small molecule probes, as well as oligonucleic acid molecules, either acting by generating a signal directly through hybridization to a target leading to, for example, a change in fluorescence status, or acting by facilitating synthesis of an amplicon indicative of target presence.

As used herein, the phrase “at least one of” when followed by a series such as ‘A, B, C, D’ refers to a single member of the series (A or B or C or D), two members of the series, three members of the series, up to and including all of the members of series (A, B, C, and D), and in some cases also including additional unlisted members. “At least one of” a series does not necessarily imply that there is one representative of each member of the series.

As used herein, a DNA protein complex is destroyed or disrupted when proteins and nucleic acids are no longer assembled so as to form a complex. In some cases the complexes are completely denatured or disassembled, so that no protein DNA binding remains. Alternately, in some cases a DNA protein complex is substantially destroyed when a first nucleic acid segment and a second nucleic acid segment are no longer held together independent of any phosphodiester bond.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

The following examples are intended to illustrate but not limit the disclosure. While they are typical of those that might be used, other procedures known to those skilled in the art may alternatively be used.

### EXAMPLES

### Example 1

A sample comprising three chromosomes is suspected of having at least some genomic material having undergone at least one genomic rearrangement relative to a reference scaffold. The sample comprises a first chromosome having segments a and b, a second chromosome comprising segments c, d, and e, and a third chromosome comprising segments f and g.

Read pair information is obtained for the sample, and the read pairs are mapped relative to the reference scaffold.

A local density variation representing a substantial overrepresentation of read pairs mapping to segments a and d is observed. It is concluded that a rearrangement bringing a and d into physical linkage with one another has occurred.

The local density variation is analyzed in further detail. It is observed that, at the peak density for this local density variation, read pair bin occupancy, as a measurement of density, matches that of read pair density immediately off the axis. It is concluded that segments a and d are adjacent in at least one rearrangement event.

The local density variation is observed as to its symmetry. It is seen that the local density variation is substantially bilaterally symmetric along a line bisecting its right angle edge nearest to the scaffold axis with the level of resolution of the mapping. It is observed that the translocation comprises segments of both a and d that are at least as long as the level of resolution of the assay. It is concluded that the event is a simple translocation bringing a adjacent to d.

### Example 2

A sample comprising three chromosomes is suspected of having at least some genomic material having undergone at least one genomic rearrangement relative to a reference scaffold. The sample comprises a first chromosome having segments a and b, a second chromosome comprising segments c, d, and e, and a third chromosome comprising segments f and g.

Read pair information is obtained for the sample, and the read pairs are mapped relative to the reference scaffold.

A local density variation representing a substantial overrepresentation of read pairs mapping to segments a and d is observed. It is concluded that a rearrangement bringing a and d into physical linkage with one another has occurred.

The map is examined in further detail. It is observed that a and d are not involved in any other substantial off-axis local density variations. It is concluded that segments a and d are adjacent in one rearrangement event.

### Example 3

A sample comprising three chromosomes is suspected of having at least some genomic material having undergone at least one genomic rearrangement relative to a reference scaffold. The sample comprises a first chromosome having segments a and b, a second chromosome comprising segments c, d, and e, and a third chromosome comprising segments f and g.

Read pair information is obtained for the sample, and the read pairs are mapped relative to the reference scaffold.

A local density variation representing a substantial overrepresentation of read pairs mapping to segments a and d is observed. It is concluded that a rearrangement bringing a and d into physical linkage with one another has occurred.

The map is examined in further detail. It is observed that d is involved in other substantial off-axis local density variations. Segment d is observed to be involved in a local density variation having read pair complements that map to g. It is concluded that segments d and g are involved in a rearrangement event bringing them into physical linkage.

The local density variation is analyzed in further detail. It is observed that, at the peak density for this d to g local density variation, read pair bin occupancy, as a measurement of density, matches that of read pair density immediately off the axis. It is concluded that segments d and g are adjacent in at least one rearrangement event.

The map is examined in further detail. It is observed that a is involved in other substantial off-axis local density variations. Segment a is observed to be involved in a local density variation having read pair complements that map to g. It is concluded that segments d and g are involved in a rearrangement event bringing them into physical linkage.

The local density variation is analyzed in further detail. It is observed that, at the peak density for this a to g local density variation, read pair bin occupancy, as a measurement of density, is substantially lower than that of read pair density immediately off the axis. It is concluded that segments a and g are not adjacent in at least one rearrangement event.

The a-d and d-g local density variations are examined in more detail. It is observed that each lacks bilateral symmetry along a line drawn from a right angle edge closest to the axis. It is concluded that a translocation of a segment of d that is within the level of resolution of the map has occurred.

### Example 4

A sample comprising three chromosomes is suspected of having at least some genomic material having undergone at least one genomic rearrangement relative to a reference scaffold. The sample comprises a first chromosome having segments a and b, a second chromosome comprising segments c, d, and e, and a third chromosome comprising segments f and g.

Read pair information is obtained for the sample, and the read pairs are mapped relative to the reference scaffold.

A local density variation representing a substantial overrepresentation of read pairs mapping to segments a and d is observed. It is concluded that a rearrangement bringing a and d into physical linkage with one another has occurred.

The local density variation is analyzed in further detail. It is observed that, at the peak density for this a to d local density variation, read pair bin occupancy, as a measurement of density, is approximately half that of read pair density immediately off the axis. It is concluded that segments a and d are adjacent in at least one rearrangement event.

The map is examined in further detail. It is observed that d is involved in other substantial off-axis local density variations. Segment d is observed to be involved in a local density variation having read pair complements that map to g. It is concluded that segments d and g are involved in a rearrangement event bringing them into physical linkage.

The local density variation is analyzed in further detail. It is observed that, at the peak density for this d to g local density variation, read pair bin occupancy, as a measurement of density, is approximately half that of read pair density immediately off the axis. It is concluded that segments d and g are adjacent in at least one rearrangement event.

The map is examined in further detail. It is observed that a not involved in any local density variation having read pair complements that map to g. It is concluded that segments a and g are not involved in a rearrangement event bringing them into physical linkage.

The a-d and d-g local density variations are examined in more detail. It is observed that each shows bilateral symmetry along a line drawn from a right angle edge closest to the axis. It is concluded that a translocation of a segment of d that is greater the level of resolution of the map has occurred.

It is concluded that a translocation event linking a to d has occurred on one chromosome, and that a separate translocation linking d to g has occurred on a second chromosome. It is concluded that the sample is heterozygous for each translocation event.

### Example 5

### Conversion of Read Pair Separations into Kernels

Read pair data from human chromosome 7 (15 Mb) is obtained, the read pairs are organized into 200 bp bins, and an LLR value is calculated for each of the bins. A high LLR value is obtained which corresponded to a known heterozygous inversion (FIG. 5A). In the same analysis region, a 10 Kb heterozygous deletion kernel, and a 26 Kb heterozygous duplication (L) kernel was identified (FIG. 5B and FIG. 5C, respectively).

### Example 6

### Identification of a Displaced Segment

Read pair information is obtained for a sample, and the read pairs are mapped relative to a reference scaffold. A local density variation representing a potential misplaced segment of read pairs mapping to a segment of the scaffold is observed as a vertical or horizontal band of unexpectedly high read density (FIG. 16A). A corresponding horizontal or vertical band of unexpectedly low read density “hole” is identified, and the expected read pair density for this band is compared to that of the misplaced segment. The expected read pair density for the hole matches the observed density for the band, and it is concluded that the misplaced segment corresponds to the hole. The scaffold model is adjusted by swapping the misplaced segment with the hole to generate an improved model.

### Example 7

### Identification of a Collapsed Segment in a Diploid Genome

Read pair information is obtained for a sample, and the read pairs are mapped relative to a reference scaffold. For a section of the scaffold A-B-B′, a first area of higher than expected density is observed near the central axis for segment A, relative to at least one other area near the central axis. A second area of unexpectedly low read density, in some cases manifested as a square or rectangular shape of low density dividing two segments (FIG. 16A), is also observed with one corner of the second area contacting the central axis between B and B′. The “excess” density in the first area is approximately proportional to the combined density corresponding to the lack of observed density in the second area. It is concluded that the first area corresponds to a diploid sequence of A that has been collapsed due to high similarity, and the lack of density on or near the axis between B and B′ indicates an improper join has occurred. Optionally, the scaffold is adjusted by duplicating A (increasing the copy number) and breaking B-B′ to create two separate chromosomes comprising A-B or A-B′.

### Example 8

### Identification of a Collapsed Repeat and Rejoin in a Diploid Genome

Read pair information is obtained for a sample, and the read pairs are mapped relative to a reference scaffold. For a section of the scaffold A-B/X-Y, a first area of higher than expected density is observed near the central axis for segment B/X, relative to at least one other area near the central axis, for example segments A or Y. Additionally, a second area of unexpectedly low read density, in some cases manifested as a square or rectangular shape of low density dividing two segments (FIG. 16B), is also observed with one corner of the second area not fully contacting the central axis between A and Y. It is concluded that second area corresponding to B/X comprises a collapsed segment, and A and Y have been improperly joined through a common fragment B/X. Optionally, the scaffold is adjusted by duplicating B/X, and breaking B-Y to create two separate chromosomes comprising A-B or X-Y.

### Example 9

### Identification of a Chromosome Break

Read pair information is obtained for a sample, and the read pairs are mapped relative to a reference scaffold. For a section of the scaffold, a lower than expected read density on and off the central axis is observed for an area corresponding to the connection between two segments. It is concluded that a chromosomal break is present, and the scaffold is updated accordingly.

### Example 10

### Identification of a Haploid Collapsed Segment

Read pair information is obtained for a sample of a haploid genome, and the read pairs are mapped relative to a reference scaffold. For a section of the scaffold, a higher than expected read density on the central axis (e.g. higher than the mean read density at other areas on the scaffold, near the axis) is observed for an area corresponding to the connection between two segments. No other significant off-axis features are identified. It is concluded that the area of high density represents a repeat segment that has been collapsed during assembly of the scaffold. The repeat segment is duplicated and placed adjacent to the original segment in the scaffold. Optionally, the model is iteratively adjusted until the read density near the axis at the repeated segment approximates the average read density at positions along the scaffold, indicating the correct number of repeat segments are present in the scaffold model.

### Example 11

### Genome Modeling

Read pair information is obtained for a tumor sample, and the read pairs are mapped relative to a human genome reference scaffold. A significant number of discrepancies between the scaffold and the read pair data are observed, manifested by changes between expected and observed density for a plurality of areas, which complicates analysis. Each discrepancy is given a score based on the size of the discrepancy. The scaffold is remodeled as a collection of weighted genomes, each comprising weighted chromosomes, and the read pair data is remapped. This results in a significant decrease in the number of discrepancies, and hence the score. As a result, analysis of the data proceeds normally and information about the heterogeneity of the tumor cell population is obtained. Optionally, the model is adjusted iteratively to further lower the score and obtain a better fit for the read pair data to the scaffold, as exemplified in FIG. 17A.

### Example 12

### Graph Representation of a Scaffold

Read pair information is obtained for a sample, and the read pairs are mapped relative to a reference scaffold. Segments of the scaffold are represented mathematically as nodes, and areas of mapped read density are represented as edges connecting the nodes. Optionally, each edge is weighted as function of the likelihood that connection between segments is correct (e.g. blocking edges) based on the observed areas and locations of read density. Computational algorithms are employed to iteratively evaluate paths through the nodes, following the edges until the shortest path is identified. Optionally, a machine learning algorithm is employed to find the shortest paths through the graph. It is concluded that the shortest path represents the best fit scaffold model for the read pair data. Representing the assembly scaffold as a graph in this manner leads to an overall decrease in computational time and energy required to generate a best fit scaffold model.

### Example 13

### Diploid Inversion

A sample comprising diploid genome is suspected of having at least some genomic material having undergone at least one genomic rearrangement relative to a reference scaffold. The sample comprises a first chromosome having segments a, b, and c, and second chromosome comprising segments d, e, and f.

Read pair information is obtained for the sample, and the read pairs are mapped relative to the reference scaffold.

A local density variation representing a substantial underrepresentation of read pairs mapping to segments a-b and b-c is observed. It is concluded that a rearrangement bringing a and the right end of b along with the left end of b with c has occurred (inversion).

The local density variation is analyzed in further detail. It is observed that, at the peak density for this local density variation, read pair bin occupancy, as a measurement of density, is only half that of read pair density immediately off the axis. Furthermore, the displaced density is present as a “bowtie” pattern located off-axis, at the midpoint between segment b. It is concluded that the inversion has only taken place on one chromosome.

The local density variation is observed as to its symmetry. It is seen that the local density variation is substantially bilaterally symmetric along a line bisecting its right angle edge nearest to the scaffold axis with the level of resolution of the mapping. It is concluded that the event is a simple inversion reverse the orientation of segment b.

### Example 14

### Diagnostic Methods

A tumor sample is collected from a patient, sequenced to obtain read pair data, and the resulting data mapped onto a human reference genome scaffold. Off-axis “bowtie” density features are identified using the methods and systems herein, and these features are identified as a translocation between genes ETV6 and NTRK3 for one or both chromosomes to form a fusion, as shown in FIG. 7. The difference between expected density and observed density of the features indicates the percent of chromosomes in the genomes of tumor cells having the mutation. From this result, and optionally additional features present or absent from the read pair data, the patient is diagnosed with a cancer, such as a mammary analogue secretory carcinoma, and subsequently treated with a drug known to target cancers with this mutation, such as an NTRK3 kinase inhibitor. Sequencing of a sample removed from the tumor after completing a treatment regimen indicate a reduction or elimination of density for features corresponding to the ETV6-NTRK3 translocation. A clinician concludes that the drug treatment has successfully killed off tumor cells having the translocation in their genomes.

### Example 15

### Diagnostic Methods

A tumor sample is collected from a patient, sequenced to obtain read pair data, and the resulting data mapped onto a human reference genome scaffold. Off-axis “bowtie” density features, corresponding to a translocation between genes ETV6 and NTRK3 are not observed for one or both chromosomes using the methods and systems herein. From this result, and optionally additional features present or absent from the read pair data, a clinician concludes that the patient does not require treatment with an drug, such as an NTRK3 kinase inhibitor.

What is claimed is:

1. A method comprising:
mapping read pair sequence information onto a sequence scaffold; and
identifying a local variation in density of a plurality of read pair symbols so mapped.

2. The method of claim 1, comprising assigning the local variation in density to a corresponding structural arrangement feature.

3. The method of claim 1, comprising restructuring the sequence scaffold so that the local variation in density is reduced.

4. The method of claim 1, wherein mapping read pair sequence information onto a sequence scaffold comprises positioning a symbol indicative of a read pair such that distance of the symbol from an axis representative of the sequence scaffold indicates distance from a mapped position of a first read of a read pair on the sequence scaffold to a mapped position of a second read of the read pair on the sequence scaffold, and such that position of the symbol relative to the axis representative of the sequence scaffold indicates an average of the mapped position of the first read of the read pair and the mapped position of the second read of the read pair

5. The method of claim 2, wherein restructuring the sequence scaffold comprises reordering at least some contigs of the sequence scaffold.

6. The method of claim 2, wherein restructuring the sequence scaffold comprises reorienting at least one contig of the sequence scaffold.

7. The method of claim 2, wherein restructuring the sequence scaffold comprises introducing a break into at least one contig of the sequence scaffold.

8. The method of claim 7, further comprising introducing a sequence present at one edge of the break onto a second edge of the break.

9. The method of claim 1, wherein restructuring the sequence scaffold comprises translocating a segment of a first contig into an internal region of a second contig.

10. The method of claim 1, wherein mapping read pair sequence information onto a sequence scaffold comprises assigning read pair information to a plurality of bins.

11. The method of claim 1, wherein identifying a local variation in density comprises identifying a region having a locally low density of symbols.

12. The method of claim 1, wherein identifying a local variation in density comprises identifying a region having a locally high density of symbols.

13. The method of claim 1, wherein identifying a local variation in density comprises identifying a density at a first position and a density at a second position, wherein the density at the first position and the density at the second position differ significantly.

14. The method of claim 13, wherein the first position and the second position are adjacent.

15. The method of claim 13, wherein the first position and the second position are equidistant from the sequence scaffold.

16. The method of claim 1, wherein identifying a local variation in density comprises obtaining an expected density at a first position and an observed density at the first position.

17. The method of claim 16, wherein the expected density at the first position is a density predicted by density gradient that decreases monotonically with increased distance from the axis representative of the sequence scaffold.

18. The method of claim 1, wherein a local density variation of a fraction of a whole number value equal to a ploidy of a sample indicates an event in that proportion of a sample ploidy complement.

19. The method of claim 1, wherein the scaffold represents a cancer cell genome.

20. The method of claim 1, wherein the scaffold represents a transgenic cell genome.

21. The method of claim 1, wherein the scaffold represents a gene-edited genome.

22. The method of claim 3, wherein the scaffold has an N50 of at least 20% greater following the restructuring.

23. A method comprising
obtaining a scaffold comprising sequence scaffold information;
obtaining paired read information;
deploying the paired read information such that at least some read pair information is depicted so as to indicate position of each read in a read pair relative to the scaffold and to indicate distance of one read to another as mapped on the scaffold; and
identifying a local variation in density of the paired read information as deployed.

24. The method of claim 23, comprising assigning the local variation in density to a corresponding structural arrangement feature.

25. The method of claim 23, comprising reconfiguring the scaffold so as to decrease the local variation.

26. The method of claim 23, wherein obtaining a scaffold comprising sequence scaffold information comprises sequencing a nucleic acid sample.

27. The method of claim 23, wherein obtaining a scaffold comprising sequence scaffold information comprises receiving digital information representative of a nucleic acid sample.

28. The method of claim 23, comprising obtaining a predicted density distribution for deployed read pair information.

29. The method of claim 28, wherein the identifying comprises identifying a significant difference between the predicted density distribution and the depicted read pair information density.

30. The method of claim 23, wherein identifying a local variation comprises identifying a density perturbation having a density peak at an apex of a right angle.

31. The method of claim 30, wherein the apex of the right angle points to an axis representative of the scaffold.

32. The method of claim 23, wherein obtaining paired end read information comprises crosslinking unextracted nucleic acids.

33. The method of claim 23, wherein obtaining paired end read information comprises crosslinking nucleic acids bound in chromatin.

34. The method of claim 33, wherein the chromatin is native chromatin.

35. The method of claim 23, wherein obtaining paired end read information comprises binding a nucleic acid to a nucleic acid binding moiety.

36. The method of claim 23, wherein obtaining paired end read information comprises generating reconstituted chromatin.

37. The method of claim 23, wherein deploying the paired read information comprises assigning read pair information to a plurality of bins.

38. The method of claim 23, wherein restructuring the sequence scaffold comprises reordering at least some contigs of the sequence scaffold.

39. The method of claim 25, wherein restructuring the sequence scaffold comprises reorienting at least one contig of the sequence scaffold.

40. The method of claim 25, wherein restructuring the sequence scaffold comprises introducing a break into at least one contig of the sequence scaffold.

41. The method of claim 40, further comprising introducing a sequence at one edge of the break onto a second edge of the break.

42. The method of claim 25, wherein restructuring the sequence scaffold comprises translocating a segment of a first contig into an internal region of a second contig.

43. The method of claim 23, wherein the scaffold represents a cancer cell genome.

44. The method of claim 23, wherein the scaffold represents a transgenic cell genome.

45. The method of claim 23, wherein the scaffold represents a gene-edited genome.

46. The method of claim 23, wherein the scaffold has an N50 of at least 20% greater following the restructuring.

47. The method of claim 23, wherein a local density variation of a fraction of a whole number value equal to a ploidy of a sample indicates an event in that proportion of a sample ploidy complement.

48. A method of identifying a structural rearrangement in a sample relative to a sequence scaffold, comprising
mapping read pair sequence information onto a sequence scaffold;
identifying local density variation having a right angle edge pointing to an axis corresponding to the sequence scaffold and having bilateral symmetry along a line that bisects the right angle edge; and
categorizing the sample as having a simple translocation relative to the sequence scaffold comprising segments of lengths from a translocation point at least as long as the longest furthest mapped read of the local density variation.

49. A method of identifying a structural rearrangement in a sample, comprising
mapping read pair sequence information onto a sequence scaffold;
identifying local density variation having a right angle edge pointing to an axis corresponding to the sequence scaffold;
identifying a sub-region of the local density variation that disrupts bilateral symmetry along a line that bisects the right angle edge; and
categorizing the sample as having a translocation relative to the sequence scaffold comprising a segment that lacks sequence to which a population of symmetry-restoring read pairs would map.

50. A method of identifying a structural rearrangement in a sample relative to a sequence scaffold, comprising
mapping read pair sequence information onto a sequence scaffold;
identifying local density variation having a right angle edge pointing to an axis corresponding to the sequence scaffold;
obtaining an expected read pair density distribution curve; and
identifying scaffold segments to which read pairs comprising the local density variation map;
repositioning the scaffold segments such that the read pairs comprising the local density variation map to a region indicated by the expected read pair density distribution curve to have a density of the local density variation.

51. A computer monitor configured to display results of the method of any one of claims 1-50.

52. A computer system configured to perform computational steps of the method of any one of claims 1-50.

53. A visual representation of mapped read pair data of any one of claims 1-50.
